(19) United States
(12) Patent Application Publication
(10) Pub. No.: US 2013/0244929 A1
(43) Pub. Date: Sep. 19, 2013

(54) BRIDGED LIPOGLYCOPEPTIDES THAT POTENTIATE THE ACTIVITY OF BETA-LACTAM ANTIBACTERIALS

(71) Applicants: Michel Gallant, Pierrefonds (CA); Karine Villeneuve, Montreal (CA); Patrick Beaulieu, Laval (CA); Joel Robichaud, Dollard des Ormeaux (CA); Helene Juteau, St-Laurent (CA); Yves Gareau, Ile Perrot (CA); Sherman T. Waddell, Westfield, NJ (US); Nancy Kevin, East Brunswick, NJ (US); Xin Gu, Scotch Plains, NJ (US); Joann Huber, Westfield, NJ (US); Michael J. Salvatore, JR., Iselin, NJ (US); Kenneth Wilson, Westfield, NJ (US); Scott K. Smith, Harpswell, ME (US); Deborah Zink, Manalapan, NJ (US)

(72) Inventors: Michel Gallant, Pierrefonds (CA); Karine Villeneuve, Montreal (CA); Patrick Beaulieu, Laval (CA); Joel Robichaud, Dollard des Ormeaux (CA); Helene Juteau, St-Laurent (CA); Yves Gareau, Ile Perrot (CA); Sherman T. Waddell, Westfield, NJ (US); Nancy Kevin, East Brunswick, NJ (US); Xin Gu, Scotch Plains, NJ (US); Joann Huber, Westfield, NJ (US); Michael J. Salvatore, JR., Iselin, NJ (US); Kenneth Wilson, Westfield, NJ (US); Scott K. Smith, Harpswell, ME (US); Deborah Zink, Manalapan, NJ (US)

(21) Appl. No.: 13/583,462

(22) PCT Filed: Mar. 4, 2011
(86) PCT No.: PCT/US11/27159

§ 371 Date: May 22, 2013

Related U.S. Application Data
(60) Provisional application No. 61/312,058, filed on Mar. 9, 2010.

(51) Int. Cl. A61K 38/08; A61K 31/407; C07K 7/02

(52) U.S. Cl. CPC A61K 38/08; C07K 7/02; A61K 31/407
USPC 514/2.7; 530/323; 514/2.9

(57) ABSTRACT

The present invention provides novel lipoglycopeptide compounds which are Type 1 signal peptidase inhibitors (SpsB). Compounds of the present invention are useful for the treatment of various bacterial related infectious diseases, particularly when used as a potentiator of a β-lactam antibiotic such as imipenem and ertapenem. Accordingly, the present invention provides a method for the treatment of bacterial related infections using the compounds described herein, either alone or in combination with a β-lactam antibiotic.

### CROSS-REFERENCE TO RELATED APPLICATIONS

Not Applicable

### FIELD OF THE INVENTION

The present invention relates to novel bridged lipoglycopeptides which are inhibitors of bacterial signal peptidase. The compounds of the invention are useful as antibacterial agents alone, and as potentiators in combination with β-lactam antibiotics, for the treatment of bacterial infections, particularly those involving drug-resistant Staphylococcus sp. Accordingly, this invention also relates to methods of treating bacterial infections in mammals (e.g., humans) which comprises administering, optionally together with a β-lactam antibiotic, a therapeutically effective amount of a compound of formula (I) including pharmaceutically acceptable salts, prodrugs, anhydrides, and solvates thereof.

### BACKGROUND OF THE INVENTION

β-lactam antibiotics interfere with the assembly of peptidoglycan in the bacterial cell wall by inhibiting enzymatic reactions involved in the final stages of assembly. β-lactams antibiotics are among the most widely used antibiotics due to their relatively high effectiveness and low side effects. See Wilke et al., 2005, Curr Opin Microbial 8:525-533. However, drug resistance is a major problem with β-lactam antibiotics. For example, MRSA is a major cause of nosocomial and community-acquired illnesses throughout the world and accounts for ˜60% of all staphylococcal infections. Infection with MRSA results in diverse clinical manifestations ranging from minor skin and soft tissue infections to life threatening endocarditis, bacteremia and pneumonia. Due to the prevalence of resistance mechanisms in MRSA and other bacteria, new ways of overcoming this resistance, especially through unique combinations of antibiotic targets, are desirable.

Citation or identification of any reference in this section or any other section of this application shall not be construed as an indication that such reference is available as prior art to the present invention.

### SUMMARY OF THE INVENTION

The present invention provides novel bridged lipoglycopeptide compounds which are type I bacterial signal peptidase inhibitors (SpsB). The compounds, or pharmaceutically acceptable salts thereof, are useful alone, or in combination with β-lactam antibiotics, for the treatment of bacterial infections. In particular, the present invention relates to compounds of Formula I, and pharmaceutically acceptable salts thereof:

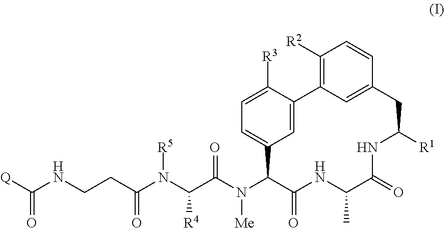

wherein

R¹ is selected from C(R⁶)O, C(R⁶)₂OR⁶, COOR⁶ or CONR⁷R⁸;

R² and R³ are independently selected from H, halogen, OR⁶, SR⁶, SO₂R⁶, and NR⁷R⁸;

R⁴ and R⁵ are independently selected from hydrogen, C₁ to C₂₁ alkyl, cycloalkyl, alkenyl, cycloalkenyl and aryl, wherein the alkyl, cycloalkyl, alkenyl, cycloalkenyl or aryl is optionally substituted with one or more of C₁ to C₄ alkyl, —NR⁷R⁸, guanidine, —OR⁶, OCONR⁷R⁸, COR⁶, CONR⁷R⁸, CN, SOR⁶, SO₂R⁶, SO₂NR⁷R⁸, F, Cl, Br, I or CF₃;

or R⁴ and R⁵ together with the atoms to which they are directly attached form a O-5 to 5-membered heterocycle, optionally substituted with one or more of C₁ to C₄ alkyl, —NR⁷R⁸, guanidine, —OR⁶, OCONR⁷R⁸, COR⁶, CONR⁷R⁸, CN, SOR⁶, SO₂R⁶, SO₂NR⁷R⁸, F, Cl, Br, I or CF₃;

Q is AryA or HetA;

AryA is an aryl optionally substituted with one or more of AryB, R⁶ optionally substituted with AryB, C₁ to C₂₁ alkyl optionally substituted with AryB, C₁ to C₂₁ alkenyl optionally substituted with AryB, or C₁ to C₂₁ alkynyl optionally substituted with AryB;

HetA is a heteroaryl optionally substituted with one or more of AryB, R⁶ optionally substituted with AryB, C₁ to C₂₁ alkyl optionally substituted with AryB, C₁ to C₂₁ alkenyl optionally substituted with AryB, or C₁ to C₂₁ alkynyl optionally substituted with AryB;

AryB is an aryl optionally substituted with a C₁ to C₂₁ alkyl or phenyl;

R⁶, R⁷, R⁸, are independently selected from H and C₁ to C₆ alkyl, wherein the alkyl is optionally substituted with one or more of —OR⁹, OCONR¹⁰R¹¹, OCOR⁹, COR⁹, CO₂R⁹, CONR¹⁰R¹¹, CN, SOR⁹, SO₂R⁹, SO₂NR¹⁰R¹¹, F, Cl, Br, I or CF₃ and

R⁹, R¹⁰, and R¹¹ are independently selected from H and C₁ to C₄ alkyl.

In a first embodiment, R¹ is CH₂OH, COOH or CONH₂ and the other substituents are as provided in the general formula for compound T.

In a second embodiment, R² and R³ are independently selected from H and OR⁶ and the other substituents are as provided in the first embodiment or the general formula for compound I.

In a third embodiment, R² and R³ are independently selected from H, OH, and OCH₃ and the other substituents are as provided in the first embodiment or the general formula for compound I.

In a fourth embodiment, R⁴ and R⁵ are H or C₁ to C₂₁ alkyl, wherein the alkyl is optionally substituted with amine, guanidine or —NR⁷R⁸ and the other substituents are as provided in any of the first, second, or third embodiments or the general formula for compound I.

In a fifth embodiment, R⁷ and R⁸ are independently selected from C₁ to C₆ alkyl and the other substituents are as provided in any of the first, second, third, or fourth embodiments or the general formula for compound I.

In a sixth embodiment, Q is

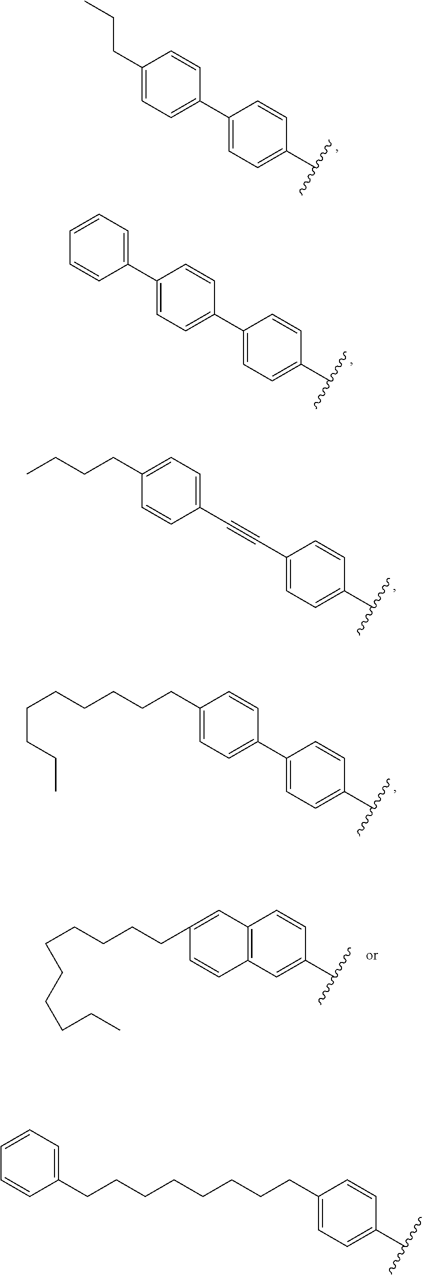 [SMILES: *c1ccc(-c2ccc(CCC)cc2)cc1 ; *c1ccc(-c2ccc(-c3ccccc3)cc2)cc1 ; *c1ccc(C#Cc2ccc(CCCC)cc2)cc1 ; *c1ccc(-c2ccc(CCCCCCCCC)cc2)cc1 ; *c1ccc2cc(CCCCCCCCCC)ccc2c1 ; *c1ccc(CCCCCCCCc2ccccc2)cc1]

and the other substituents are as provided in any of the first, second, third, fourth, or fifth embodiments or the general formula for compound I.

In a seventh embodiments, Q is

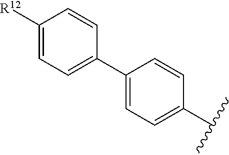

wherein R¹² is a C₁ to C₁₂ alkyl and the other substituents are as provided in any of the first, second, third, fourth, or fifth embodiments or the general formula for compound I.

In another embodiment of the invention, the compound of the invention is selected from the exemplary species depicted in Examples 1 through 20 shown below (as the free base or a pharmaceutically acceptable salt thereof).

In one aspect, the invention provides a method for treating a bacterial infection in a patient, preferably a human, where the treatment includes administering a therapeutically or pharmacologically effective amount of a compound of formula I, or a pharmaceutically acceptable salt thereof, and a pharmaceutically acceptable carrier. In another aspect, the invention provides a method for treating a bacterial infection in a patient, preferably a human, where the treatment includes administering a therapeutically or pharmacologically effective amount of a combination of 1) a β-lactam antibiotic; and 2) a compound of formula I, or a pharmaceutically acceptable salt thereof; and 3) a pharmaceutically acceptable carrier.

In embodiments where a β-lactam antibiotic is used in combination with a compound of formula I, the β-lactam antibiotic may be a carbapenern, cephalosporin, monobactam or penicillin. Exemplary carbapenem antibiotics useful in the methods of the invention include ertapenem, imipenem and meropenem. In some embodiments of the invention, the β-lactam may be administered with a β-lactamase inhibitor. In some embodiments of the invention, the carbapenem may be administered with a DHP inhibitor, e.g., cilastatin.

In the various embodiments of the invention where a compound of formula I and a β-lactam antibiotic are used in combination, the β-lactam antibiotic and compound of formula I can be administered sequentially or concurrently. Preferably, the β-lactam antibiotic and compound of formula I are administered together. When administered concurrently, the β-lactam antibiotic and compound of formula I may be administered in the same formulation or in separate formulations. When administered sequentially, either the β-lactam or compound of formula I may be administered first. After administration of the first compound, the other compound is administered, for example, within from 1 to 60 minutes, e.g., within 1, 2, 3, 4, 5, 10, 15, 30, or 60 minutes. In one aspect of the invention, when a β-lactamase inhibitor is used, it may be administered separately, or in a formulation with the compound of formula I and/or β-lactam antibiotic. In one aspect of the invention, when a DHP inhibitor is used to improve the stability of a carbapenem, it may be administered separately, or in a formulation with the compound of formula I and/or carbapenem.

The invention further provides pharmaceutical compositions comprising a compound of formula I, a pharmaceutically acceptable carrier, and optionally a β-lactam antibiotic. In embodiments where a combination is used, the β-lactam antibiotic and the compound of formula I, are present in such amounts that their combination constitutes a therapeutically effective amount. Due to the potentiating effects of the compound of formula I, the amount of β-lactam antibiotic present in a combination may be less that of a β-lactam antibiotic used alone. In certain embodiments, the composition further comprises a β-lactamase antibiotic.

In embodiments where the β-lactam antibiotic is a carbapenem, the invention further provides pharmaceutical compositions comprising a carbapenem antibiotic, a DHP inhibitor, a compound of formula I, and a pharmaceutically acceptable carrier. In embodiments where the β-lactam antibiotic is a carbepenem, the carbapenem antibiotic is preferably selected from the group consisting of ertapenem, imipenem and meropenem.

The invention also includes a compound of the present invention for use (i) in, (ii) as a medicament for, or (iii) in the preparation of a medicament for treating a bacterial infection. In these uses, the compounds of the invention can optionally be employed in combination with one or more additional therapeutical agents including a β-lactam antibiotic.

### BRIEF DESCRIPTION OF THE DRAWINGS

FIG. 1: An SpsB inhibitor and Imipenem synergize to inhibit bacterial growth in vitro. The MIC of SpsB inhibitor on the MRSA strain COL was assessed by varying the concentration of the compound (0.016-16 μg/ml) in the absence and presence of various concentrations of Imipenem (0.5-32 μg/ml) and FIC values were generated for both compounds.

FIGS. 2A-B: An SpsB inhibitor and Imipenem synergize to inhibit bacterial growth in disseminated (A) and deep thigh (B) MRSA infection mouse models.

### DETAILED DESCRIPTION OF THE INVENTION

The present invention is based in part on Applicants' discovery of compounds which have antibacterial activity and inhibit bacterial type I signal peptidase activity. Compounds of formula I are useful in the treatment of various bacterial related infections alone or in combination with β-lactam antibiotics to potentiate the in vivo effects of β-lactam antibiotics, particularly in methicillin-resistant strains of Staphylococcus aureus and Staphylococcus epidermis.

The present invention also relates to the use of a compound of formula I, or a pharmaceutically acceptable salt thereof, in the treatment of bacterial infections. In one embodiment, a compound of formula I is used in combination with a β-lactam antibiotic to potentiate the in vivo effects of the β-lactam antibiotic for the treatment of bacterial infections, i.e., as a potentiator. Compounds of the present invention, in combination with other antimicrobials, such as imipenem and ertapenem, preferably act synergistically, i.e., as potentiators. This combination is particularly useful against infections arising from bacteria which are resistant to one or more antibacterial agents, e.g., methicillin-resistant S. aureus, methicillin-resistant coagulase-negative Staphylococcus and methicillin-resistant S. epidermis.

SpsB, a staphylococcal Type I signal peptidase, is a membrane-localized serine protease responsible for cleaving the N-terminal signal peptides from most secreted precursor proteins during export and maturation. While there are three types of bacterial signal peptidases, only type I signal peptidases in bacteria have been found to be essential for viability. See Paetzel et al., 2002, Chem Rev 102:4549-4579. Bacterial SPase I and eukaryotic signal peptidase are thought to have distinctive catalytic mechanisms, potentially limiting the cross-reactivity of an agent specifically targeting bacterial SPase I. See Sung et al., 1992, J. Biol. Chem. 267:13154-13159; Black, M. T., 1993, J. Bacterial. 175:4957-4961; Tschantz et al., 1993, J. Biol. Chem. 268:27349-27354. Furthermore, the location of active domain of bacterial SPase I on the cytoplasmic membrane, as opposed to the location of eukaryotic signal peptidase in the lumenal side of the microsomes, makes bacterial SPase I a particularly attractive target. See id. Thus an inhibitor can access the staphylococcal target without cellular penetration. Without being bound by any theory, SpsB inhibitors could weaken the cell wall by preventing the localization of secreted enzymes required for cell wall biogenesis. Inhibitors could also cause accumulation of unprocessed proteins in the membrane, which could impede the many membrane localized reactions required for cell wall synthesis.

Staphylococcus aureus has two type I SPases; an active form responsible for the type I bacterial signal peptidase activity (SpsB) and an inactive form missing the catalytic residues (SpsA). See Paetzel et al., 2002, Chem Rev 102:4549-4579; Paetzel et al., 2000, Pharmacol & Ther 87:27-49. Certain β-lactam antibiotics, in particular, 5S stereoisomers, have been found to be inhibitors of SPase. Additional inhibitors of spsB have been described in Bruton et al., 2003, Eur J Med Chem 38:351-356; Kulanthaivel et al., 2004, J Biol Chem 279:36250-36258; Roberts et al., 2007, J Am Chem Soc 129:15830-15838; and U.S. Pat. No. 6,951,840.

In one aspect of the invention, a compound of formula I can enhance the activity of a β-lactam antibacterial agent by inducing susceptibility to the antibacterial agent in a drug-resistant strain such as MRSA. In another aspect of the invention, a compound of formula I can enhance the activity of a β-lactam antibacterial agent by reducing the dosage of the antibacterial agent need for a therapeutic effect in a drug-sensitive strain. For example, if a compound of formula I reduces the Minimum Inhibitory Concentration (MIC) of an antibacterial agent (where the MIC is the minimum concentration of antibacterial agent which will completely inhibit growth) in a susceptible strain, then such treatment may be advantageous to enable a reduction in the amount of antibacterial agent administered (could reduce side effects of an antibiotic), or to decrease the frequency of administration. In another aspect of the invention, compounds of formula I can enhance the activity of an antibacterial agent such as a carbapenem to prevent the emergence of a resistant sub-population in a heterogeneous bacterial population with a resistant sub-population.

Treatments using compounds of formula I as a potentiator represent a new approach to antibacterial therapy in which a compound of formula I can be administered together with a β-lactam antibiotic (either concurrently or sequentially) to allow effective treatment of an infection involving a resistant bacterium, or to reduce the amount of antibacterial agent necessary to treat an infection. Potentiators can be used to enhance the activity of antibacterial agents whose clinical efficacy has been limited by the increasing prevalence of resistant strains.

The compounds of the present invention are useful per se and in their pharmaceutically acceptable salt and ester forms as potentiators for the treatment of bacterial infections in animal and human subjects. The term “pharmaceutically acceptable ester, salt or hydrate”, refers to those salts, esters and hydrated forms of the compounds of the present invention which would be apparent to the pharmaceutical chemist, e.g., those which are substantially non-toxic and which may favorably affect the pharmacokinetic properties of said compounds, such as palatability, absorption, distribution, metabolism and excretion. Other factors, more practical in nature, which are also important in the selection, are cost of the raw materials, ease of crystallization, yield, stability, solubility, hygroscopicity and flowability of the resulting bulk drug. Conveniently, pharmaceutical compositions may be prepared from the active ingredients in combination with pharmaceutically acceptable carriers.

As used herein, the term “alkenyl” refers to a straight or branched-chain acyclic unsaturated hydrocarbon having a number of carbon atoms in the specified range and containing at least one double bond. Thus, for example, “C₂-C₃ alkenyl” refers to vinyl, (1Z)-1-propenyl, (1E)-1-propenyl, 2-propenyl, or isopropenyl.

As used herein, the term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl. Preferred alkyl groups include methyl, ethyl, propyl, isopropyl, butyl and hexyl.

As used herein, the term, “alkynyl”, refers to a straight or branched-chain acyclic unsaturated hydrocarbon having a number of carbon atoms in the specified range and containing at least one triple bond.

As used herein, the term “aryl” refers to a group thus contains at least one ring having at least 6 atoms, with up to three such rings being present, containing up to 14 atoms therein, with alternating (resonating) double bonds between adjacent carbon atoms. Examples include, but are not limited to, phenyl, biphenyl and the like, as well as rings which are fused, e.g., naphthyl, phenanthrenyl, fluorenonyl and the like. The preferred aryl groups are phenyl, naphthyl, and biphenyl. Aryl groups may likewise be substituted as defined. Preferred substituted aryls include phenyl, biphenyl and naphthyl.

As used herein, the term “heteroaryl” generally refers to a heterocycle as defined below in which the entire ring system (whether mono- or poly-cyclic) is an aromatic ring system. It may refer to a 5- or 6-membered monocyclic aromatic ring which consists of carbon atoms and one or more heteroatoms selected from N, O and S. Representative examples of heteroaryls include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl.

As used herein, the term “heterocycle” (and variations thereof such as “heterocyclic” or “heterocyclyl”) broadly refers to (i) a 4- to 8-membered, saturated or unsaturated monocyclic ring, (ii) a 7- to 12-membered bicyclic ring system, or (iii) an 11 to 16-membered tricyclic ring system; wherein each ring in (ii) or (iii) is independent of or fused to the other ring or rings and each ring is saturated or unsaturated, and the monocyclic ring, bicyclic ring system, or tricyclic ring system contains one or more heteroatoms (e.g., from 1 to 6 heteroatoms, or from 1 to 4 heteroatoms) independently selected from N, O and S and a balance of carbon atoms (the monocylic ring typically contains at least one carbon atom and the ring systems typically contain at least two carbon atoms); and wherein any one or more of the nitrogen and sulfur heteroatoms is optionally be oxidized, and any one or more of the nitrogen heteroatoms is optionally quaternized. The heterocyclic ring may be attached at any heteroatom or carbon atom, provided that attachment results in the creation of a stable structure. When the heterocyclic ring has substituents, it is understood that the substituents may be attached to any atom in the ring, whether a heteroatom or a carbon atom, provided that a stable chemical structure results.

Examples of a salt include alkali metal salts such as a sodium salt, a potassium salt and a lithium salt; alkaline earth metal salts such as a calcium salt and a magnesium salt; metal salts such as an aluminium salt, an iron salt, a zinc salt, a copper salt, a nickel salt and a cobalt salt; amine salts such as inorganic salts such as an ammonium salt and organic salts such as a benzylamine salt, a chloroprocaine salt, a dibenzylamine salt, a dibenzylethylenediamine salt, a dicyclohexylamine salt, a diethanolamine salt, a diethylamine salt, an ethylenediamine salt, a glucosamine salt, a guanidine salt, a morpholine salt, an N-benzyl-phenethylamine salt, an N-methylglucamine salt, an N,N-dibenzylethylenediamine salt, a phenylglycine alkyl ester salt, a piperazine salt, a piperidine salt, a procaine salt, a pyrrolidine salt, a t-octylamine salt, a tetramethylammonium salt, a triethylamine salt, and a tris(hydroxymethyl)aminomethane salt; and amino acid salts such as a glycine salt, a lysine salt, an arginine salt, an ornithine salt, a glutamate or an aspartate.

Pharmaceutically acceptable salts also include acid addition salts. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydrofluoride, hydroiodide, 2-hydroxyethanesulfonate, lactate, maleate, methanesulfonate, 2-naphthalenesulfonate, nicotinate, nitrate, oxalate, p-toluenesulfonate, pamoate, pectinate, perchlorate, persulfate, 3-phenylpropionate, phosphate, picrate, pivalate, propionate, succinate, sulfate tartrate, thiocyanate, tosylate, trifluoromethanesulfonate and undecanoate.

Pharmaceutically acceptable salts can be synthesized from the compounds disclosed herein by conventional chemical methods. Generally, the salts are prepared by reacting the free acid with stoichiometric amounts or with an excess of the desired salt-forming inorganic or organic base in a suitable solvent or various combinations of solvents. Alternatively, salts can be prepared from the corresponding sodium or potassium salt of the compounds disclosed herein using conventional ion exchange processes with cation exchange resins carrying the desired salt base.

Pharmaceutically acceptable esters are such as would be readily apparent to a medicinal chemist, and include, for example, those described in detail in U.S. Pat. No. 4,309,438 (thienamycin). Included within such pharmaceutically acceptable esters are those which are hydrolyzed under physiological conditions, e.g., biolabile esters.

Biolabile esters may be suitable for oral administration, due to good absorption through the stomach or intenstinal mucosa, resistance to gastric acid degradation and other factors. Examples of biolabile esters include compounds of the form COOM in which M represents an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, methoxymethyl, 1-cyclohexyloxycarbonyloxyethyl, phthalidyl and (2-oxo-5-methyl-1,3-dioxolen-4-yl)methyl. Additional examples of biolabile esters include compounds of the form COOM where M is indanyl and others described in detail in U.S. Pat. No. 4,479,947.

Pharmaceutically acceptable hydrate is used in the conventional sense to include the compounds of formula I in physical association with water.

As used herein, a “potentiator” or “potentiating compound” refers to a compound which has a synergistic effect on antibacterial activity when used with an antibacterial agent. Thus, a potentiator enhances the antibacterial effect of an antibacterial agent when the two compounds are used in combination. A potentiator does not have to, but may, have significant antibacterial activity when used alone at concentrations similar to its concentration in the combination use.

As used herein, “pro-drug” refers to compounds with a removable group attached to one or both of the carboxyl groups of compounds of formula I (e.g., biolabile esters). Groups which are useful in forming pro-drugs are apparent to the medicinal chemist from the teachings herein. Any of the compounds disclosed herein may also be used in any known prodrug form.

As used herein, “synergy” or “synergistic” refers to the effects of a combination of antibacterial agents wherein the antibacterial activity of the combination is greater than the sum of the activity of the individual antibactrial agents, particular in strains such as methicillin-resistant Staphylococcus aureus (MRSA), methicillin-resistant Staphyloccus epidermidis (MRSE), and other methicillin-resistant coagulase negative staphylococci (MRCNS). In one embodiment, synergy is defined as an FIC index of 0.5.

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, and so forth.

Unless otherwise indicated, all isomeric forms of these compounds, including racemic, enantiomeric and diastereomeric forms, whether isolated or in mixtures, are within the scope of the present invention. Also included within the scope of the present invention are tautomeric forms of the present compounds as depicted.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

When any variable occurs more than one time in any constituent or in any formula depicting and describing compounds described herein, its definition on each occurrence is independent of its definition at every other occurrence. Also, combinations of substituents and/or variables are permissible only if such combinations result in stable compounds.

### Bridged Lipoglycopeptides

The compounds of the present invention are bridged lipoglycopeptides having a biphenyl cyclic core. Various compounds having a biphenyl cyclic core have been disclosed, e.g., in Roberts et al., 2007, J Am Chem Soc 129:15830-15838 and U.S. Pat. No. 6,951,840. The compounds of the present invention differ from prior compounds by the presence of a unique lipopeptidic side chain attached to the cyclic core. In particular, the present invention provides a compound of formula I:

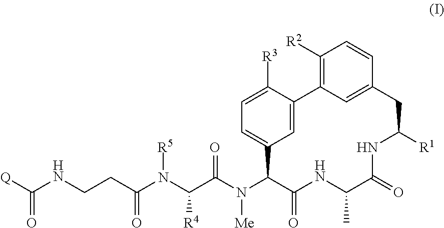

or a pharmaceutically acceptable salt wherein

R¹ is selected from C(R⁶)O, C(R⁶)₂OR⁶, COOR⁶ or CONR⁷R⁸;

R² and R³ are independently selected from H, halogen, OR⁶, SR⁶, SO₂R⁶, and

NR⁷R⁸;

R⁴ and R⁵ are independently selected from hydrogen, C₁ to C₂₁ alkyl, cycloalkyl, alkenyl, cycloalkenyl and aryl, wherein the alkyl, cycloalkyl, alkenyl, cycloalkenyl or aryl is optionally substituted with one or more of C₁ to C₄ alkyl, —NR⁷R⁸, guanidine, —OR⁶, OCONR⁷R⁸, COR⁶, CONR⁷R⁸, CN, SOR⁶, SO₂R⁶, SO₂NR⁷R⁸, F, Cl, Br, I or CF₃;

or R⁴ and R⁵ together with the atoms to which they are directly attached form a 4- to 5-membered heterocycle, optionally substituted with one or more of C₁ to C₄ alkyl, —NR⁷R⁸, guanidine, —OR⁶, OCONR⁷R⁸, COR⁶, CONR⁷R⁸, CN, SOR⁶, SO₂R⁶, SO₂NR⁷R⁸, F, Cl, Br, I or CF₃;

Q is AryA or HetA;

AryA is an aryl optionally substituted with one or more of AryB, R⁶ optionally substituted with AryB, C₁ to C₂₁ alkyl optionally substituted with AryB, C₁ to C₂₁ alkenyl optionally substituted with AryB, or C₁ to C₂₁ alkynyl optionally substituted with AryB;

HetA is a heteroaryl optionally substituted with one or more of AryB, R⁶ optionally substituted with AryB, C₁ to C₂₁ alkyl optionally substituted with AryB, C₁ to C₂₁ alkenyl optionally substituted with AryB, or C₁ to C₂₁ alkynyl optionally substituted with AryB;

AryB is an aryl optionally substituted with a C₁ to C₂₁ alkyl or phenyl;

R⁶, R⁷, R⁸, are independently selected from H and C₁ to C₆ alkyl, wherein the alkyl is optionally substituted with one or more of —OR⁹, OCONR¹⁰R¹¹, OCOR⁹, COR⁹, CO₂R⁹, CONR¹⁰R¹¹, CN, SOR⁹, SO₂R⁹, SO₂NR¹⁰R¹¹, F, Cl, Br, I or CF₃ and

R⁹, R¹⁰, and R¹¹ are independently selected from H and C₁ to C₄ alkyl.

In a first embodiment, R¹ is CH₂OH, COOH or CONH₂ and the other substituents are as provided in the general formula for compound I.

In a second embodiment, R² and R³ are independently selected from H and OR⁶ and the other substituents are as provided in the first embodiment or the general formula for compound I.

In a third embodiment, R² and R³ are independently selected from H, OH, and OCH₃ and the other substituents are as provided in the first embodiment or the general formula for compound I.

In a fourth embodiment, R⁴ and R⁵ are H or C₁ to C₂₁ alkyl, wherein the alkyl is optionally substituted with amine, guanidine or —NR⁷R⁸ and the other substituents are as provided in any of the first, second, or third embodiments or the general formula for compound I.

In a fifth embodiment, R⁷ and R⁸ are independently selected from C₁ to C₆ alkyl and the other substituents are as provided in any of the first, second, third, or fourth embodiments or the general formula for compound I.

In a sixth embodiment, Q is

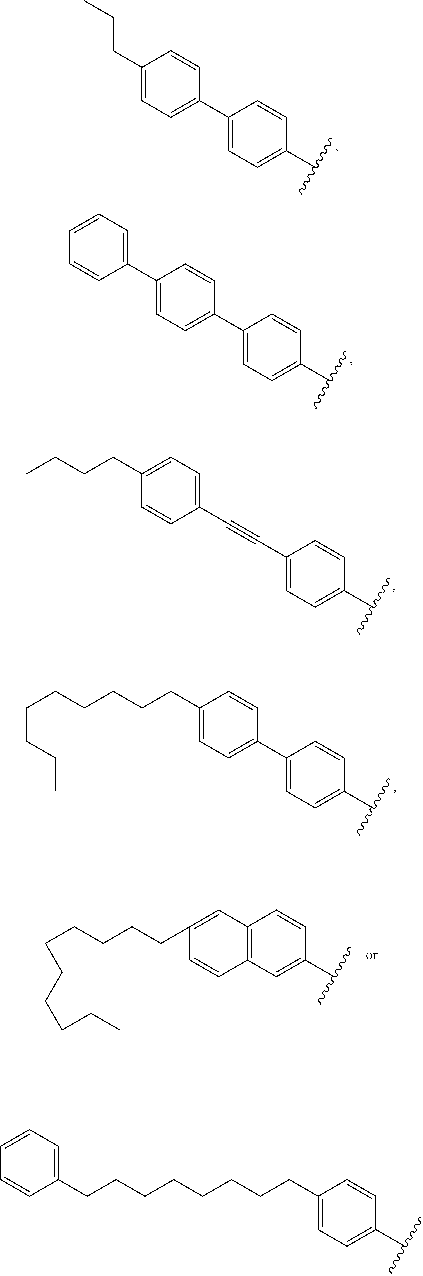 [SMILES: *c1ccc(-c2ccc(CCC)cc2)cc1 ; *c1ccc(-c2ccc(-c3ccccc3)cc2)cc1 ; *c1ccc(C#Cc2ccc(CCCC)cc2)cc1 ; *c1ccc(-c2ccc(CCCCCCCCC)cc2)cc1 ; *c1ccc2cc(CCCCCCCCCC)ccc2c1 ; *c1ccc(CCCCCCCCc2ccccc2)cc1]

and the other substituents are as provided in any of the first, second, third, fourth, or fifth embodiments or the general formula for compound I.

In a seventh embodiments, Q is

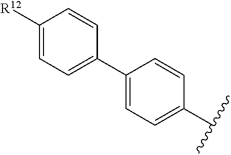

wherein R¹² is a C₁ to C₁₂ alkyl and the other substituents are as provided in any of the first, second, third, fourth, or fifth embodiments or the general formula for compound I.

In any of the above embodiments, where the substitution is a C₁ to C₂₁ alkyl, C₁ to C₂₁ alkenyl, or C₁ to C₂₁ alkynyl, a C₁ to C₁₂, C₁ to C₁₀, or C₁ to C₉ group may be used instead.

In the embodiments of the compounds as provided above, it is to be understood that each embodiment may be combined with one or more other embodiments, to the extent that such a combination provides a stable compound and is consistent with the description of the embodiments. It is further understood that the embodiments of the compositions and methods provided herein are understood to include all embodiments of the compounds, including such embodiments that result from combinations of embodiments of the compound.

In addition, it is understood that, in the description of embodiments of the compounds as set forth above, indicated substitutions are included only to the extent that the substituents provide stable compounds consistent with the definition.

In certain embodiments of the invention, the compound of formula I is one of the following, including a pharmaceutically acceptable salt thereof:
6-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
5-(R,S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
(5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
(5R)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
(5R,S)-6-[[(7S,10S,13S)-3,18-dihydroxy-13-(hydroxymethyl)-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(T-propylbiphenyl-4-yl)carbonyl]amino}propanoyl)amino]hexan-1-aminium;
6-[[(7S,10S,13S)-13-(aminocarboxyl)-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-({3-[(1,1′:4′,1″-terphenyl-4-ylcarbonyl)amino]propanoyl}amino)hexan-1-aminium;
({(4S)-5-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-oxo-4-[(3-{[(4′-propylbiphenyl-4-yl)carbonyl]amino}propanoyl)amino]pentyl}amino)(imino)methanaminium;
6-[[(7S,10S,13S)-13-carboxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
5-{[3-({4-[(4-butylphenyl)ethynyl]benzoyl}amino)propanoyl]amino}-6-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxohexan-1-aminium;
4-[{2-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-2-oxoethyl}(3-{[(4′-propylbiphenyl-4-yl)carbonyl]amino}propanoyl)amino]butan-1-aminium;
(8S,11S,14S)-3,18-dihydroxy-11-methyl-14-(methyl{[(2S)-1-(3-{[(4′-propylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(8S,11S,14S)-3,18-dimethoxy-11-methyl-14-(methyl{[(2R)-1-(3-{[(4′-propylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(8S,11S,14S)-3,18-dihydroxy-11-methyl-14-(methyl{[(2R)-1-{3-[(6-decyl-2-naphthoyl)amino]propanoyl}pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(5S)-6-[[(7S,10S,13S)-13-carboxy-3,18-dihydroxy-10-methyl-8,11-dioxo-9,12-diazatricyclo[13.3.1.12,6]icosa-1(19),2(20),3,5,15,17-hexaen-7-yl](methyl)amino]-6-oxo-5-[(3-{[4-(8-phenyloctyl)benzoyl]amino}propanoyl)amino]hexan-1-aminium;
(5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-[(3-{[(4′-nonylbiphenyl-4-yl)carbonyl]amino}propanoyl)amino]-6-oxohexan-1-aminium;
(5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-({3-[(6-decyl-2-naphthoyl)amino]propanoyl}amino)-6-oxohexan-1-aminium;
(8S,11S,14S)-3,18-dihydroxy-11-methyl-14-(methyl{[(2)-1-(3-{[(4′-nonylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(8S,11S,14S)-3,18-dihydroxy-11-methyl-14-(methyl{[(2)-1-(3-{[(4′-nonylbiphenyl-4-yl)carbonyl]amino}propanoyl)azetidin-2-yl]carbonyl}amino)-10,13-dioxo-9,12-diazatricyclo[13.3.1.1^{2,6}]icosa-1(19),2(20),3,5,15,17-hexaene-8-carboxylic acid; or
(8S,11S,14S)-3,18-dihydroxy-11-methyl-14-(methyl{[(2R)-1-{3-[(6-decyl-2-naphthoyl)amino]propanoyl}azetidin-2-yl]carbonyl}amino)-10,13-dioxo-9,12-diazatricyclo[13.3.1.1^{2,6}]icosa-1(19),2(20),3,5,15,17-hexaene-8-carboxylic acid.

### β-Lactam Antibiotics

The compounds of the invention may be used in combination with antibiotic agents for the treatment of bacterial infections. In accordance with the instant invention, it is generally advantageous to use a compound of formula I in admixture or conjunction with a carbapenem, penicillin, cephalosporin or other β-lactam antibiotic or prodrug. It also advantageous to use a compound of formula I in combination with one or more β-lactam antibiotics. In this case, the compound of formula I and the β-lactam antibiotic can be administered separately or in the form of a single composition containing both active ingredients.

Carbapenems, penicillins, cephalosporins and other β-lactam antibiotics are suitable for co-administration with the compounds of Formula I, whether by separate administration or by inclusion in the compositions according to the invention.

β-lactams antibiotics are characterized by a 4-membered β-lactam core of consisting of three carbon atoms and one nitrogen atom. β-lactams antibiotics include carbapenems, cephalosporins, monolactams and penicillins.

Due to the activity of β-lactamases, a β-lactam antibiotic may be degradaded. Thus, in certain embodiments of the invention, a suitable β-lactamase inhibitor, such as clavunic acid, sulbactam or tazobactam, may be administered, either together or separately, with the β-lactam antibiotic. See, e.g., Drawz et al., 2010, Clin Microbiol Rev 23:160-201. The β-lactamase inhibitor should preferably be available at the desired site of action before the antibiotic to ensure immediate protection of the antibiotic.

Carbapenems

Carbapenems are a class of β-lactam antibiotics that possess the carbapenem ring system (a four-member lactam ring fused to a five member thiazolidinic secondary ring through the nitrogen and adjacent tetrahedral carbon atom). Carbapenems tend to exhibit an extremely broad spectrum of activity against gram-positive and gram-negative aerobic and anaerobic species, which is partly due to its high stability in the presence of β-lactamases. They act by binding to penicillin-binding proteins.

Carbapenems include, but are not limited to, carbapenems (including 1β-methylcarbapenems) having a side chain at the 2 position, including, but not limited to, 2-substituted alkyl-3-carboxycarbapenems (See U.S. Pat. No. 5,021,565); 2-aryl carbapenems (See U.S. Pat. No. 6,277,843); 2-(aza-9-fluorenonyl)carbapenems (See U.S. Pat. No. 5,294,610) and 2-(9-fluorenonyl)-carbapenems (See U.S. Pat. Nos. 5,034,384 and 5,025,007) including 2-(fluoren-9-on-3-yl)carbapenems containing a (bis-quaternary ammonium)methyl moiety (See U.S. Pat. No. 5,451,579); 2-benzocoumarinyl-carbapenems (See U.S. Pat. Nos. 5,216,146; 5,182,384; 5,162,314; and 5,153,186); 2-biphenyl-carbapenems (See U.S. Pat. Nos. 5,350,846; 5,192,758; 5,182,385; 5,025,006; and 5,011,832); 2-carbolinyl derivatives (See U.S. Pat. No. 5,532,261); 2-(substituted-dibenzofuranyl and dibenzothienyl)carbapenems (See U.S. Pat. Nos. 5,240,920 and 5,025,008); halophenoxy substituted carbapenems (U.S. Pat. No. 6,310,055); carbapenems with cationic heteroaryl substituents (See U.S. Pat. No. 5,496,816); carbapenems having an externally alkylated mono- or bicyclic 2-quaternary heteroarylalkyl substituent (See U.S. Pat. No. 4,729,993) or carbapenems having an internally or externally alkylated mono- or bicyclic 2-quaternary heteroarylalkyl thiomethyl substituent (See U.S. Pat. No. 4,725,594); carbapenems having a 2-heteroaryliumaliphatic substituent (See U.S. Pat. No. 4,680,292); 2-naphthyl-carbapenems (See U.S. Pat. Nos. 5,006,519 and 5,032,587); 2-naphthosultam carbapenems (See U.S. Pat. Nos. 6,399,597; 6,294,529; 6,251,890; 6,140,318; 6,008,212; 5,994,345; 5,994,343; 5,756,725), e.g., 2-(naphthosultamyl)methyl-carbapenems (See U.S. Pat. No. 6,221,859), carbapenems substituted at the 2-position with a 1,1 dioxo-2,3-dihydro-naphtho[1,8-de][1,2]thiazin-2-yl group or 1,1,3 trioxo-2,3-dihydro-naphtho[1,8-de][1,2]thiazin-2-yl group linked through a CH₂ group (U.S. Pat. No. 6,346,526) or a 1,1-dioxo-2H-1-thia-2,3-diaza-naphthalene linked through a CH₂ group (See U.S. Pat. No. 6,346,525); 2-(N-imidazoliumphenyl)-carbapenems (See U.S. Pat. No. 5,276,149); 2-phenanthridinyl carbaphenems (See U.S. Pat. Nos. 5,336,674; 5,328,904; 5,214,139 and 5,153,185); 2-phenanthrenyl-carbapenems (See U.S. Pat. Nos. 5,177,202; 5,004,740 and 5,004,739); 2-phenanthridonyl-carbapenems including 2-phenanthridonyl carbapenems having cationizeable substituents (See U.S. Pat. No. 5,157,033); 2-phenyl-carbapenems (See U.S. Pat. Nos. 5,334,590 and 5,256,777), including 2-(heteroaryliumalkyl)phenyl carbapenems (See U.S. Pat. No. 4,978,659), 2-(heteroarylsubstituted)phenyl carbapenems (See U.S. Pat. No. 5,034,385), 2-(heterocyclylalkyl)phenyl carbapenems (See U.S. Pat. Nos. 5,247,074; 5,037,820 and 4,962,101), 2-(heterocyclylheteroaryliumalkyl)phenyl carbapenems (See U.S. Pat. No. 5,362,723), 2-heteroarylphenyl-carbapenems (See U.S. Pat. Nos. 5,143,914 and 5,128,335) including cationic 2-heteroarylphenyl-carbapenems (See U.S. Pat. No. 5,342,933), 2-iodo-substituted phenyl (See U.S. Pat. No. 6,255,300); (N-pyridiniumphenyl)-carbapenems (See U.S. Pat. No. 5,382,575), triazolyl and tetrazolyl phenyl substituted carbapenems (See U.S. Pat. No. 5,350,746) including 2-(1,2,3-triazolyisubstituted)phenyl carbapenems (See U.S. Pat. No. 5,208,229), and 2-(quinoliniumalkyl and isoquinoliniumalkyl)phenyl carbapenems (See U.S. Pat. Nos. 5,124,323 and 5,055,463); 2-(2-substituted pyrrolidin-4-yl)thio-carbapenems (See U.S. Pat. Nos. 5,756,765 and 5,641,770); 2-(3-pyridyl)-carbapenems (See U.S. Pat. No. 5,409,920); 2-(unsubstituted or carbon-substituted)-1-carbapen-2-em-3-carboxylic acid derivatives (See U.S. Pat. Nos. 5,258,509 and 4,775,669); carbapenems substituted at the 2-position with a 9,9-dioxo-10H-9-thia-10-aza-phenanthrene linked through a CH₂ group (See U.S. Pat. No. 6,294,528); carbapenems substituted at the 2-position with fused bi- and tricyclic 2,2-dioxo-3-X-2-thia-1-aza-cyclopenta ring systems linked through a CH₂ group (See U.S. Pat. Nos. 6,291,448 and 6,265,395); and carbapenems substituted at the 2-position with a 2-mercaptobenzothiazole moiety linked through a group —Z—CH₂— where Z represents an trans-ethenediyl group, ethynediyl group or is absent (See U.S. Pat. No. 6,288,054).

Carbapenems also include, but are not limited to, 3-phosphonate carbapenems (See U.S. Pat. No. 4,565,808); 6-amido-carbapenems (See U.S. Pat. No. 5,183,887), including, but not limited to, 6-amido-1-methyl carbapenems (See U.S. Pat. No. 5,138,050) and 6-amido-1-methyl-2-(substituted-thio)carbapenems (See U.S. Pat. No. 5,395,931); bridged carbapenems including bridged biphenyl carbapenems (See U.S. Pat. Nos. 5,401,735; 5,384,317; 5,374,630; 5,372,993); cyclic amidinyl and cyclic guanidinyl thio carbapenems (See U.S. Pat. No. 4,717,728); and tricyclic carbapenem compounds (See U.S. Pat. Nos. 6,284,753 and 6,207,823; International Publication No. WO92/03437).

Carbapenems also include, but are not limited to, 1β-methylcarbapenem derivatives (See U.S. Pat. Nos. 7,001,897; 6,479,478; 5,583,218; 5,208,348; 5,153,187; International Patent Publication Nos. WO 98/34936 and WO 99/57121; Japanese patent publication 2-49783, Japanese patent publication 8-53453); carbapenems with a carboxy substituted phenyl group (See U.S. Pat. No. 5,478,820); carbapenem derivatives having a substituted imidazo[5,1-b]thiazole group at the 2-position on the carbapenem ring (See U.S. Pat. Nos. 6,908,913; 6,680,313; 6,677,331; International Publication Nos. WO 98/32760 and WO 00/06581), a substituted phenyl or a substituted thienyl directly substituted at position 3 of 7-oxo-1-azabicyclo[3.2.0]hept-2-ene (see U.S. Pat. No. 7,205,291). Carbapenems also include, but are not limited to, those disclosed in U.S. Pat. Nos. 4,943,569 and 4,888,344.

Examples of carbapenems that may be used with a compound of the present invention include, but are not limited to, imipenem, meropenem, biapenem, (4R,5S,6S)-3-[3S,5S)-5-(3-carboxyphenyl-carbamoyl)pyrrolidin-3-ylthio]-6-(1R)-1-hydroxyethyl]-4-methyl-7-oxo-1-azabicyclo[3.2.0]hept-2-ene-2-carboxylic acid, (1S,5R,6S)-2-(4-(2-(((carbamoylmethyl)-1,4-diazoniabicyclo[2.2.2]oct-1-yl)-ethyl (1,8-naphthosultam)methyl)-6-[1(R)-hydroxyethyl]-1-methyl carbapen-2-em-3-carboxylate chloride, BMS181139 ([4R-[4alpha,5beta,6beta(R*)]]-4-[2-[(aminoiminomethyl)amino]ethyl]-3-[(2-cyanoethyl)thio]-6-(1-hydroxyethyl)-7-oxo-1-azabicyclo[3.2.0]hept-2-ene-2-carboxylic acid), BO2727 ([4R-3[3S*,5S*(R*)], 4alpha,5beta,6beta(R*)]]-6-(1-hydroxyethyl)-3-[[5-[1-hydroxy-3-(methylamino)propyl]-3-pyrrolidinyl]thio]-4-methyl-7-oxo-1-azabicyclo[3.2.0]hept-2-ene-2-carboxylic acid monohydrochloride), E1010 ((1R,5S,6S)-6-[1(R)-hydroxymethyl]-2-[2(S)-[1(R)-hydroxy-1-[pynolidin-3(R)-yl]methyl]pyrrolidin-4(S)-ylsulfanyl]-1-methyl-1-carba-2-penem-3-carboxyl is acid hydrochloride), S4661((1R,5S,6S)-2-[(3S,5S)-5-(sulfamoylaminomethyl)pyrrolidin-3-yl]thio-6-[(1R)-1-hydroxyethyl]-1-methylcarbapen-2-em-3-carboxylic acid) and (1S,5R,6S)-1-methyl-2-{7-[4-(aminocarbonylmethyl)-1,4-diazoniabicyclo(2.2.2)octan-1-yl]-methyl-fluoren-9-on-3-yl}-6-(1R-hydroxyethyl)-carbapen-2-em-3-carboxylate chloride. Preferred carbapenems include, but are not limited to, biapenem, doripenem, ertapenem, imipenem, meropenem, panipenem, and tebipenem. (4R,5S,6S)-3-[(3S,5S)-5-(3-carboxyphenylcarbamoyl)pyrrolidin-3-ylthio]-6-(1R)-1-hydroxyethyl]-4-methyl-7-oxo-1-azabicyclo[3.2.0]hept-2-ene-2-carboxylic acid.

Carbapenems also includes pharmaceutically acceptable salts, esters and hydrates of the compounds described above.

Carbapenems may be crystallized or recrystallized from solvents such as organic solvents forming solvates. This invention includes within its scope stoichiometric solvates including hydrates as well as compounds containing variable amounts of solvents such as water that may be produced by processes such as lyophilization. Carbapenems may be prepared in crystalline form by for example dissolution of the compound in water, preferably in the minimum quantity thereof, followed by admixing of this aqueous solution with a water miscible organic solvent such as a lower aliphatic ketone such as a di-(C₁₋₆) alkyl ketone, or a (C₁₋₆) alcohol, such as acetone or ethanol. Crystalline forms of carbepenems may also be synthesized as disclosed in U.S. Pat. No. 7,145,002.

Synthesis of carbapenems is well known in the art and is disclosed in the patents and patent applications in this section.

Cephalosporins/Cephamycins

Cephalosporins contain a nucleus of a β-lactam ring and a 6 member dihydrothiazine ring. Cephamycins contain an additional methoxy group on the 13-lacctam ring. Cephalosporins and cephamycines often have activity against Gram-positive or Gram-negative organisms, but are not typically active against both.

Exemplary cephalosporins include, but are not limited to, 4-hydroxycephalexin, cefaclor, cefadroxil, cefadyl, cefalexin, cefamandole, cefatrizine, cefazolin, cefditoren, cefepime, cefetarnet, cefdinir, cefinetazole, cefixime, cefizox, cefotaxime, cefinetazole, cefobid, cefonicid, cefoperazone, cefotan, cefotaxime, cefotetan, cefoxitin, cefpirome, cefpodoxime, cefprozil, cefradine, cefsulodin, ceftazidime, ceftibuten, ceftidoren, ceftin, ceftizoxime, ceftriaxone, cefuroxime, cefuroxime axetil, cephalexin, cefzil, cephacetrile, cephaloglycin, cephaloridine, cephalothin, cephamandole nafate, cephapirin, cephradine, and other known cephalosporins, all of which may be used in the form of pro-drugs thereof, pharmaceutically acceptable salts thereof or pharmaceutically acceptable derivatives thereof. Examples for pharmaceutically acceptable cephalosporin derivatives, which may be used in the delivery system of the invention, are cefpodoxime proxetil and cefuroxime axetil. FK-037, 5-amino-2-[[(6R,7R)-7-[[(Z)-2-(2-amino-4-thiazolyl)-2-methoxyimino) acetyl]amino]-2-carboxy-8-oxo-5-thia-1-azabicyclo[4.2.0]oct-2-en-3-yl]methyl]-1-(2-hydroxyethyl)-1H-pyrazolium hydroxide, inner salt, sulfate (1:1).

Particularly suitable cephalosporins for co-administration with the compounds according to the invention include cefotaxime, ceftriaxone and ceftazidime, which may be used in the form of their pharmaceutically acceptable salts, for example their sodium salts.

Penicillins

Penicillins are a class of β-lactam antibiotics that possess a β-lactam, ring and a thiazolidine ring. Penicillins are used to treat susceptible, usually Gram-positive, organisms.

Exemplary penicillins include, but are not limited to, amoxicillin, amoxycillin, amoxicillin-clavulanate, ampicillin, azidocillin, azlocillin, benzathine penicillin, benzylpenicillin (penicillin g), carbenicillin, carboxypenicillin, cloxacillin, co-arnoxiclav, cyclacillin, dicloxacillin, epicillin, flucloxacillin, hetacillin, mezlocillin, nafcillin, oxacillin, phenoxymethylpenicillin v), piperacillin, pirbenicillin, pivmecillinam, procaine benzylpenicillin (procaine penicillin), propicillin, sulbenicillin, tazocin (ureidopenicillin piperacillin with the beta-lactamase inhibitor tazobactam), ticarcillin, timentin, and other known penicillins, or pharmaceutically acceptable salts thereof. Such penicillins may be used in the form of their pharmaceutically acceptable salts, for example their sodium salts.

The penicillins may be used in the form of pro-drugs thereof; for example as in vivo hydrolysable esters, for example the acetoxymethyl, pivaloyloxymethyl, α-ethoxycarbonyloxy-ethyl and phthalidyl esters of ampicillin, benzylpenicillin and amoxycillin; as aldehyde or ketone adducts of penicillins containing a 6-α-aminoacetamido side chain (for example hetacillin, metampicillin and analogous derivatives of amoxycillin); and as α-estsers of carbenicillin and ticarcillin, for example the phenyl and indanyl α-esters.

Alternatively, ampicillin or amoxycillin may be used in the form of fine particles of the zwitterionic form (generally as ampicillin trihydrate or amoxycillin trihydrate) for use in an injectable or infusable suspension, for example, in the manner described herein in relation to the compounds of formula I. Amoxycillin, for example in the form of its sodium salt or the trihydrate, is particularly preferred for use in compositions according to the invention.

Monobactams

Monobactams have a single β-lactam core. Aztreonam is currently the only example of a monobactam.

Example of β-lactam antibiotics other than those described above that may be co-administered with the compounds according to the invention is latamoxef (Moxalactam™).

### Pharmaceutical Applications

The present invention provides methods of treating bacterial infections in a patient in need thereof which comprises administering a therapeutically effective amount of a compound of formula I. In certain embodiments, the present invention provides methods of treating bacterial infections in a patient in need thereof which comprises administering a therapeutically effective amount of a compound of formula I in combination with a β-lactam antibiotic. The β-lactam antibiotic may be a carbapenem, cephalosporin/cephamycin, monolactam or penicillin. In certain embodiments, a β-lactamase inhibitor may also be administered. In embodiments where the β-lactam antibiotic is a carbapenem, the method may further comprise administering a DHP inhibitor. The present invention also provides pharmaceutical compositions that can be used for therapeutic treatments. As used herein, a patient may be a mammal, e.g., a dog, cat, horse, pig, or primate. The patient may also be an adult or child. Preferably, the patient is an adult human or human child.

As used herein, “pharmacologically effective amount” or “therapeutically effective amount” generally refers to the amount of a compound of formula I (or alternatively, the amount of a compound of formula I and a β-lactam antibacterial agent, in combination) which results in the inhibition of the normal metabolism of bacterial cells causing or contributing to a bacterial infection. In therapeutic applications, the methods and compositions of the invention are used for administration to a patient already suffering from an infection from bacteria, in an amount sufficient to cure or at least partially arrest the symptoms of the infection. Amounts effective for this use will depend on the severity and course of the infection, previous therapy, the patient's health status and response to the drugs, and the judgment of the treating physician. Therapeutically effective amounts can be assessed by clinical trial results and/or model animal infection studies.

Organisms amenable to therapy by way of the methods and compositions disclosed herein include Gram-positive bacteria such as Enterococcus faecalis, Staphylococcus aureus, Staphylococcus epidermidis, including methicillin resistant strains and (b) Gram-negative bacteria such as Haemophilus influenzae, Pseudomonas aeruginosa, and Klebsiella pneumoniae.

Bacterial infections treatable with the methods and compositions of the invention include, but are not limited to, complicated intra-abdominal infection, appendicitis, acute pelvic infections, complicated urinary tract infections, complicated skin and skin structure infections, diabetic foot ulcer, community-acquired pneumonia, nosocomial pneumonia, acute pulmonary excerbations in cystic fibrosis patients, febrile neutropenia, lower respiratory infections, bacterial septicemia, bone and joint infection, endocarditis, polymicrobial infection, and bacterial meningitidis. See Zhanel et al., 2007, Drugs 67:1027-1052 and Dalhoff et al., 2006, Biochem Pharmacol 71:1085-1095.

In some embodiments of the invention, due to the synergistic effects of the potentiator and a β-lactam (e.g., carbapenem), the dosage of the β-lactam can be lower than a β-lactam used alone. The dosage of β-lactam in a combination regimen may be ½, ⅓, ¼, ⅕, ⅙, ⅛, or 1/10 of the dosage of the β-lactam used alone.

In the various embodiments of the invention, the β-lactam antibiotic and a compound of formula I can be administered sequentially or concurrently. Preferably, the β-lactam antibiotic and compound of formula I are administered together. When administered concurrently, the β-lactam antibiotic and compound of formula I may be administered in the same formulation or in separate formulations. When administered sequentially, either the β-lactam or compound of formula I may be administered first. After administration of the first compound, the other compound is administered within 1, 2, 3, 4, 5, 10, 15, 30, or 60 minutes. In one aspect of the invention, when a DHP inhibitor is used with a carbapenem, it may be administered separately, or in a formulation with a potentiator and/or carbapenem.

Once improvement of the patient's conditions has occurred, a maintenance dose is administered if necessary. Subsequently, the dosage or the frequency of administration, or both, can be reduced, as a function of the symptoms, to a level at which the improved condition is retained. When the symptoms have been alleviated to the desired level, treatment can cease. Patients can, however, require intermittent treatment on a long-term basis upon any recurrence of the disease symptoms.

### Pharmaceutical Compositions

In one embodiment of the invention, when the compound of formula I is to be used alone, the compound of formula I may be in a composition, e.g., a pharmaceutical composition, containing the compound of formula I and a pharmaceutically acceptable carrier or excipient. In another embodiment of the invention, a composition, e.g., a pharmaceutical composition, comprises, or consists essentially of, a compound of formula I and a pharmaceutically acceptable carrier or excipient. The compound of formula I is in such amounts that it constitutes a pharmaceutically or therapeutically effective dose or amount. The compounds can be prepared as pharmaceutically acceptable salts (i.e., non-toxic salts which do not prevent the compound from exerting its effect).

In another embodiment of the invention, when the compound of formula I and a β-lactam are administered separately, the compound of formula I and/or β-lactam may be in a composition, e.g., a pharmaceutical composition, containing the compound of formula I or β-lactam and a pharmaceutically acceptable carrier or excipient. In another embodiment of the invention, a composition, e.g., a pharmaceutical composition, comprises, or consists essentially of, a compound of formula I, a β-lactam, and a pharmaceutically acceptable carrier or excipient. Accordingly, formulations of the present invention may contain a compound of formula I together with a β-lactam and one or more pharmaceutically or therapeutically acceptable carriers or excipients. The compound of formula I and β-lactam are in such amounts and relative proportion that the combination constitutes a pharmaceutically or therapeutically effective dose or amount. The compounds can be prepared as pharmaceutically acceptable salts (i.e., non-toxic salts which do not prevent the compound from exerting its effect).

In certain embodiments, a composition of the invention further comprises a β-lactamase inhibitor.

Pharmaceutically acceptable carriers or excipients can be used to facilitate administration of the compound, for example, to increase the solubility of the compound. Solid carriers include, e.g., starch, lactose, dicalcium phosphate, microcrystalline cellulose, sucrose, and kaolin, and optionally other therapeutic ingredients. Liquid carriers include, e.g., sterile water, saline, buffers, polyethylene glycols, non-ionic surfactants, and edible oils such as corn, peanut and sesame oils, and other compounds described e.g., in the MERCK INDEX, Merck & Co., Rahway, N.J. In addition, various adjuvants such as are commonly used in the art may be included. For example: flavoring agents, coloring agents, preservatives, and antioxidants, e.g., vitamin E, ascorbic acid, BHT and BHA. Various other considerations are described, e.g., in Gilman et al. (eds) (1990) Goodman and Gilman's: The Pharmacological Basis of Therapeutics, 8th Ed., Pergamon Press. Methods for administration are discussed therein, e.g., for oral, sublingual, intravenous, intraperitoneal, or intramuscular administration, subcutaneous, topically, and others.

The pharmaceutical compositions described herein may be presented in a number of appropriate dosage forms; e.g., tablets, capsules, pills, powders, suspensions, solutions, and the like, for oral administration; solutions, suspensions, emulsions, and the like, for parenteral administration; solutions for intravenous administration; and ointments, transdermal patches, and the like, for topical administration. The preferred form depends on the intended mode of administration and therapeutic application. For some compounds a pharmacologically acceptable salt of the compound will be used to simplify preparation of the composition. The compounds may be employed in powder or crystalline form, in liquid solution, or in suspension.

Compositions for injection, a preferred route of delivery, may be prepared in unit dosage form in ampules, or in multidose containers. The injectable compositions may take such fowls as suspensions, solutions, or emulsions in oily or aqueous vehicles, and may contain various formulating agents. Alternatively, the active ingredient may be in powder (lyophillized or non-lyophillized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

Compositions intended for oral use may be prepared according to methods known to the art for the manufacture of pharmaceutical compositions and such compositions may contain one or more agents selected from the group consisting of sweetening agents, flavoring agents, coloring agents and preserving agents in order to provide pharmaceutically elegant and palatable preparation. See, e.g., Remington: The Science and Practice of Pharmacy, 21^{st} ed., Lippincott Williams & Wilkins, 2005. Tablets containing the active ingredient in admixture with non-toxic pharmaceutically acceptable excipients may also be manufactured by known methods. The excipients used may be for example, (1) inert diluents such as calcium carbonate, sodium carbonate, lactose, calcium phosphate or sodium phosphate; (2) granulating and disintegrating agents such as corn starch, or alginic acid; (3) binding agents such as starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,160,452; and 4,265,874 to form osmotic therapeutic tablets for controlled release.

In some cases, formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, for example peanut oil, liquid paraffin, or olive oil.

Aqueous suspensions normally contain the active materials in admixture with excipients suitable for the manufacture of aqueous suspensions. Such excipients may be suspending agents such as sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived from a fatty acid and a hexitol such as polyoxyethylene sorbitol monooleate, or a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents such as sucrose or saccharin.

Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be preserved by the addition of an antioxidant such as ascorbic acid.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions of the invention may also be in the form of oil-in-water emulsions. The oily phase may be a vegetable oil such as olive oil or arachis oils, or a mineral oil such as liquid paraffin or a mixture thereof. Suitable emulsifying agents may be (1) naturally-occurring gums such as gum acacia and gum tragacanth, (2) naturally-occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a demulcent, a preservative and flavoring and coloring agents.

When a carbapenem is used, the pharmaceutical compositions described above may be combined or used with dehydropeptidase (DHP) inhibitors. Many carbapenems are susceptible to attack by the renal enzyme DHP. This attack or degradation may reduce the efficacy of the carbapenem. Many carbapenems, on the other hand, are less subject to such attack, and therefore may not require the use of a DHP inhibitor. However, such use is optional and contemplated to be part of the present invention. Inhibitors of DHP and their use with carbapenems are disclosed in, e.g., U.S. Pat. No. 5,071,843 and European Patent Nos. EP 0 007 614 and EP 0 072 014. When a DHP inhibitor is used with a pharmaceutical invention described above, the DHP inhibitor may be in a pharmaceutical composition with a pharmaceutically acceptable carrier or excipient. A preferred DI-IP inhibitor is 7-(1,2-amino-2-carboxyethylthio)-2-(2,2-dimethylcyclopropanecarboxamide)-2-heptenoic acid, also known as cilastin, or a useful salt thereof.

In one aspect of the invention, the combination of the DHP inhibitor and the carbapenem can be in the form of a pharmaceutical composition containing the two compounds in a pharmaceutically acceptable carrier. The two can be employed in amounts so that the weight ratio of the penem to inhibitor is 1:3 to 30:1, and preferably 1:1 to 5:1. A preferred weight ratio of carbapenem:DHP inhibitor in the combination compositions is about 1:1.

In certain aspects of the invention, pharmaceutical compositions of the present invention contemplate a compound of formula I in combination with a carbapenem, a DHP inhibitor such as, cilastatin, and a pharmaceutically acceptable carrier.

### Dosage and Administration

The pharmaceutical compositions of the invention can be administered parenterally (intravenously or intramuscularly), or subcutaneously, particularly when they are used in combination with a β-lactam antibiotic or comprise a β-lactam antibiotic. They may also be administered orally or sublingually. The compounds of this invention may also be used to treat topical antibacterial infection.

The amount of active ingredients that may be combined with the carrier materials to produce a single dosage form will vary depending upon the host treated and the particular mode of administration. Without being bound by any theory, it is believed that the combination of a compound of formula I and a β-lactam will reduce the required dosage for the β-lactam and/or the compound of formula 1.

In cases where the active ingredients are not combined, i.e., are administered separately, the β-lactam antibiotic and compound of formula I are preferably administered on a schedule consistent with concurrent administration or administration within 1, 2, 3, 4, 5, 10, 15, 20, 25, or 30 minutes of each other.

Exemplary intravenous or intramuscular dosages of a compound of formula I are in the range of 50 mg to 2 g, 100 mg to 1 g, 250 mg to 750 mg. Other exemplary dosages for intravenous or intramuscular use include ranges of 0.1 to 200, 0.2 to 100, 0.5 to 50, or 1 to 25 mg/kg/day. Preferably, the dosage is given 1, 2, 3 or 4 times daily.

β-lactam antibiotics may suitably be administered to the patient at a daily dosage of from 0.1 to 200, 0.2 to 100, 0.5 to 75, 0.7 to 50, 1 to 25, or 5 to 20 mg/kg of body weight. About 5 to 50 mg of a β-lactam per kg of body weight is preferred. Preferably, the dosage is given 1, 2, 3 or 4 times daily. For instance, the β-lactam can be administered intramuscularly or intravenously in amounts of 1-100 mg/kg/day, preferably 1-20 mg/kg/day, or 1-5 mg/kg/dose, in divided dosage forms, e.g., 1, 2, 3 or 4 times daily.

For an adult human (of approximately 70 kg body weight), from 50 to 3000 mg, preferably from 100 to 1000 mg, of a β-lactam may be administered daily, suitably in from 1, 2, 3, 4, 5, or 6 separate doses. When the β-lactam are presented in unit dosage form, each unit dose may suitably comprise from about 25 to about 1000 mg, preferably about from 50 to about 500 mg, of a β-lactam. Each unit dose may, for example, be 62.5, 100, 125, 150, 200 or 250 mg of a β-lactam. The preferred dosage is 250 mg to 1000 mg of the antibacterial given one to four times per day. More specifically, for mild infections a dose of about 250 mg two or three times daily is recommended. For moderate infections against highly susceptible gram positive organisms a dose of about 500 mg three or four times daily is recommended. For severe, life-threatening infections against organisms at the upper limits of sensitivity to the antibiotic, a dose of about 1000-2000 mg three to four times daily may be recommended.

For children, a dose of about 1 to 100, 2.5 to 50, 5 to 25, or 10 to 20 mg/kg of body weight is preferred; a dose of 10 mg/kg is typically recommended. Preferably the dosage is given 1, 2, 3, or 4 times per day. Unit dosages may be as used for adults.

The compositions for human delivery per unit dosage may contain from about 0.01% to as high as about 99% of active material, the preferred range being from about 10-60%. The composition will generally contain from about 15 mg to about 2.5 g of the active ingredient; however, in general, it is preferable to employ dosage amounts in the range of from about 250 mg to 1000 mg. In parenteral administration, the unit dosage will typically include the pure compound in sterile water solution or in the form of a soluble powder intended for solution, which can be adjusted to neutral pH and isotonic.

When the compound of formula I is co-administered with a β-lactam, the ratio of the compound of formula I to β-lactam may vary within a wide range. The ratio may, for example, be from 100:1 to 1:100; more particularly, it may, for example, be from 2:1 to 1:30. The amount of β-lactam according to the invention will normally be approximately similar to the amount in which it is conventionally used.

The dosage to be administered depends to a large extent upon the condition and size of the subject being treated, the route and frequency of administration, the sensitivity of the pathogen to the particular compound selected, the virulence of the infection and other factors. Such matters, however, are left to the routine discretion of the physician according to principles of treatment well known in the antibacterial arts. Another factor influencing the precise dosage regimen, apart from the nature of the infection and peculiar identity of the individual being treated, is the molecular weight of the compound.

In embodiments where a β-lactamase inhibitor is used, the molar β-lactam antibiotic to β-lactamase inhibitor ratio is from 2:1 to 18:1, preferably from 2:1 to 4:1.

In one aspect of the invention, a DHP inhibitor is also administered either sequentially or concurrently from the compound of formula I and/or carbapenem. The DHP inhibitor can be administered, orally, intramuscularly, or IV, in amounts of 1-100 mg/kg/day, or preferably 1-30 mg/kg/day, or 1-5 mg/kg/dose and may be in divided dosage forms, e.g., three or four times a day.

One preferred dosage regimen and level is the combination of the compound 2-[3S)-1-acetimidoyl-pyrollidin-3-yl-thio]-6-(1-hydroxyethyl)-carbapen-2-e m-3-sodium carboxylate and the crystalline form of 74-amino-2-carboxyethylthio)-2-(2,2-dimethylcyclopropanecarboxamido)-2-heptenoic acid, co-administered in a sterile aqueous IV injection form (sodium salt), at a level of 250 or 500 mg of the penem compound and about 1:1 (weight) of the heptenoic acid, or 250 or 500 mg. This dose can be given to humans (each assumed to weigh about 80 kg.) from 1 to 4 times daily, that is 3.1-25 mg/kg/day of each drug. This carbapenem can also be combined with inhibitor, ±Z-2-(2,2-dimethylcyclopropanecarboxamido)-2-ocetenoic acid and both administered parenterally, at dose levels (estimated for humans) at 2-8 mg/kg/dose of the carbapenem and 1-8 mg/kg/dose of the inhibitor, such doses being administered 1-4 times a day. The potentiator may be added at the dosages described above.

### Methods of Synthesis

The compounds of structural formula I can be prepared according to the procedures of the following Schemes, using appropriate materials and are further exemplified by the following specific Examples. The compounds illustrated in the Examples are not, however, to be construed as forming the only genus that is considered as the invention. The Examples further illustrate details for the preparation of the compounds of the present invention. Those skilled in the art will readily understand that known variations of protecting groups, of reagents, as well as of the conditions and processes of the following preparative procedures, can be used to prepare these compounds. It is also understood that whenever a chemical reagent such as an isocyanate, a boronic acid, or a boronate is not commercially available, such a chemical reagent can be readily prepared following one of numerous methods described in the literature. All temperatures are degrees Celsius unless otherwise noted. Mass spectra (MS) were measured either on electrospray ion-mass spectroscopy (ESMS)

The synthesis of the key macrocyclic intermediate XIII, exemplified in Scheme 1 and 2, is a modification of the procedures described by Tucker et al., 2007, J. Am. Chem. Soc. 129:15830-15838. The fully functionalized amino boronate ester V was prepared in 5 steps. N-protection of 1, —(S) tyrosine followed by methylation of the phenolic residue afforded the intermediate II. Ortho-directed iodination and subsequent cross coupling with pinacole diborane led to the boronate ester IV. Hydrogenolysis of the benzyl carbamate IV resulted in the isolation of the desired key building block V. The dipeptide IX was also prepared in 5 steps. N-BOC protection of the commercially available L-(S)-4-hydroxy phenyl alanine followed by EDCI/HOBt mediated coupling of the resulting acid VI with the methyl ester of L-alanine afforded the phenol VII. Methylation of the latter and subsequent ortho-directed iodination and saponification of the resulting ester afforded the second building block IX.

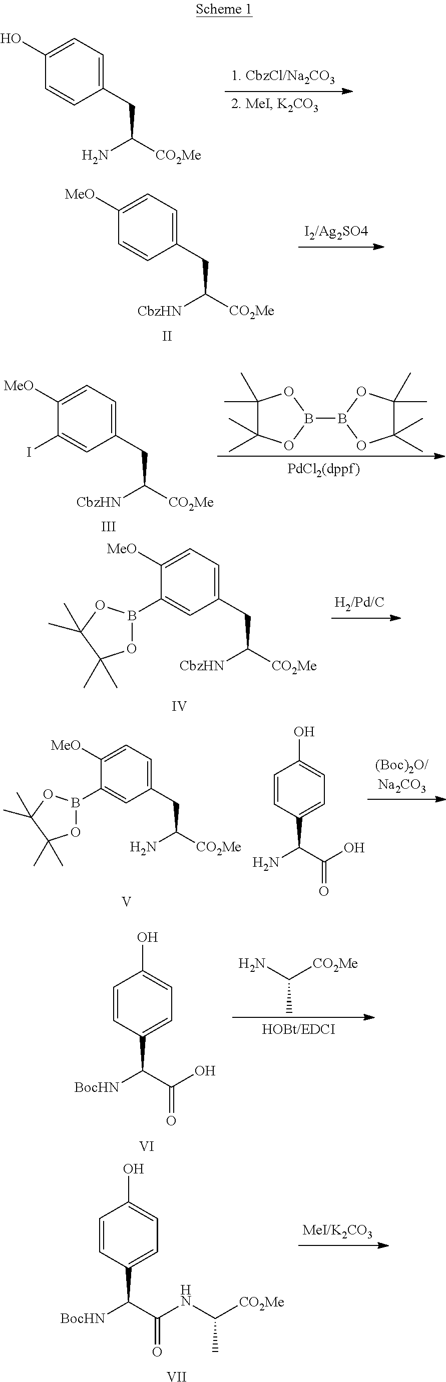

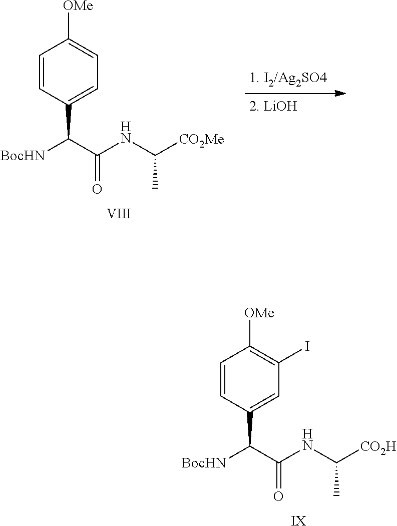

The macrocyclic core XIII was prepared in 6 steps from the two previously described building blocks. The amine V and the acid IX were coupled (EDCI/HOBt) to yield the tripeptide X. The macrocyclisation was performed via an intramolecular palladium catalyzed cross-coupling. The reaction was performed at high dilution (0.07M) to avoid intermolecular cross-coupling. Removal of the BOC protecting group and subsequent sulfonylation of the free amine afforded the nosyl derivative XII. N-methylation of the sulfonamide moiety followed by removal of the nosyl group yielded the desired amine XIII.

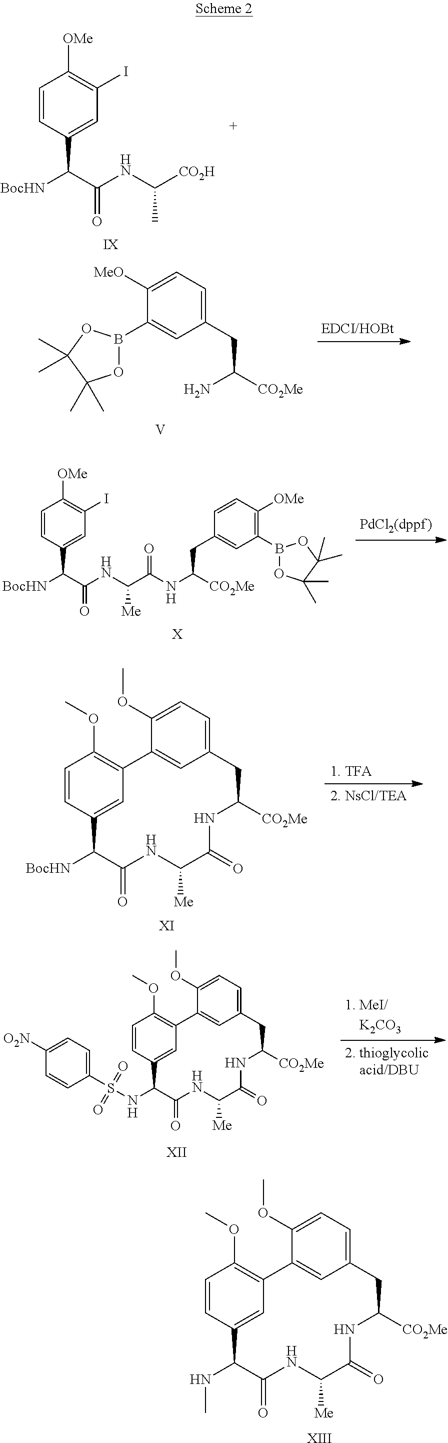

Intermediates described in Scheme 3 were prepared as follows and were subsequently coupled to the macrocyclic core XIII (see Scheme 4). The acid XIX was prepared in 4 steps from the aryl carboxylic acid XIV. Acid XIV can be coupled with methyl 3-aminopropanoate using EDCI/HOBt or HATU as the coupling agent. The resulting ester XV was saponified in presence of LiOH or NaOH prior to coupling under previously described conditions, with the amine XVII to afford the dipeptide XVIII. The latter was saponified affording the desired intermediate XIX. The intermediate XXI was prepared in similar fashion in two steps from the Cbz protected methyl 3-aminopropanoate.

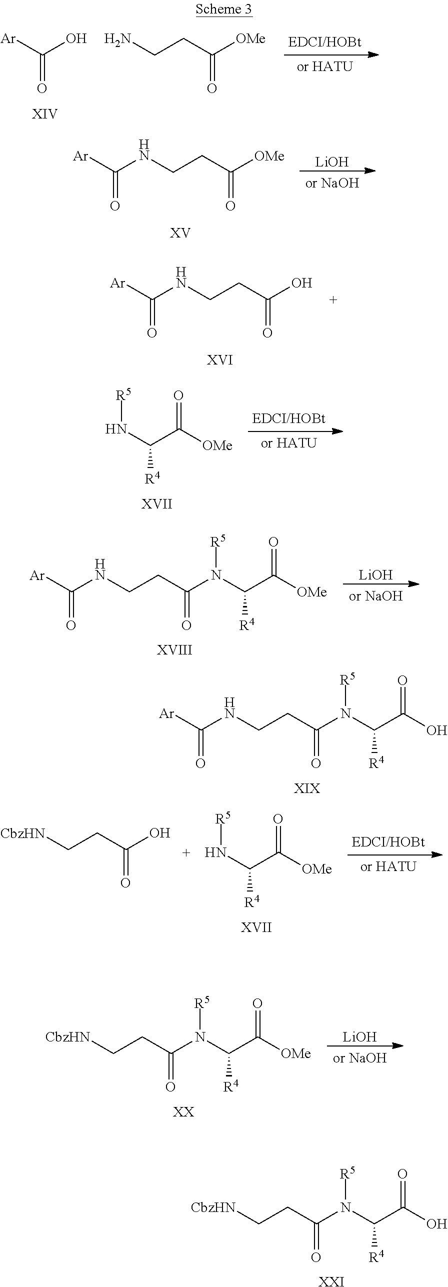

Examples described herein were prepared following three general methods (Scheme 4; A, B and C). They differentiate by the sequence of amide bond formation utilizing the macrocyclic core XIII and the intermediates described in Scheme 3, Method C was the preferred one, as it would prevent epimerization of the R₄ residue. In Method A, the macrocyclic core XIII was first coupled (EDCI/HOBt or HATU) with the acid XXI, followed by removal of the benzyloxy carbonyl protecting group by hydrogenolysis. The resulting amine was then coupled with the acid XIV to afford the desired compound I after removal of various protecting groups if required. In Method B, the core XIII was coupled with XIX to afford directly the desired compound I. Finally in Method C, the core was first coupled with the acid XXII, and the resulting amide XXIII was deprotected to afford the amine XXIV. The latter was coupled with the intermediate XVI to afford the desired compound I. Removal of various protecting groups completed the sequence of reaction steps. Carboxylic acids (R¹) were obtained via the hydrolysis of the corresponding ester using LiOH or NaOH, while BOC-amines (R^{4,5}) were deprotected using TFA. In some cases, AlBr₃/propanethiol mediated deprotection of the methyl ethers R² and R³ also led to the removal of BOC protective groups and hydrolysis of esters.

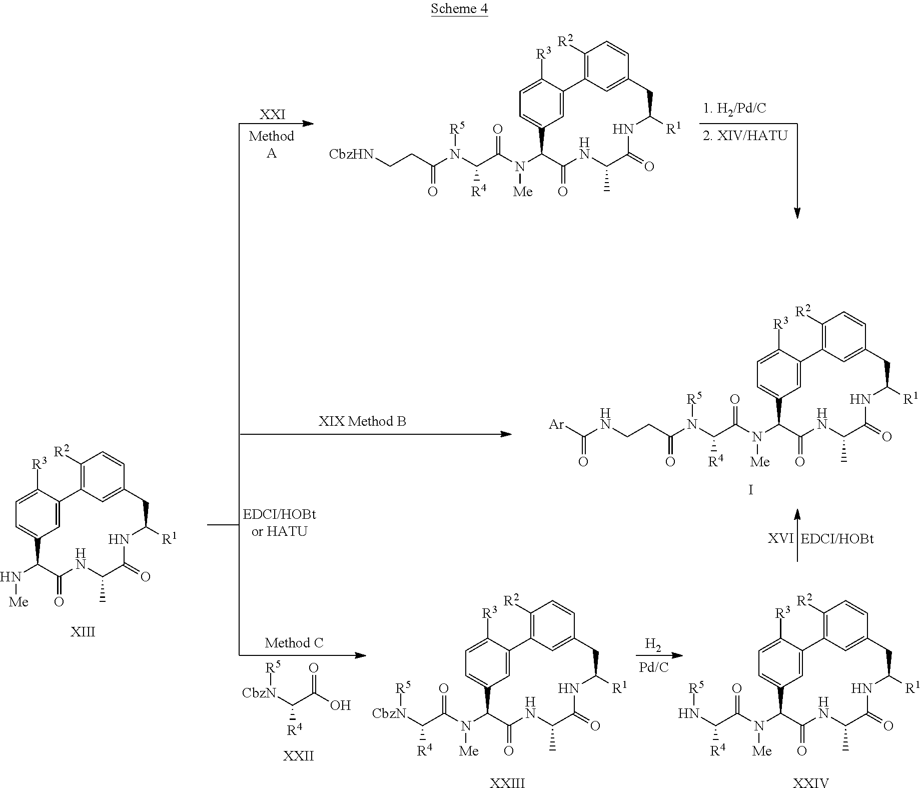

The specific embodiments described herein are offered by way of example only, and the invention is to be limited only by the terms of the appended claims along with the full scope of equivalents to which such claims are entitled. Indeed various modifications of the invention, in addition to those shown and described herein will become apparent to those skilled in the art from the foregoing description and accompanying drawings. Such modifications are intended to fall within the scope of the appended claims.

### ABBREVIATIONS

AlBr₃ Aluminium tribromide
BOC tert-Butyloxycarbonyl
Cbz Benzyloxycarbonyl (also CBz)
CH₃CN Acetonitrile
DBU 1,8-Diazabicyclo(5.4.0)undec-7-ene
DIPEA N,N-Diisopropylethylamine
DMF Dimethylformamide
DMSO Dimethylsulfoxide
EDCI 1-Ethyl-3-(3-dimethylaminopropyl)carbodiimide
EtOH Ethanol
EtOAc Ethyl acetate
Et₂O Diethyl ether
HATU 0-(7-Azabenzotriazol-1-yl)-1,1,3,3-tetramethyl-uronium hexafluorophosphate
Hex Hexane
HOBt 1-hydroxy-benzotriazole
HPLC High pressure liquid chromatography
KOAc Potassium acetate
LCMS Liquid chromatography I mass spectrometry
LiBH₄ Lithium borohydride
LRMS Low resolution mass spectrometry
MeOH Methanol
NEt₃ Triethylamine
Pd/C Palladium on activated carbon
Pd(PPh₃)₄ Tetrakis(triphenylphosphine)palladium(0)
PdCl₂(dppf) Palladium dichloride 1,3-bis(diphenylphosphino)propane
Tf₂O Triflic anhydride
TEA Triethylamine
TFA Trifluoroacetic acid
THF Tetrahydrofuran
Tol Toluene

### EXAMPLES

The invention is further described in connection with the following non-limiting examples. Unless other indicated, compounds described in the following examples which contain a basic amine group were isolated as trifluoroacetic acid salts. Conversion to the parent free amine may be accomplished by standard methods known in the art (e.g., neutralization with an appropriate organic base such as NaHCO3). Other desired amine salts may be prepared in a conventional manner by reacting the free base with a suitable organic or inorganic acid, Alternatively, a desired amine salt may be prepared directly from the trifluoroacetic salt by employing an appropriate ion exchange resin.

### Experimental Section

The synthesis of the macrocyclic intermediate XIII described below correspond to a slight modification of the published synthesis by Romesberg & al, J. Am. Chem. Soc 2007, 129 (51), 15830-38.

### Intermediate XIII: methyl(8S,11S,14S)-3,18-dimethoxy-11-methyl-14-(methylamino)-10,13-dioxo-9,12-diazatricyclo[13.3.1.1^{2,6}]icosa-1(19),2(20),3,5,15,17-hexaene-8-carboxylate

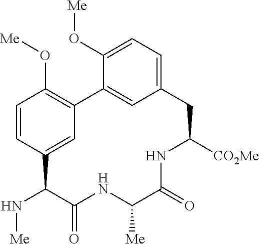 [SMILES: CN[C@@H]1C(=O)N[C@@H](C)C(=O)N[C@H](C(=O)OC)Cc2ccc(OC)c(c2)-c2cc1ccc2OC]

### Step 1: methyl(2S)-2-{[(benzyloxy)carbonyl]amino}-3-(4-hydroxyphenyl)propanoate

A 20-L 4-necked round-bottom flask were loaded with a solution of (8)-methyl 2-amino-3-(4-hydroxyphenyl)propanoate hydrochloride (1000 g, 4.27 mol, 1.00 equiv, 99%) in acetone/water (1/1; 16 L) and Na₂CO₃ (685.3 g, 6.47 mol, 1.50 equiv). To the above was added dropwise benzyl chloridocarbonate (735.3 g, 4.30 mol, 1.01 equiv) with stirring at 10-20° C. over 1 hr. The resulting mixture was stirred for 2 his at room temperature. The reaction progress was monitored by LCMS. The reaction mixture was diluted with 8 L of EtOAc, followed by extraction with 2×5 L of EtOAc. The organic layers were combined, washed with 3×1 L of 1M HCl, 2×3 L of brine, dried over anhydrous sodium sulfate, filtered and concentrated under vacuum. This resulted in 1400 g (100%) of the title compound as a white solid.

### Step 2: methyl(2S)-2-{[(benzyloxy)carbonyl]amino}-3-(4-methoxyphenyl)propanoate (II)

Into a 5-L pressure tank reactor were placed a solution of methyl(2S)-2-{[(benzyloxy)carbonyl]amino}-3-(4-hydroxyphenyl)propanoate (500 g, 1.52 mol, 1.0 equiv) in acetone (3 L), K₂CO₃ (1050 g, 7.60 mol, 5.0 equiv) and iodomethane (432.3 g, 3.07 mol, 2.04 equiv). The resulting mixture was stirred overnight at 70° C. The reaction mixture was cooled and filtered. The filtrate was diluted with 5 L of EtOAc, washed with 2×2 L of H₂O and 2 L of brine. The organic phase was dried over Na₂SO₄ and concentrated under vacuum. This resulted in 624 g (crude) of the title compound which was used without any further purification.

### Step 3: methyl(2S)-2-{[(benzyloxy)carbonyl]amino}-3-(3-iodo-4-methoxyphenyl)propanoate (III)

A 20-L 4-necked round-bottom flask purged and maintained with an inert atmosphere of nitrogen was loaded with a solution of methyl(2S)-2-{[(benzyloxy)carbonyl]amino}-3-(4-methoxyphenyl)propanoate (624 g, 1.80 mol, 1.0 equiv) in MeOH (10 L) and Ag₂SO₄ (624.4 g, 2.00 mol, 1.1 equiv). To the mixture was added I₂ (508.3 g, 2.00 mol, 1.1 equiv). The resulting mixture was stirred for 1 hr at room temperature. The reaction progress was monitored by LCMS. The reaction was quenched by the addition of 200 g of Na₂S₂O₃. The resulting mixture was concentrated under vacuum. The residue was diluted with 3 L of water, and then extracted with 2×5 L of EtOAc. The organic layers were combined, washed with 3×1 L of Na₂S₂O₃ solution and 2×3 L of brine, dried over anhydrous sodium sulfate and concentrated under vacuum. This resulted in 912.4 g (crude) of the title compound which was used without any further purification.

### Step 4: methyl(2S))-2-{[(benzyloxy)carbonyl]amino}-3-[4-methoxy-3-(4,4,5,5-tetramethyl-1,3,2-dioxaborolan-2-yl)phenyl]propanoate (IV)

A 20-L 4-necked round-bottom flask purged and maintained with an inert atmosphere of nitrogen was loaded with a solution of methyl(2S)-2-{[(benzyloxy)carbonyl]amino}-3-(3-iodo-4-methoxyphenyl)propanoate (1513 g, 3.16 mol, 1.00 equiv, 98%) in DMSO (15 L), 4,4,5,5-tetramethyl-2-(4,4,5,5-tetramethyl-1,3,2-dioxaborolan-2-yl)-1,3,2-dioxaborolane (982.5 g, 3.88 mol, 1.23 equiv), KOAc (1582.1 g, 16.14 mol, 5.11 equiv) and PdCl₂(dppf) (76.5 g, 93.75 mmol, 0.03 equiv). The resulting mixture was stirred for 2.5 hours at 80° C. in an oil bath. The reaction progress was monitored by LCMS. The reaction was quenched by the addition of 10 L of water, followed by extraction with 5×10 L of EtOAc. The organic layers were combined, washed with 2×5 L of brine, dried over anhydrous sodium sulfate and concentrated under vacuum. The residue was purified with on a silica gel column eluting with EtOAc/petroleum ether (1:10, 1:7 and 1:4). This resulted in 1200 g (77%) of the desired material as a yellow oil.

Step 5: methyl(2S)-2-amino-3-[4-methoxy-3-(4,4,5,5-tetramethyl-1,3,2-dioxaborolan-2-yl)phenyl]propanoate (V)

Into a 5-L pressure tank reactor were added a solution of methyl(2S))-2-{[(benzyloxy)carbonyl]amino}-3-[4-methoxy-3-(4,4,5,5-tetramethyl-1,3,2-dioxaborolan-2-yl)phenyl]propanoate (270 g, 518.12 mmol, 1.0 equiv, 90%) in EtOH (4 L) and Pd/C 10% (191.7 g). The resulting mixture was stirred for 4 hrs at 45° C. under a hydrogen atmosphere. The reaction mixture was cooled and filtered. The filtrate was concentrated under vacuum to give 177 g (92%) of the title compound as a brown oil which was used without any further purification.

### Step 6: (2S)-[(tert-butoxycarbonyl)amino](4-hydroxyphenyl)acetic acid (VI)

A 20-L 4-necked round-bottom flask was loaded with a solution of commercially available (S)-2-amino-2-(4-hydroxyphenyl)acetic acid (700 g, 4.19 mol, 1.0 equiv) in acetone/water (7/7 L), Na₂CO₃ (959 g, 9.05 mol, 1.5 equiv) and (Boc)₂O (667 g, 3.06 mol, 1.00 equiv). The resulting mixture was stirred overnight at room temperature. The resulting solution was adjusted to pH 3-4 with citric acid, and then extracted with 3×4 L of EtOAc. The organic layers were combined, washed with 2×3 L of brine, dried over anhydrous sodium sulfate and concentrated under vacuum. This resulted in 1000 g (89%) of the desired material as a white solid.

### Step 7: methyl(2S)-{[(2S)-2-[(-2-butoxycarbonyl)amino]-2-(4-hydroxyphenyl)acetyl]amino}propanoate (VII)

A 20-L 3-necked round-bottom flask was loaded with (2S)-[(tert-butoxycarbonyl)amino](4-hydroxyphenyl)acetic acid (750 g, 2.81 mol, 1.0 equiv), 1H-benzo[d][1,2,1]triazol-1-ol (417 g, 3.09 mol, 1.1 equiv), L-Alanine methyl ester hydrochloride (429 g, 3.09 mol, 1.10 equiv), CH₂Cl₂ (11 L) and 1-ethyl-3-(3′-dimethylaminopropyl)carbodiimide hydrochloric acid (809 g, 4.21 mol, 1.5 equiv). To the resulting solution was added dropwise TEA (738 g, 7.31 mol, 2.6 equiv) at 0° C. over 30 min. The resulting mixture was stirred overnight at room temperature. The reaction mixture was washed with 3×3 L of water, 1×2 L of 3% HCl and 2×2 L of brine. The organic phase was dried over Na₂SO₄ and concentrated under vacuum. The residue was purified on a silica gel column eluting with CH₂Cl₂/MeOH (2%). This resulted in 700 g (71%) of the desired material as a yellow solid.

### Step 8: methyl(2S)-{[(2S)-2-[(-2-butoxycarbonyl)amino]-2-(4-methoxyphenyl)acetyl]amino}propanoate (VIII)

A 20 L 4-necked round-bottom flask was loaded with a solution of methyl(2S)-{[(2S)-2-[(-2-butoxycarbonyl)amino]-2-(4-hydroxyphenyl)acetyl]amino}propanoate (780 g, 2.22 mol, 1.0 equiv) in acetone (14 L) and K₂CO₃ (1530 g, 11.09 mol, 5.0 equiv). Iodomethane (950 g, 6.69 mol, 3.4 equiv) was then added dropwise with stirring at room temperature over 0.5 hr. The resulting mixture was heated to reflux for 5 hrs. The reaction mixture was cooled and filtered. The filtrate was concentrated under vacuum. The residue was diluted with 6 L of EtOAc, washed with 3×2 L of H₂O and 2 L of brine, dried over Na₂SO₄ and concentrated under vacuum. This resulted in 350 g (43%) of the title compound as a yellow solid.

### Step 9: methyl(2S)-{[(2S)-2-[(-2-butoxycarbonyl)amino]-2-(3-iodo-4-methoxyphenyl)acetyl]amino}propanoate

The title compound was prepared as previously described in Step 3 using methyl (2S)-{[(2S)-2-[(-2-butoxycarbonyl)amino]-2-(4-methoxyphenyl)acetyl]amino}propanoate as the starting material (740 g, 2.02 mol). This resulted in 900 g (90%) of the desired material as a yellow solid.

### Step 10: (2S)-{[(2S)-2-[(-2-butoxycarbonyl)amino]-2-(3-iodo-4-methoxyphenyl)acetyl]amino}propionic acid (IX)

Into a 500-mL 4-necked round-bottom flask were placed a solution of (S)-methyl 2-((S)-2-(tert-butoxycarbonyl)-2-(3-iodo-4-methoxyphenyl)acetamido)propanoate (45 g, 91.46 mmol, 1.0 equiv) in THF (450 mL), then a solution of LiOH (2M, 90 ml, 2.0 equiv) was added. The resulting mixture was stirred for 2 hrs at room temperature, and then quenched by the addition of 60 ml of 5% NH₄Cl solution. The resulting mixture was concentrated under vacuum. The residue was diluted with 60 ml of water, the pH was adjusted to 3-4 with 20% HCl. The resulting mixture was extracted with 2×400 mL of EtOAc. The organic layers were combined, washed with 200 mL of water, 100 ml of brine, dried over anhydrous Na₂SO₄ and concentrated under vacuum to afford the desired material (40 g, 91%) as yellow oil.

### Step 11: methyl(6S,9S,12S)-6-(3-iodo-4-methoxyphenyl)-12-[4-methoxy-3-(4,4,5,5-tetramethyl-1,3,2-dioxaborolan-2-yl)benzyl]-2,2,9-trimethyl-4,7,10-trioxo-3-oxa-5,8,11 triazamidecan-13-oate (X)

A 10 L 3-necked round-bottom flask purged and maintained with an inert atmosphere of nitrogen was loaded with a solution of (2S)-{[(2S)-2-[(-2-butoxycarbonyl)amino]-2-(3-iodo-4-methoxyphenyl)acetyl]amino}propionic acid (544.8 g, 1.14 mol, 1.0 equiv) in CH₃CN/DMF (6.5 L, 2.2:1, v/v), NaHCO₃ (2.9 g, 34.52 mmol, 0.3 equiv), HOBt (436.1 g, 2.85 mol, 2.5 equiv), methyl(2S)-2-amino-3-[4-methoxy-3-(4,4,5,5-tetramethyl-1,3,2-dioxaborolan-2-yl)phenyl]propanoate (420 g, 1.13 mol, 1.0 equiv, 90%) and EDCI (480.4 g, 2.51 mol, 2.2 equiv). The resulting mixture was stirred overnight at room temperature. The pH of the reaction mixture was adjusted to 8-9 with saturated K₂CO₃ solution, then extracted with 2×5 L of EtOAc. The organic layers were combined, washed with 2×2 L of brine, dried over anhydrous Na₂SO₄ and concentrated under vacuum to afford the tilted compound (906 g, 99.8%) as a yellow oil.

### Step 12: methyl(8S,11S,14S)-14-butoxycarbonyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate (XI)

A 20-L 4-necked round-bottom flask purged and maintained with an inert atmosphere of nitrogen was loaded with a solution of methyl(6S,9S,12S)-6-(3-iodo-4-methoxyphenyl)-12-[4-methoxy-3-(4,4,5,5-tetramethyl-1,3,2-dioxaborolan-2-yl)benzyl]-2,2,9-trimethyl-4,7,10-trioxo-3-oxa-5,8,11-triazamidecan-13-oate (906 g, 1.14 mol, 1.0 equiv) in CH₃CN (15.5 L), a solution of K₃PO₄ (725.3 g, 3.42 mol, 3.00 equiv) in water (712 mL) and PdCl₂(dppf) (46.5 g, 56.94 mmol). The resulting mixture was stirred for 3 hrs at 44° C. in an oil bath, then quenched by the addition of 5 L of saturated aqueous NH₄Cl. The resulting mixture was diluted with 10 L of EtOAc. The separated water phase was extracted with 2×2 L of EtOAc. All organic layers were combined, washed with 2 L of brine, dried over anhydrous Na₂SO₄ and concentrated. The residue was purified on a silica gel column eluting with CH₂Cl₂/MeOH (80:1 then 60:1) to afford the desired material (132 g, 20%) as a brown solid.

### Step 13: methyl(8S,11S,14S)-14-amino-3,18-dimethoxy-11-methyl-10,13-dioxo-9,12-diazatricyclo[13.3.1.1^{2,6}]icosa-1(19),2(20),3,5,15,17-hexaene-8-carboxylate

Into a 2 L 3-necked round-bottom flask was placed a solution of methyl(8S,11S,14S)-14-butoxycarbonyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate (72 g, 132.8 mmol, 1.0 equiv) in CH₂Cl₂ (720 mL), then added TFA (180 mL) was added dropwise with stirring. The resulting mixture was stirred for 2 hrs at room temperature. The reaction progress was monitored by LCMS. The resulting mixture was concentrated under vacuum. The residue was diluted with 1 L of EtOAc, adjusted to pH 8 with TEA, and then concentrated under vacuum. The residue was applied onto a silica gel column and eluted with MeOH/CH₂Cl₂ (from 1:60 to 1:40 to 1:20, v/v) to afford the title compound (52.8 g, 86%) as a light red solid.

### Step 14: methyl(8S,11S,14S)-3,18-dimethoxy-11-methyl-14-{[(4-nitrophenyl)sulfonyl]-10,13-dioxo-9,12-diazatricyclo[13.3.1.1^{2,6}]icosa-1(19),2(20),3,5,15,17-hexaene-8-carboxylate (XII)

To a solution of methyl(8S,11S,14S)-14-amino-3,18-dimethoxy-11-methyl-10,13-dioxo-9,12-diazatricyclo[13.3.1.1^{2,6}]icosa-1(19),2(20),3,5,15,17-hexaene-8-carboxylate (22.6 g, 51.2 mmol, 1.0 equiv, 99%) in CH₃CN (500 mL) was added TEA (17.1 mL, 123 mmol, 2.4 equiv), followed by portionwise addition of 4-nitrobenzene-1-sulfonyl chloride (13.61 g, 61.4 mmol, 1.2 equiv). The mixture was stirred for 3 hrs at room temperature. The resulting precipitate was collected by filtration and wash with CH₃CN (50 mL) to the desired material (31.5 g, 98%) as a yellow solid.

### Step 15: methyl(8S,11S,14S)-3,18-dimethoxy-11-methyl-14-{methyl[(4-nitrophenyl)sulfonyl]-10,13-dioxo-9,12-diazatricyclo[13.3.1.1^{2,6}]icosa-1(19),2(20),3,5,15,17-hexaene-8-carboxylate

To suspension of methyl(8S,11S,14S)-3,18-dimethoxy-11-methyl-14-{[(4-nitrophenyl)sulfonyl]-10,13-dioxo-9,12-diazatricyclo[13.3.1.1^{2,6}]icosa-1(19),2(20),3,5,15,17-hexaene-8-carboxylate (31.5 g, 50.3 mmol, 1.0 equiv) in acetone (0.5 L) was added K₂CO₃ (35.0, 253 mmol, 5.0 equiv) and iodomethane (16 mL, 256 mmol, 5 equiv). The resulting mixture was stirred overnight at room temperature. The reaction was monitored by LCMS. The resulting mixture was filtered and the supernatant concentrated. The residue was partially dissolved in CH₂Cl₂ (300 mL) and purified on a pad of silica gel (500 g) eluting with 2 L of CH₂Cl₂ then 4 L of 5% MeOH/CH₂Cl₂ to afford the desired material (25.3 g, 79%) as a yellow foam.

### Step 16: methyl(8S,11S,14S)-3,18-dimethoxy-11-methyl-14-(methylamino)-10,13-dioxo-9,12-diazatricyclo[13.3.1.1^{2,6}]icosa-1(19),2(20),3,5,15,17-hexaene-8-carboxylate (XIII)

To a solution of methyl(8S,11S,14S)-3,18-dimethoxy-11-methyl-14-{methyl[(4-nitrophenyl)sulfonyl]-10,13-dioxo-9,12-diazatricyclo[13.3.1.1^{2,6}]icosa-1(19),2(20),3,5,15,17-hexaene-8-carboxylate (25.3 mL, 39.5 mmol, 1.0 equiv) in acetonitrile (500 mL) was added 2-mercaptoacetic acid (18 mL, 259 mmol, 6.6 equiv, 99%) and 2,3,4,6,7,8,9,10-octahydropyrimido[1,2-a]azepine (60 mL, 398 mmol, 10.1 equiv). The resulting mixture was stirred for 4 hr at room temperature and was concentrated. The resulting residue was diluted in EtOAc (1 L), washed with saturated aqueous NaHCO₃ (2×500 mL), water (2×500 mL), brine (500 mL), dried over anhydrous Na₂SO₄, filtered and concentrated. The residual solid was dissolved in 200 mL of hot EtOAc and triturated slowly with 200 mL of Hex. The resulting solid was filtered to afford the tilted compound (15.2 g, 85%) as a white solid. LCMS (+ESI) m/z 456.4

### Example 1

### Method B

### 6-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium trifluoroacetate

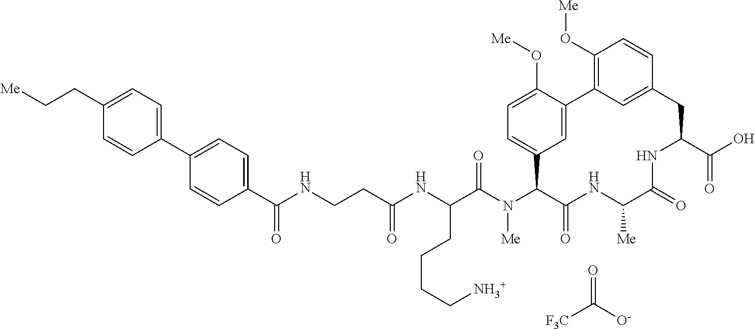 [SMILES: CCCc1ccc(-c2ccc(C(=O)NCCC(=O)NC(CCCC[NH3+])C(=O)N(C)[C@@H]3C(=O)N[C@@H](C)C(=O)N[C@H](C(=O)O)Cc4ccc(OC)c(c4)-c4cc3ccc4OC)cc2)cc1 ; O=C([O-])C(F)(F)F]

### Step 1: methyl(2S)-2-[(3-{[(benzyloxy)carbonyl]amino}propanoyl)amino]-6-[(tert-butoxycarbonyl)amino]hexanoate

To a solution of methyl(2S)-2-amino-6-[(tert)-butoxycarbonyl)amino]hexanoate hydrochloride (7 g, 23.6 mmol) in 40 mL of DMF were added HATU (10.76 g, 28.3 mmol), 3-{[(benzyloxy)carbonyl]amino}propanoic acid (6.32 g, 28.3 mmol) and then DIPEA (12.36 ml, 70.8 mmol). The resulting solution was stirred for 18 hrs and then partitioned between saturated aqueous solution NaHCO₃ and EtOAc. The two phases were separated and the aqueous layer was extracted twice with EtOAc. The combined organic phases were washed with water (2×) and brine, dried over Na₂SO₄ and concentrated under reduced pressure. Purification by ISCO (220 g cartridge of silica gel) eluting with 30%-90% EtOAc in Hex afforded the desired material (3.33 g, 30%) as a white solid.

### Step 2: methyl(2S)-2-[(3-aminopropanoyl)amino]-6-[(tert-butoxycarbonyl)amino]hexanoate

To a solution of methyl(2S)-2-[(3-{[(benzyloxy)carbonyl]amino}propanoyl)amino]-6-[(tert-butoxycarbonyl)amino]hexanoate (3.30 g, 7.09 mmol) in ethanol (40 mL) was added Pd/C 10% (0.754 g, 7.09 mmol). The resulting suspension was stirred over 1 atmosphere of hydrogen at room temperature for 5 hours. The catalyst was filtered over celite and the solvent was removed in vacuo to afford the title compound (2.35 g, 100%) as a colorless oil.

### Step 3: methyl(2S)-6-[(tert-butoxycarbonyl)amino]-2-[(3-{[(4′-propylbiphenyl-4-yl)carbonyl]amino}propanoyl)amino]hexanoate

To a solution of methyl(2S)-2-[(3-aminopropanoyl)amino]-6-[(tert-butoxycarbonyl)amino]hexanoate (2.349 g, 7.09 mmol) in DMF (15 mL) were added HOBT (1.628 g, 10.63 mmol), 4-(4-N-propylphenyl)benzoic acid (2.044 g, 8.51 mmol) and EDCI (2.038 g, 10.63 mmol). The solution was stirred at room temperature for 5 hrs and then partitioned between water and EtOAc. The product was extracted with EtOAc and the combined organic phases were washed with a saturated aqueous NaHCO₃, water and brine, dried over sodium sulfate and filtered. The solvent was removed under reduced pressure and the crude residue was purified by ISCO (silica gel, 120 g cartridge) eluting with 30%-100% EtOAc in Hex and then it was stirred in EtOAc 15% MeOH to give after filtration the desired material (1.91 g, 49%) as a white waxy solid.

### Step 4: (2S)-6-[(tert-butoxycarbonyl)amino]-2-[(3-{[(4′-propylbiphenyl-4-yl)carbonyl]amino}propanoyl)amino]hexanoic acid

To a suspension of methyl(2S)-6-[(tert-butoxycarbonyl)amino]-2-[(3-{[(4′-propylbiphenyl-4-yl)carbonyl]amino}propanoyl)amino]hexanoate (1.91 g, 3.45 mmol) in 40 mL THF/MeOH (1/1) was added 2M LiOH (10 ml, 20.0 mmol). The resulting mixture was stirred at 40° C. for 4 hours and the organic solvents were removed in vacuo. The aqueous layer was poured into a saturated aqueous NH₄Cl and 1N HCl was added slowly to adjust the pH to 4. The product was extracted with hot EtOAc (2×) and the combined organic phases were washed with water and brine, dried over sodium sulfate and concentrate under reduced pressure to afford the title compound (1.72 g, 93%) as a white solid.

### Step 5: methyl(8S,11S,14S)-14-[{6-[(tert-butoxycarbonyl)amino]-2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate

To a solution of methyl(8S,11S,14S)-3,18-dimethoxy-11-methyl-14-(methylamino)-10,13-dioxo-9,12-diazatricyclo[13.3.1.1^{2,6}]icosa-1(19),2(20),3,5,15,17-hexaene-8-carboxylate (XIII, 1.23 g, 2.69 mmol) in DMF were added HOST (1.24 g, 8.07 mmol), (2S)-6-[(tert-butoxycarbonyl)amino]-2-[(3-{[(4′-propylbiphenyl-4-yl)carbonyl]amino}propanoyl)amino]hexanoic acid (1.72 g, 3.19 mmol), DIPEA (1.41 nil, 8.07 mmol) and the coupling reagent EDCI (1.55 g, 8.07 mmol). The resulting solution was stirred at room temperature for 18 hrs and then water was added until the compound precipitated. The product was filtered and it was triturated in water for 15 min to get an off-white solid after filtration. The product was purified by ISCO (silica gel, 80 g cartridge) eluting with EtOAc/MeOH (0% to 20%) in to give the desired compound (1.73 g, 66%) a white solid. Upon careful examination of the NMR of the title compound, it was found that the lysine residue had epimerized. The resulting diastereoisomers were inseparable by normal or reverse phase chromatography. Racemization was found to occur during the coupling reaction described in Step 5.

### Step 6: (8S,11S,14S)-14-[{6-[(tert-butoxycarbonyl)amino]-2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylic acid

To a solution of methyl(8S,11S,14S)-14-[{6-[(tert-butoxycarbonyl)amino]-2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate (1.20 g, 1.23 mmol) in 20 mL THF/MeOH (1/1) was added 2M LiOH (4.5 ml, 9.00 mmol). The resulting mixture was stirred at room temperature for 1 hour and the organic solvents were removed in vacuo. Saturated aqueous NH₄Cl was added to the remaining aqueous layer and pH was adjusted to 4 using aqueous HCl (1N). The product was first extracted with THF and then twice with a mixture of EtOAc/THF (2/1). The combined organic extracts were washed with water and brine, dried over sodium sulfate and concentrate under reduced pressure. The product was triturated in a mixture of EtOAc/Hex to afford the tilted compound (1.11 g, 94%) as a white solid.

### Step 7: EXAMPLE 1

To a suspension of (8S,11S,14S)-14-[{6-[(tert-butoxycarbonyl)amino]-2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylic acid (1.099 g, 1.14 mmol) in 15 mL of CH₂Cl₂ (15 ml) was added TFA (5.0 ml, 65 mmol). After 1 hr of stirring at room temperature, the reaction mixture was evaporated in vacuo and the crude residue was dissolved in CH₃CN/water and it was lyophilized to get the desired material (1.1 g, 100%) as a white solid. LRMS (ESI): (calc) 862.43 (found) 863.50 (MH⁺).

### Example 2

### Method B

### 5-(R,S)-6-[[(7S,10S,3S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium trifluoroacetate

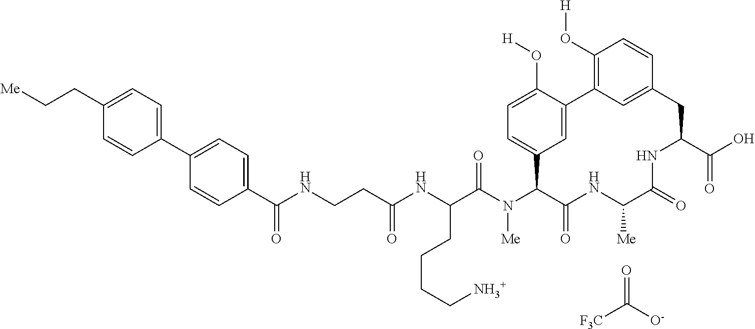 [SMILES: CCCc1ccc(-c2ccc(C(=O)NCCC(=O)NC(CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F]

To a suspension of 6-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium trifluoroacetate (EXAMPLE 1) (1.10 g, 1.136 mmol) in 20 mL of 1-propanethiol was added portionwise AlBr₃ (4.70 g, 17.60 mmol). The resulting mixture was stirred at 50° C. for 1 hr and was then cooled to room temperature. Water was carefully added to quench the reaction and the white solid was filtered. The filtered cake was washed with water and it was dried by aspiration. The crude solid was purified by flash column chromatography on Lichoprep RP-18 eluting with CH₃CN (0% to 75%) in water. Fractions containing the desired material were combined. TFA (0.5 mL) was added to the combined fractions. They were concentrated under vacuum until most of the CH₃CN was evaporated and prior to precipitation. The resulting aqueous phase was lyophilized to afford the title compound (0.67, 62%) as a white solid. LRMS (ESI): (calc) 834.40 (found) 835.45 (MH⁺)

### Example 3

### Method C

### (5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium trifluoroacetate

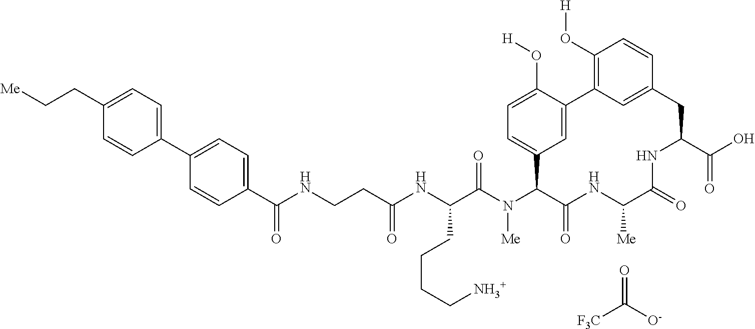 [SMILES: CCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F]

The procedures described below (Method C) for the synthesis of EXAMPLE 3 avoids the epimerization of the lysine residue.

### Step 1: methyl 3-{[(4′-propylbiphenyl-4-yl)carbonyl]amino}propanoate

To a solution of amine β-Alanine methyl ester hydrochloride (2.67 g, 19.1 mmol) in DMF (30 mL) were added HATU (7.28 g, 19.1 mmol), 4-(4-n-propylphenyl)benzoic acid (2.30 g, 9.6 mmol) and DIPEA (6.69 ml, 38.3 mmol). The solution was stirred at room temperature for 4 days. The reaction mixture was diluted with water and the resulting precipitate was filtered. The filtered cake was washed with water and dried under vacuum to afford the desired material (3.11 g, 100%).

### Step 2: 3-{[(4′-propylbiphenyl-4-yl)carbonyl]amino}propionic acid

To a solution of methyl 3-{[(4′-propylbiphenyl-4-yl)carbonyl]amino}propanoate (3.11 g, 9.56 mmol) in 100 mL of THF/MeOH 1:1 was added 2M LiOH (20 ml, 40.0 mmol). The resulting mixture was stirred at 50° C. for 3 hours and the organic solvents were removed in vacuo to one third volume. The resulting aqueous layer was acidified with 6N HCl to pH 2 and the resulting precipitate was filtered. The filtered cake was washed with water and dried by aspiration. The product was suspended in CH₃CN and then solvent was removed under reduced pressure to remove the remaining water to afford the tilted compound (2.84 g, 95%) as an off-white solid.

### Step 3: methyl(8S,11S,14S)-14-[{(2S)-2-{[(benzyloxy)carbonyl]amino}-6-[(tert-butoxycarbonyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate

To a solution of methyl(8S,11S,14S)-3,18-dimethoxy-11-methyl-14-(methylamino)-10,13-dioxo-9,12-diazatricyclo[13.3.1.1^{2,6}]icosa-1(19),2(20),3,5,15,17-hexaene-8-carboxylate (2.06 g, 4.52 mmol) in DMF were added HOBT (2.08 g, 13.6 mmol), DIPEA (2.37 ml, 13.6 mmol), (2S)-2-{[(benzyloxy)carbonyl]amino}-6-[(tert-butoxycarbonyl)amino]hexanoic acid (1.89 g, 4.97 mmol) and the coupling reagent EDCI (2.60 g, 13.6 mmol). The resulting solution was stirred at room temperature for 20 hrs and then was diluted with water. The precipitate was filtered and the resulting cake was washed with water and dried by aspiration. The product was purified by ISCO (silica gel, 80 g cartridge) eluting with CH₂Cl₂/MeOH (0 to 10%) to yield the desired material (2.67 g, 72%) as a white solid after trituration in Hex.

### Step 4: methyl(8S,11S,14S)-14-[{(2S)-2-amino-6-[(tert-butoxycarbonyl)amino]hexanoyl}(methyl)amino]-3,18-dimethoxy-11-methyl-10,13-dioxo-9,12 diazatricyclo[13.3.1.1^{2,6}]icosa-1(19),2(20),3,5,15,17-hexaene-8-carboxylate

To a suspension of methyl(8S,11S,14S)-14-[{(2S)-2-{[(benzyloxy)carbonyl]amino}-6-[(tert-butoxycarbonyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate (1.79 g, 2.188 mmol) and 10% Pd/C (1 g, 0.94 mmol) in 25 mL of ethanol was added 1,4-cyclohexadiene (8 ml, 85 mmol). After 48 hours of stirring, the catalyst was filtered and the solvent was evaporated to afford the desired material (1.40 g, 93%) which was used without further purification.

### Step 5: methyl(8S,11S,14S)-14-[{(2S)-6-[(tert-butoxycarbonyl)amino]-2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate

To a solution of methyl(8S,11S,14S)-14-[{(2S)-2-amino-6-[(tert-butoxycarbonyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate (1.40 g, 2.044 mmol) in DMF were added HOBT (0.94 g, 6.13 mmol), DIPEA (1.07 ml, 6.13 mmol), 3-{[(4′-propylbiphenyl-4-yl)carbonyl]amino}propionic acid from Step 2 (0.70 g, 2.25 mmol) and the coupling reagent EDCI (1.18 g, 6.13 mmol). The solution was stirred at room temperature for 18 hrs and was diluted with water. The precipitate was filtered and the resulting cake was washed with water and dried by aspiration. The product was purified by ISCO (silica gel, 80 g cartridge) eluting with EtOAc (50 to 100%) in Hex followed by MeOH (0 to 20%) in EtOAc to afford the title compounds (1.52 g, 76%) as a white solid after trituration in Hex.

### Step 6: (5S)-6-[[(7S,10S,13S)-3,18-dimethoxy-13-(methoxycarbonyl)-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium trifluoroacetate

To a solution of methyl(8S,11S,14S)-14-[{(2S)-6-[(tert-butoxycarbonyl)amino]2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate (1.52 g, 1.56 mmol) in 15 mL of CH₂Cl₂ was added TFA (8 ml, 1.56 mmol). After 45 min of stirring at room temperature, toluene was added and the solution was concentrated under reduced pressure to dryness to afford the desired material (1.54 g, 100%) which was used as such in the next step.

### Step 7: EXAMPLE 3

To a solution of (5S)-6-[[(7S,10S,13S)-3,18-dimethoxy-13-(methoxycarbonyl)-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium trifluoroacetate (1.54 g, 1.56 mmol) in 1-propanethiol (20 ml) was added one portion of 2.18 g of AlBr₃. After 2 hours of stirring at 50° C. the reaction, 1.50 g of additional AlBr₃ was added. The sequence was repeated one more time by adding 0.558 g of AlBr₃. After 2 more hrs at 50° C., the reaction was quenched by adding 3 mL of water dropwise and an excess of methanol. The mixture was concentrated under reduced pressure to dryness and the crude residue was purified by ISCO(C18, 130 g cartridge) eluting with water (1% TFA)/MeOH (15 to 80%). The pure fractions were combined concentrated and the residual aqueous solution was lyophilized to afford a white fluffy solid (793 mg, 54%). LRMS (ESI): (calc) 834.40 (found) 835.45 (MH⁺).

### Example 4

### Method C

### (5R)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbipherryl-4-yl)carbonyl]amino}propanoyl)amino]hexan-1-aminium trifluoroacetate

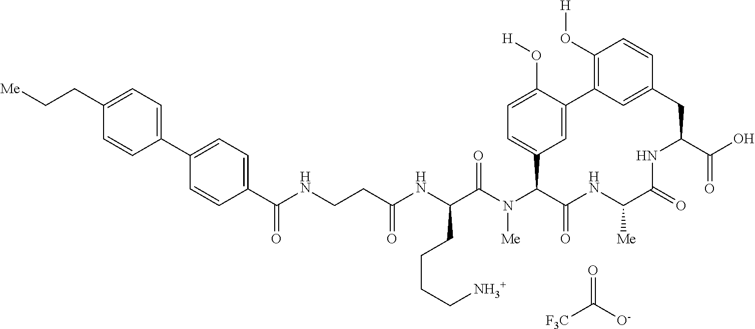 [SMILES: CCCc1ccc(-c2ccc(C(=O)NCCC(=O)N[C@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F]

EXAMPLE 4 was prepared according to the procedure described for EXAMPLE 3 but using (2R)-2-{[(benzyloxy)carbonyl]amino}-6-[(tert-butoxycarbonyl)amino]hexanoic acid in Step 3. LRMS (ESI): (calc) 834.40 (found) 835.45 (MH⁺).

### Example 5

### Method 5

### (5R,S)-6-[[(7S,10S,13S)-3,18-dihydroxy-13-(hydroxymethyl)-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium trifluoroacetate

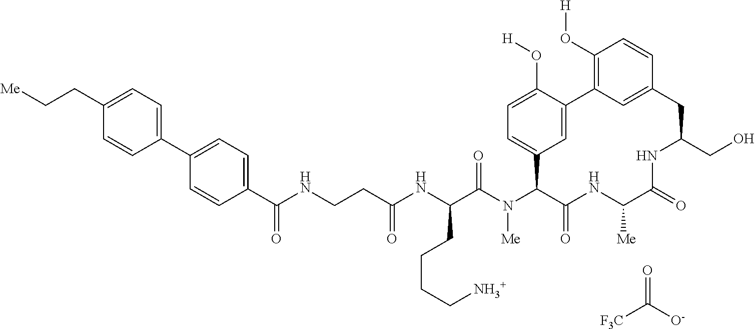 [SMILES: CCCc1ccc(-c2ccc(C(=O)NCCC(=O)N[C@H](CCCC[NH3+])C(=O)N(C)[C@@H]3C(=O)N[C@@H](C)C(=O)N[C@H](CO)Cc4ccc(O)c(c4)-c4cc3ccc4O)cc2)cc1 ; O=C([O-])C(F)(F)F]

### Step 1: tert-butyl[(5R,S)-6-[[(7S,10S,13S)-13-(hydroxymethyl)-3,18-dimethoxy-10-methyl-8,11-dioxo-9,12-diazatricyclo[13.3.1.12,6]icosa-1(19),2(20),3,5,15,17-hexaen-7yl]-(methyl)amino]-6-oxo-5-[(3-{[(4′-propylbiphenyl-4-yl)carbonyl]amino}propanoyl)amino]hexyl}carbamate

To a suspension of methyl(8S,11S,14S)-14-[{6-[(tert-butoxycarbonyl)amino]-2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate from Step 5 of EXAMPLE 1 (273 mg, 0.279 mmol) in 6 mL of THF was added LiBH₄ (60.9 mg, 2.79 mmol). The resulting mixture was stirring 90 minutes at 50° C., the solution was cooled at room temperature and it was quenched by the addition of saturated aqueous NH₄Cl. The product was extracted with EtOAc (2×) and the combined organic extracts were washed with water and brine, dried over Na₂SO₄ and concentrated. The residue was purified by ISCO (silica gel, 4 g cartridge) eluting with CH₂Cl₂/MeOH (0 to 15%) to afford the title compound (188 mg 71%) as a white solid. LRMS (ESI): (calc) 948.50 (found) 950.5 (MH⁺)

### Step 2: EXAMPLE 5

To a suspension of tert-butyl[(5R,S)-6-[[(7S,10S,13S)-13-(hydroxymethyl)-3,18-dimethoxy-10-methyl-8,11-dioxo-9,12-diazatricyclo[13.3.1.12,6]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexyl}carbamate (80 mg, 0.084 mmol) in 3 mL of CH₂Cl₂ and 1.5 mL of propanethiol was added AlBr₃ (350 mg, 1.31 mmol). The mixture was stirred for 2 hrs at 50° C., cooled at room temperature, quench with water (1 mL) and diluted with methanol (20 mL). The solution was concentrated under reduced pressure and the crude residue was purified by ISCO (C18, 15.5 g cartridge) eluting with water (1% TFA) MeOH (0 to 90%). The pure fractions were combined, concentrated and the residual aqueous solution was lyophilized to afford the title compound (45 mg, 57%) as a white solid. LRMS (ESI): (calc) 820.42 (found) 821.3 (WO.

### Example 6

### Method B

### 6-[[(7S,10S,13S)-13-(aminocarboxyl)-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium trifluoroacetate

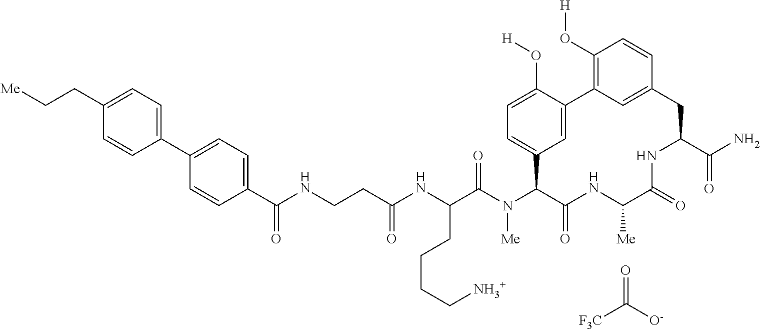 [SMILES: CCCc1ccc(-c2ccc(C(=O)NCCC(=O)NC(CCCC[NH3+])C(=O)N(C)[C@@H]3C(=O)N[C@@H](C)C(=O)N[C@H](C(N)=O)Cc4ccc(O)c(c4)-c4cc3ccc4O)cc2)cc1 ; O=C([O-])C(F)(F)F]

### Step 1: tert-butyl{6-[[(7S,10S,15S)-13-(aminocarbonyl)-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexyl}carbamate

To a solution of (8S,11S,14S)-14-[{6-[(tert-butoxycarbonyl)amino]-2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylic acid (37 mg, 0.038 mmol) in DMF (1.5 mL) was added HATU (33.8 mg, 0.089 mmol), NH₄C1 (20.1 mg, 0.376 mmol) and DIPEA (81 μL, 0.46 mmol). The resulting solution was stirred overnight at room temperature. The reaction mixture was diluted with CH₂Cl₂, wash with 1N aqueous NaOH, dried over MgSO₄, filtered and concentrated to afford the desired material (22 mg, 60%) as a white solid which was used without any further purification.

### Step 2: (8S,11S,14S)-14-[{6-amino-2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxamide

To a solution of tert-butyl{6-[[(7S,10S,13S)-13-(aminocarbonyl)-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexyl}carbamate (22 mg, 0.023 mmol) in CH₂Cl₂ (3 mL) was added TEA (1.0 mL). The mixture was stirred at room temperature for 1.5 hr, concentrated and co-evaporated with CH₂Cl₂ (3×1 mL) to afford the desired material (19.7 mg) which was use as such in the next step.

### Step 3: EXAMPLE 6

To a suspension of (8S,11S,14S)-14-[{6-amino-2-[(3-{[(4′-propylbiphenyl-4-yl)carbonyl]amino}propanoyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxamide (19.7 mg, 0.023 mmol) in 2 mL of 1-propanethiol was added dropwisewise AlBr₃ (0.34 mL, 1M in CH₂Br₂, 0.34 mmol). The resulting mixture was stirred at 50° C. for 4 hr and was then cooled to room temperature. Water was carefully added to quench the reaction and the resulting mixture was concentrated. The residue was dissolved in MeOH and purified by HPLC (MassLynx) eluting with increment amount of CH₃CN in water (0.1% TFA). Fractions containing the desired material were combined, concentrated and the resulting aqueous phase was lyophilized to afford the title compound (12.7, 67%) as a white solid. LRMS (ESI): (calc) 833.41 (found) 834.67 (MH⁺)

### Example 7

### Method B

### 6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-({3-[(1,1′:4′,1″-terphenyl-4-ylcarbonyl)amino]propanoyl}amino)hexan-1-aminium trifluoroacetate

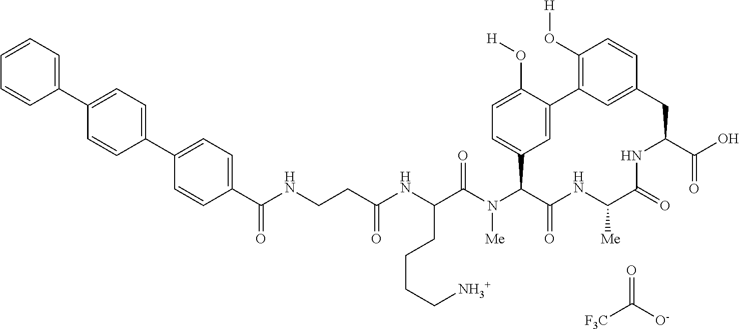 [SMILES: C[C@@H]1NC(=O)[C@@H](N(C)C(=O)C(CCCC[NH3+])NC(=O)CCNC(=O)c2ccc(-c3ccc(-c4ccccc4)cc3)cc2)c2ccc(O)c(c2)-c2cc(ccc2O)C[C@@H](C(=O)O)NC1=O ; O=C([O-])C(F)(F)F]

EXAMPLE 7 can be prepared according to the procedure described for EXAMPLE 1 but using commercially available 1,1′:4′,1″-terphenyl-4-carboxylic acid in Step 3. LRMS (ESI): (calc) 868.38 (found) 869.79 (MH⁺)

### Example 8

### Method C

### ({(4S)-5-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-oxo-4-[(3-{[(4′-propylbiphenyl-4-yl)carbonyl]amino}propanoyl)amino]pentyl}amino)(imino)methanaminium trifluoroacetate

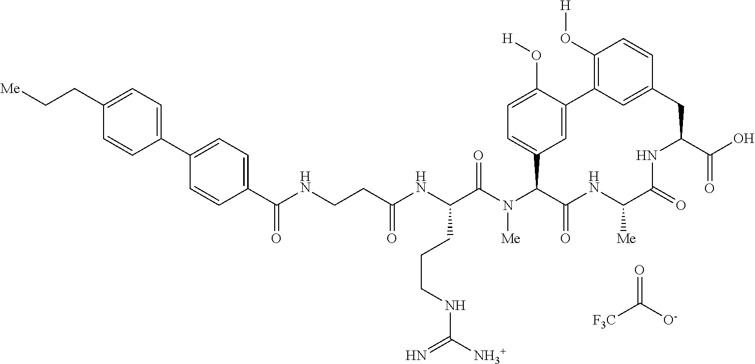 [SMILES: CCCc1ccc(-c2ccc(C(=O)NCCC(=O)N[C@@H](CCCNC(=N)[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F]

EXAMPLE 8 was prepared according to the procedure described for EXAMPLE 3 but using commercially available tert-butyl((1S)-1-formyl-4-{[imino(nitroamino)methyl]amino}butyl)carbamate in Step 3. An additional step was required to remove the guanidine nitro protecting group prior to the final AlBr₃/ethanethiol final deprotection. The reaction was performed as follow. To a solution of methyl(8S,11S,14S)-14-[{(2S)-5-{[imino(nitroamino)methyl]amino}-2-[(3-{[(4′-propylbiphenyl-4-yl)carbonyl]amino}propanoyl)amino]pentanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate (28 mg, 0.030 mmol) in EtOH (95%, 1.5 mL) and acetic acid (0.5 mL) was added pd/c 10% (10 mg). The mixture was stirred over an atmosphere of H₂ for 12 hr. The reaction was monitored by LCMS. The resulting mixture was filtered on Celite and concentrated to afford the desired methyl(8S,11S,14S)-14-[{(2S)-5-{[imino(amino)methyl]amino}-2-[(3-{[(4′-propylbiphenyl-4-yl)carbonyl]amino}propanoyl)amino]pentanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate (27 mg, 100%) which was used without any further purification. LRMS (ESI): (calc) 862.40 (found) 863.79 (MH⁺).

### Example 9

### Method B

### 6-[[(7S,10S,13S)-13-carboxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium trifluoroacetate

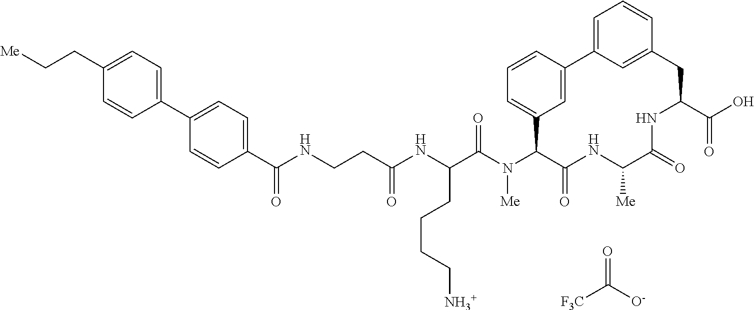 [SMILES: CCCc1ccc(-c2ccc(C(=O)NCCC(=O)NC(CCCC[NH3+])C(=O)N(C)[C@@H]3C(=O)N[C@@H](C)C(=O)N[C@H](C(=O)O)Cc4cccc(c4)-c4cccc3c4)cc2)cc1 ; O=C([O-])C(F)(F)F]

### Step 1: (8S,11S,14S)-14-amino-3,18-dihydroxy-11-methyl-10,13-dioxo-9,12-diazatricyclo[13.3.1.1^{2,6}]icosa-1(19),2(20),3,5,15,17-hexaene-8-carboxylic acid

To a suspension of methyl(8S,11S,14S)-14-amino-3,18-dimethoxy-11-methyl-10,13-dioxo-9,12-diazatricyclo[13.3.1.1^{2,6}]icosa-1(19),2(20),3,5,15,17-hexaene-8-carboxylate (Step 13, XIII) (3.0 g, 5.40 mmol) in propanethiol (12 mL) was added portion wise AlBr₃ (14.4 g, 54.0 mmol) at 0° C. over 1 hr. The mixture was stirred at room temperature for 5 hrs, cooled to 0° C. and quenched with 30 mL of water. The volatiles were evaporated in vacuo and the residue was dissolved in water. Purification by reverse phase flash chromatography using LiChroPrep RP-18 and eluting with water then 1 to 2% CH₃CN afforded the desired material (2.10 g, 97%), after clean fractions were combined and lyophilized, as a white solid. Two purifications were necessary to obtain clean material.

### Step 2: methyl(8S,11S,14S)-14-amino-3,18-dihydroxy-11-methyl-10,13-dioxo-9,12-diazatricyclo[13.3.1.1^{2,6}]icosa-1(19),2(20),3,5,15,17-hexaene-8-carboxylate

To a solution of (8S,11S,14S)-14-amino-3,18-dihydroxy-11-methyl-10,13-dioxo-9,12-diazatricyclo[13.3.1.1^{2,6}]icosa-1(19),2(20),3,5,15,17-hexaene-8-carboxylic acid (800 mg, 2.00 mmol) in MeOH (10 mL) was added acetyl chloride (142 pt, 2.00 mmol). The mixture was refluxed for 18 hrs until no starting material was observed by reverse phase TLC (CH₃CN/H₂O, 1/9). The reaction mixture was cooled to room temperature, quenched with saturated aqueous NH₄OH, and concentrated in vacuo. The residue was purified by flash chromatography on silica gel eluting with CH₂Cl₂/MeOH/NH₄OH (80/18/2) to afford the title compound (760 mg, 92%) as a white solid.

### Step 3: methyl(8S,11S,14S)-14-[(tert-butoxycarbonyl)amino]-3,18-dihydroxy-11-methyl-10,13-dioxo-9,12-diazatricyclo[13.3.1.1^{2,6}]icosa-1(19),2(20),3,5,15,17-hexaene-8-carboxylate

To a solution of methyl(8S,11S,14S)-14-amino-3,18-dihydroxy-11-methyl-10,13-dioxo-9,12-diazatricyclo[13.3.1.1^{2,6}]icosa-1(19),2(20),3,5,15,17-hexaene-8-carboxylate (760 mg, 1.84 mmol) in DMF (18 mL) was added NEt₃ (0.31 mL, 2.21 mmol) and BOC-anhydride (441 mg, 2.02 mmol). The mixture was stirred overnight at room temperature. The resulting mixture was diluted with EtOAc, washed with brine, dried over Na₂SO₄, filtered and concentrated. The residue was purified by flash chromatography on silica gel eluting with EtOAc/Hex (7/3) then EtOAc/MeOH (99/1) to afford the desired compound (850 mg, 90%) as a white solid.

### Step 4: methyl(8S,11S,14S)-14-[(tert-butoxycarbonyl)amino]-3,18-bis{[(trifluoromethyl)sulfonyl]oxy}-11-methyl-10,13-dioxo-9,12-diazatricyclo[13.3.1.1^{2,6}]icosa-1(19),2(20),3,5,15,17-hexaene-8-carboxylate

To a solution of methyl(8S,11S,14S)-14-[(tert-butoxycarbonyl)amino]-3,18-dihydroxy-11-methyl-10,13-dioxo-9,12-diazatricyclo[13.3.1.1^{2,6}]icosa-1(19),2(20),3,5,15,17-hexaene-8-carboxylate (850 mg, 1.66 mmol) in CH₂Cl₂ (6 mL) was added NEt₃ (2.3 mL, 16.6 mmol). The mixture was cooled to 0° C. before trifluoromethane sulfonic anhydride (0.70 mL, 4.1 mmol) was added dropwise. The resulting mixture was warm to room temperature and an extra amount of Tf₂O (0.5 mL) was added to complete the reaction. The final mixture was stirred for 30 min then was poured in saturated aqueous NaHCO₃. and extracted with EtOAc. The organic extract was washed with brine, dried over Na₂SO₄, filtered and concentrated. Flash chromatography on silica gel eluting with EtOAc/Hex (1/1) afforded the desired compound (750 mg, 58%) as a yellow solid.

### Step 5: methyl(8S,11S,14S)-14-[(tert-butoxycarbonyl)amino]-11-methyl-10,13-dioxo-9,12-diazatricyclo[13.3.1.1^{2,6}]icosa-1(19),2(20),3,5,15,17-hexaene-8-carboxylate

To a mixture of methyl(8S,11S,14S)-14-[(tert-butoxycarbonyl)amino]-3,18-bis{[(trifluoromethyl)sulfonyl]oxy}-11-methyl-10,13-dioxo-9,12-diazatricyclo[13.3.1.1^{2,6}]icosa-1(19),2(20),3,5,15,17-hexaene-8-carboxylate (750 mg, 0.96 mmol) and PdCl₂(dppf)₂ (158 mg, 0.19 mmol) in DMF (13.5 mL) was added HCOOH (0.15 mL, 3.9 mmol) and NEt₃ (0.81 mL, 5.8 mmol). The final mixture was degassed twice (high vacuum then filled with nitrogen) and stirred at 85° C. for 2.5 hrs. The resulting reaction mixture was cooled to room temperature, diluted with EtOAc, washed twice with water, brine, dried over Na₂SO₄, filtered and concentrated. The residue was dissolved in hot THF/MeOH/EtOAc add submitted to flash chromatography, eluting with Tol/EtOAc (1/1) to afford the desired material (400 mg, 86%) as a yellow solid.

### Step 6: methyl(8S,11S,14S)-14-(methylamino)-10,13-dioxo-9,12-diazatricyclo[13.3.1.1^{2,6}]icosa-1(19),2(20),3,5,15,17-hexaene-8-carboxylate

The titled compound was prepared according to the procedure described for the Intermediate XIII, Step 13 to 16.

EXAMPLE 9 was prepared according to the procedures described for EXAMPLE 3 but using methyl(8S,11S,14S)-14-(methylamino)-10,13-dioxo-9,12-diazatricyclo[13.3.1.1^{2,6}]icosa-1(19),2(20),3,5,15,17-hexaene-8-carboxylate in Step 3. LRMS (ESI): (calc) 802.41 (found) 804.4 (MH⁺)

### Example 10

### Method B

### 5-{[3-({4-[(4-butylphenyl)ethynyl]benzoyl}amino)propanoyl]amino}-6-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxohexart-1-aminium trifluoroacetate

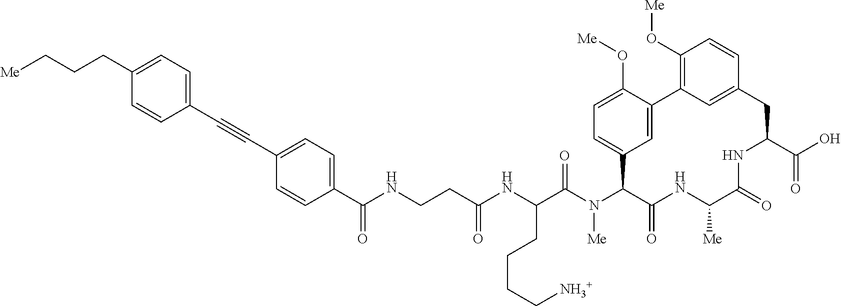 [SMILES: CCCCc1ccc(C#Cc2ccc(C(=O)NCCC(=O)NC(CCCC[NH3+])C(=O)N(C)[C@@H]3C(=O)N[C@@H](C)C(=O)N[C@H](C(=O)O)Cc4ccc(OC)c(c4)-c4cc3ccc4OC)cc2)cc1]

### Step 1: methyl 4-[(4-butylphenyl)ethynyl]benzoate

A mixture of 1-butyl-4-ethynylbenzene (1.21 g, 7.63 mmol), methyl 4-iodo benzoate (1.0 g, 3.82 mmol), CuI (145 mg, 0.76 mmol), Pd(PPh₃)₄ (220 mg, 0.19 mmol) and NEt₃ (3.2 mL, 22.9 mmol) in DMF (10 mL) was stirred at 65° C. overnight. The resulting mixture was cooled to room temperature, diluted with EtOAc, washed with water, brine, dried over Na₂SO₄, filtered and concentrated. The residue was purified by ISCO (silica gel, 80 g cartridge) eluting with Hex/EtOAc (0 to 20%) to afford the tilted compound (1.0 g, 90%).

### Step 2: 4-[(4-butylphenyl)ethynyl]benzoic acid sodium salt

To a solution of methyl 4-[(4-butylphenyl)ethynyl]benzoate (1.0 g, 3.42 mmol) in THF (16 mL) and MeOH (8 mL) was added 2N aqueous NaOH (1.71 mL, 3.42 mmol). The mixture was stirred at room temperature overnight, diluted with EtOAc and the resulting precipitate was filtered to afford the desired compound (625 mg, 61%).

### Step 3: (2S)-2-[(3-{[(benzyloxy)carbonyl]amino}propanoyl)amino]-6-[(tert-butoxycarbonyl)amino]hexanoic acid

To a solution of methyl-(2S)-2-[(3-{[(benzyloxy)carbonyl]amino}propanoyl)amino]-6-[(tert-butoxycarbonyl)amino]hexanoate (EXAMPLE 1, Step 1; 1.0 g, 2.15 mmol) in THF (8 mL) and MeOH (4 mL) was added 2N NaOH (2.2 mL, 4.4 mmol). The mixture was stirred 1 hr at room temperature, diluted with EtOAc and quenched with saturated aqueous NH₄Cl. The organic phase was extracted, washed with brine, dried over Na₂SO₄, filtered and concentrated to afford the desired compound (910 mg, 94%) which was used without any further purification.

### Step 4: methyl(8S,11S,14S)-14-[{(2S)-2-[(3-{[(benzyloxy)carbonyl]amino}propanoyl)amino]-6-[(tert-butoxycarbonyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate

The title compound was prepared according to the procedure described in EXAMPLE 1, Step 6, but using the acid (2S)-2-[(3-{[(benzyloxy)carbonyl]amino}propanoyl)amino]-6-[(tert-butoxycarbonyl)amino]hexanoic acid from Step 4.

### Step 5: methyl(8S,11S,14S)-14-[{(2S)-2-[(3-aminopropanoyl)amino]-6-[(tert-butoxycarbonyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate

The title compound was prepared according to the procedure described in EXAMPLE 1, Step 2, but using methyl(8S,11S,14S)-14-[{(2S)-2-[(3-{[(benzyloxy)carbonyl]amino}propanoyl)amino]-6-[(tert-butoxycarbonyl)amino]hexanoyl}(methyl)amino]-3,18-dimethoxy-11-methyl-10,13-dioxo-9,12-diazatricyclo[13.3.1.1^{2,6}]icosa-1(19),2(20),3,5,15,17-hexaene-8-carboxylate from Step 4. The title compound was converted to EXAMPLE 10 following the procedures described in EXAMPLE 1, Steps 1, 6 and 7. Upon careful examination of NMR spectrum it was found that the lysine residue had racemized. The resulting diastereoisomer were inseparable by normal or reverse phase chromatography. Racemization was found to occur during the coupling reaction described in Step 4. LRMS (ESI): (calc) 900.44 (found) 901.45 (MH⁺)

### Example 11

### Method B

### 4-[{2-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-2-oxoethyl}(3-{[(4′-propylbiphenyl-4-yl)carbonyl]amino}propanoyl)amino]butan-1-aminium trifluoroacetate

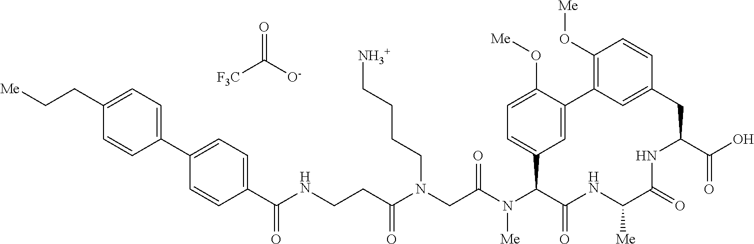 [SMILES: CCCc1ccc(-c2ccc(C(=O)NCCC(=O)N(CCCC[NH3+])CC(=O)N(C)[C@@H]3C(=O)N[C@@H](C)C(=O)N[C@H](C(=O)O)Cc4ccc(OC)c(c4)-c4cc3ccc4OC)cc2)cc1 ; O=C([O-])C(F)(F)F]

### Step 1: methyl({4-[(tert-butoxycarbonyl)amino]butyl}amino)acetate

To a cold (0° C.) solution of tert-butyl (4-aminobutyl)carbamate (3.06 g, 16.3 mmol) and DIPEA (0.502 ml, 2.88 mmol) in THF (45 mL) was added methyl bromoacetate (0.50 ml, 5.43 mmol) slowly. The mixture was stirred at 0° C. for 1 hr then allowed to warm to room temperature and stirred for 16 hr. To the resulting reaction mixture was added brine and extracted with CH₂Cl₂. The resulting mixture was passed through a phase separator and the organic layer was concentrated. The crude residue was purified by ISCO (silica gel, 24 g cartridge) eluting with CH₂Cl₂/MeOH/(0 to 20% over 40 min) to afford the desired material (591 mg, 42%) as a colorless oil.

EXAMPLE 11 was prepared in 6 additional steps as described in EXAMPLE 1, Step 2 to 7 starting from methyl({4-[(tert-butoxycarbonyl)amino]butyl}amino)acetate (Step 1). LRMS (ESI): (calc) 862.43 (found) 863.5 (MH⁺)

### Example 12

### Method C

### (8S,11S,14S)-3,18-dihydroxy-11-methyl-14-(methyl{[(2S)-1-(3-{[(4′-propylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid

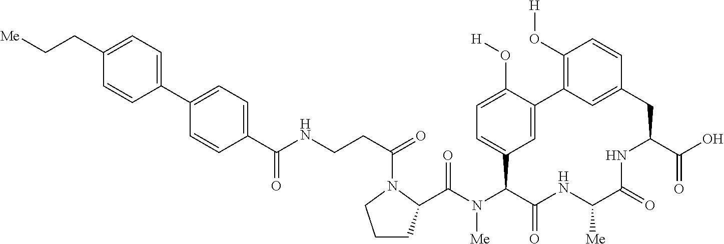 [SMILES: CCCc1ccc(-c2ccc(C(=O)NCCC(=O)N3CCC[C@H]3C(=O)N(C)[C@@H]3C(=O)N[C@@H](C)C(=O)N[C@H](C(=O)O)Cc4ccc(O)c(c4)-c4cc3ccc4O)cc2)cc1]

The title compound was prepared according to the procedure described in EXAMPLE 3 using (2S)-1-(tert-butoxycarbonyl)pyrrolidine-2-carboxylic acid in Step 3. The BOC protective group was remove as described in Step 6. EXAMPLE 12 was purified at the last step by reverse phase HPLC eluting with increment amount of CH₃CN in water (0.1% TFA).

LRMS (ESI): (calc) 803.35 (found) 804.2 (MH⁺)

### Example 13

### Method C

### (8S,11S,14S)-3,18-dimethoxy-11-methyl-14-(methyl{[(2R)-1-(3-{[(4′-propylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid

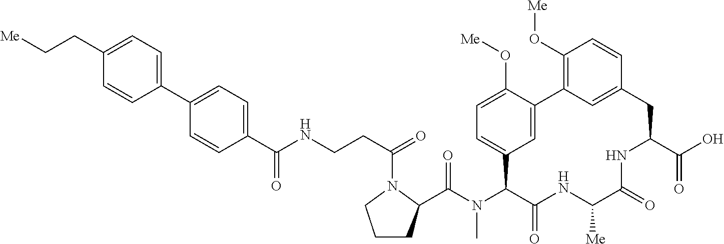 [SMILES: CCCc1ccc(-c2ccc(C(=O)NCCC(=O)N3CCC[C@@H]3C(=O)N(C)[C@@H]3C(=O)N[C@@H](C)C(=O)N[C@H](C(=O)O)Cc4ccc(OC)c(c4)-c4cc3ccc4OC)cc2)cc1]

The title compound was prepared according to the procedure described in EXAMPLE 3 using (2R)-1-(tert-butoxycarbonyl)pyrrolidine-2-carboxylic acid in Step 3. In the last step of the sequence, the ester intermediate was saponified according to the procedure described in EXAMPLE 1, Step 4. No purification was necessary to obtain clean material.

### Example 14

### (8S,11S,14S)-3,18-dihydroxy-11-methyl-14-(methyl{[(2R)-1-{3-[(6-decyl-2-naphthoyl)amino]propanoyl}pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid

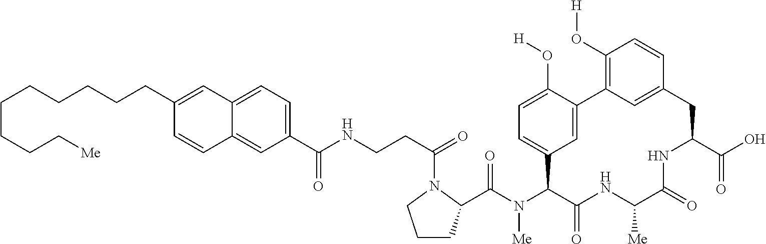 [SMILES: CCCCCCCCCCc1ccc2cc(C(=O)NCCC(=O)N3CCC[C@H]3C(=O)N(C)[C@@H]3C(=O)N[C@@H](C)C(=O)N[C@H](C(=O)O)Cc4ccc(O)c(c4)-c4cc3ccc4O)ccc2c1]

### Step 1: methyl 6-decyl-2-naphthoate

To a suspension of methyl 6-bromo-2-naphthoate (15.0 g, 56.6 mmol), N-decylboronic acid (17.9 g, 96 mmol) and potassium carbonate (23.5 g, 170 mmol) in degassed toluene (283 ml) were added 2-dicyclohexylphosphino-2′,4′,6′-tri-isopropyl-1,1′-biphenyl (7.09 g, 14.87 mmol) and Palladium (II) acetate (1.65 g, 7.36 mmol). The reaction mixture was stirred at 90° C. under nitrogen atmosphere for 16 hrs. The resulting black reaction mixture was cooled to room temperature, filtered through a pad of celite and the filtrate was concentrated. The crude residue was then filtered through a pad of silica gel eluting with 40% EtOAc in Hex. The filtrate was concentrated under reduced pressure and the crude product was purified by ISCO (silica gel, 330 g cartridge) eluting with Hex/EtOAc (0% to 7%) to afford the desired material (19.9 g, 97%) as a brown solid.

### Step 2: 6-decyl-2-naphthoic acid

To a solution of methyl 6-decyl-2-naphthoate (17.9 g, 55.0 mmol) in THF/MeOH (3900 mL, 1/1) was added 2M LiOH (90 ml, 180 mmol). The resulting mixture was stirred at 60° C. for 3 hours and the organic solvents were removed in vacuo to one third volume. The resulting precipitate was filtered and then it was suspended into aqueous 10% HCl. The white solid was filtered, washed with water and dried under reduced pressure to afford the title compound (14.0 g, 81%) as a white solid.

EXAMPLE 14 was prepared according to the procedure described in EXAMPLE 3 using 6-decyl-2-naphthoic acid in Step 1 and (2S)-1-(tert-butoxycarbonyl)pyrrolidine-2-carboxylic acid in Step 3. The BOC protective group was remove as described in Step 6. EXAMPLE 14 was purified in the last step by ISCO (silica gel) eluting with CH₂Cl₂/MeOH/1% AcOH (5 to 25%) to afford the title compound as a white solid. LRMS (EST): (calc) 875.45 (found) 876.25 (MH+)

### Example 15

### (5S)-6-[[(7S,10S,13S)-13-carboxy-3,18-dihydroxy-10-methyl-8,11-dioxo-9,12-diazatricyclo[13.3.1.12,6]icosa-1(19),2(20),3,5,15,17-hexaen-7-yl](methyl)amino]-6-oxo-5-[(3-{[4-(8-phenyloctyl)benzoyl]amino}propanoyl)amino]hexan-1-aminium

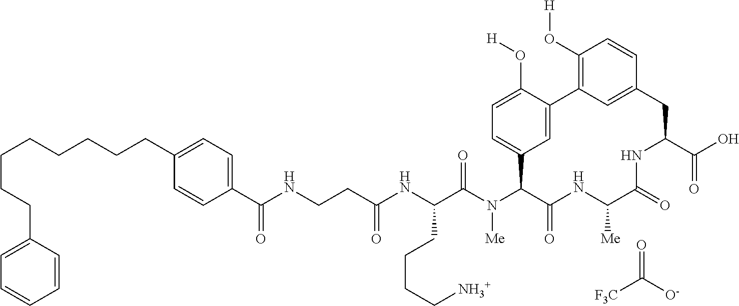 [SMILES: C[C@@H]1NC(=O)[C@@H](N(C)C(=O)[C@H](CCCC[NH3+])NC(=O)CCNC(=O)c2ccc(CCCCCCCCc3ccccc3)cc2)c2ccc(O)c(c2)-c2cc(ccc2O)C[C@@H](C(=O)O)NC1=O ; O=C([O-])C(F)(F)F]

### Step 1: oct-7-yn-1-ylbenzene

To a solution of lithium acetylide 1-2 diaminoethyl complex (1.86 g, 20.2 mmol) in DMSO (80 mL) at 10° C. was added dropwise over 5 min (6-bromohexyl)benzene (3.25 g, 13.5 mmol). The resulting mixture was stirred at 10° C. for 45 min then warm to room temperature. The mixture was poured into water, extracted with Et₂O (3×). The combined organic extracts were washed with water, dried over MgSO₄, filtered and concentrated to afford the title compound (2.46 g, 98%) as a yellow oil.

### Step 2: methyl 4-(8-phenyloct-1-yn-1-yl)benzoate

Oct-7-yn-1-ylbenzene (2.60 g, 14.0 mmol), methyl 4-bromobenzoate (1.5 g, 6.98 mmol), CuI (0.213 g, 1.12 mmol) and Pd(PPh₃)₄ (0.645 g, 0.558 mmol) were loaded in a reaction tube and flushed with N₂. THF (5 mL) and triethylamine (2.92 ml, 20.9 mmol) were added and the resulting mixture was stirred at 60° C. for 24 hrs. The mixture was cooled room temperature, filtered through a pad of Celite and concentrated. The crude product was purified by Isco (silica gel, 12 g cartridge), eluting with Hex/EtOAc (0 to 5%) to afford the desired material (2.23 g, 99%) as a yellow oil.

### Step 3: methyl 4-(8-phenyloctyl)benzoate

A mixture of methyl 4-(8-phenyloct-1-yn-1-yl)benzoate (2.55 g, 7.96 mmol) and Pd/C 10% (0.85 g) in MeOH (16 mL) was stirred under an atmosphere of hydrogen for 16 hrs. The resulting mixture was filtered on a pad of Celite and was concentrated to afford the desired compound (2.48 g, 96%) as a pale yellow oil. It was used in the next step without any further purification.

### Step 4: 4-(8-phenyloctyl)benzoic acid

To a solution of methyl 4-(8-phenyloctyl)benzoate (168 mg, 0.518 mmol) in MeOH/THF (2 mL, 1/1) was added NaOH 1N (7.77 mL, 7.77 mmol). The mixture was stirred at room temperature for 24 hr. The resulting solution was concentrated; the residual aqueous solution was acidified using HCl 1N to pH 2 and extracted with CH₂Cl₂. The organic extract was dried over Na₂SO₄, filtered and concentrated to yield the title compound (160 mg, 100%) as a colorless oil. The material was used in the next step without any further purification.

EXAMPLE 15 was prepared according to the procedure described in EXAMPLE 3 using 4-(8-phenyloctyl)benzoic acid Step 1. EXAMPLE 15 was purified by reverse phase HPLC on a MAX-RP column eluting with CH₃CN/H₂O (0.1% TFA) with incrementing amount of CH₃CN from 35 to 60%. LRMS (ESI): (calc) 904.47 (found) 905.50 (MH+).

EXAMPLE 16

### Method C

### (5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-[(3-{[(4′-nonylbiphenyl-4-yl)carbonyl]amino}propanoyl)amino]-6-oxohexan-1-aminium trifluoroacetate

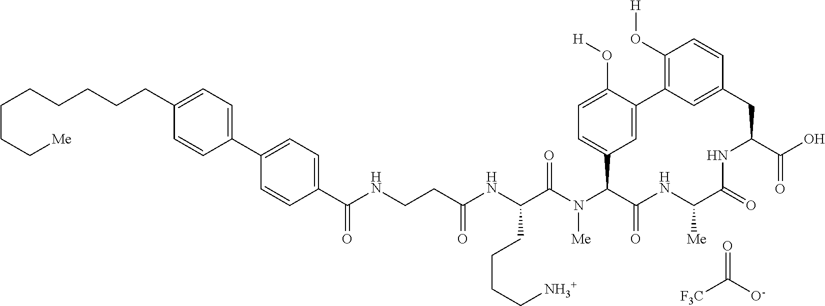 [SMILES: CCCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F]

### Step 1: methyl 4′-non-1-yn-1-ylbiphenyl-4-carboxylate

To a solution of non-1-yne (1.26 g, 10.2 mmol) and methyl 4′-bromobiphenyl-4-carboxylate (1.48 g, 5.08 mmol) in DMF (20 mL) was added DIPEA (2.66 mL, 15.3 mmol) and CuI (0.194 g, 1.02 mmol). After several purges with nitrogen Pd(PPh₃)₄ (0.292 g, 0.25 mmol) was added and the mixture was stirred at 80° C. overnight. The resulting reaction mixture was cooled to room temperature and diluted with EtOAc. The organic phase was washed with HCl 10%, brine, dried over MgSO₄, filtered and concentrated. The mixture was purified on ISCO (silica gel, 80 g cartridge) eluting with Hex/EtOAc (0 to 10%). The product was then re-crystallized in hot hexanes to afford the desired material (575 mg, 34%) as a white solid.

### Step 2: 4′-nonylbiphenyl-4-carboxylic acid

A mixture of methyl 4′-non-1-yn-1-ylbiphenyl-4-carboxylate (575 mg, 1.72 mmol) and Pd/C 10% (183 mg) in MeOH (10 mL)/THF (1 mL) was stirred under an atmosphere of hydrogen for 16 hrs. The resulting mixture was filtered on a pad of Celite and was concentrated to afford methyl 4′-nonylbiphenyl-4-carboxylate (550 mg, 95%) as a pale yellow oil. The latter was saponified as described in EXAMPLE 15, Step 4 to yield the title compound.

EXAMPLE 16 was prepared according to the procedure described in EXAMPLE 3 using 4′-nonylbiphenyl-4-carboxylic acid in Step 1. EXAMPLE 16 was purified by reverse phase HPLC on a MAX-RP column eluting with CH₃CN/H₂O (0.1% TFA) with incrementing amount of CH₃CN from 40 to 80%. LRMS (ESI): (calc) 918.49 (found) 919.45 (MH⁺).

### Example 17

### Method C

### (5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-({3-[(6-decyl-2-naphthoyl)amino]propanoyl}amino)-6-oxohexan-1-aminium trifluoroacetate

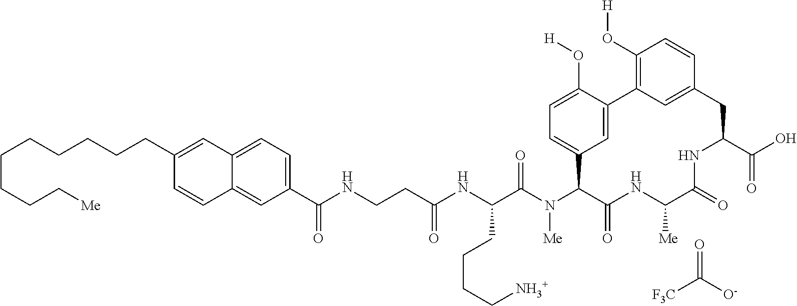 [SMILES: CCCCCCCCCCc1ccc2cc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)ccc2c1 ; O=C([O-])C(F)(F)F]

EXAMPLE 17 was prepared according to the procedure described in EXAMPLE 3 using 6-decyl-2-naphthoic acid (EXAMPLE 14, Step 2) in Step 1. EXAMPLE 15 was purified by reverse phase HPLC on a MAX-RP column eluting with CH₃CN/H₂O (0.1% TFA) with incrementing amount of CH₃CN from 40 to 80%. LRMS (ESI): (calc) 906.49 (found) 907.45 (MH⁺).

### Example 18

### Method C

### (8S,11S,14S)-3,18-dihydroxy-11-methyl-14-(methyl{[(2)-1-(3-{[(4′-nonylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid

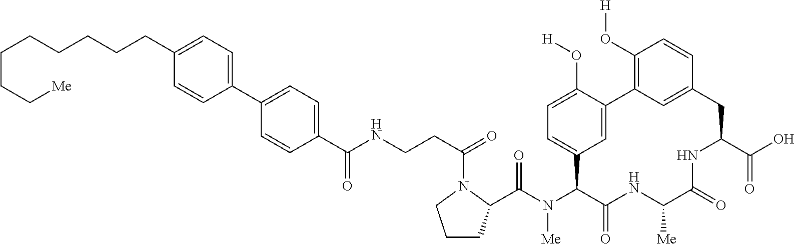 [SMILES: CCCCCCCCCc1ccc(-c2ccc(C(=O)NCCC(=O)N3CCC[C@H]3C(=O)N(C)[C@@H]3C(=O)N[C@@H](C)C(=O)N[C@H](C(=O)O)Cc4ccc(O)c(c4)-c4cc3ccc4O)cc2)cc1]

EXAMPLE 18 was prepared according to the procedure described in EXAMPLE 3 using 4′-nonylbiphenyl-4-carboxylic acid (EXAMPLE 16, Step 4) in Step 1 and (2S)-1-(tert-butoxycarbonyl)pyrrolidine-2-carboxylic acid in Step 3. The BOC protective group was remove as described in Step 6. EXAMPLE 18 was purified by reverse phase HPLC on a MAX-RP column eluting with CH₃CN/H₂O (0.1% TFA) with incrementing amount of CH₃CN from 40 to 80%. LRMS (EST): (calc) 887.45 (found) 888.40 (MH⁺).

### Example 19

### Method C

### (8S,11S,14S)-3,18-dihydroxy-11-methyl-14-(methyl{[(2)-1-(3-{[(4′-nonylbiphenyl-4-yl)carbonyl]amino}propanoyl)azetidin-2-yl]carbonyl}amino)-10,13-dioxo-9,12-diazatricyclo[13.3.1.1^{2,6}]icosa-1(19),2(20),3,5,15,17-hexaene-8-carboxylic acid

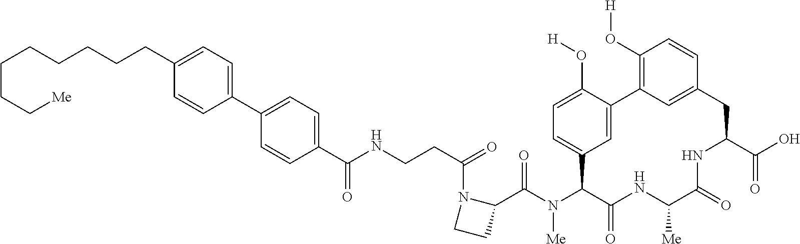 [SMILES: CCCCCCCCCc1ccc(-c2ccc(C(=O)NCCC(=O)N3CC[C@H]3C(=O)N(C)[C@@H]3C(=O)N[C@@H](C)C(=O)N[C@H](C(=O)O)Cc4ccc(O)c(c4)-c4cc3ccc4O)cc2)cc1]

EXAMPLE 19 was prepared according to the procedure described in EXAMPLE 3 using 4′-nonylbiphenyl-4-carboxylic acid (EXAMPLE 16, Step 4) in Step 1 and (2S)-1-(tert-butoxycarbonyl)azetidine-2-carboxylic acid in Step 3. The BOC protective group was remove as described in Step 6. EXAMPLE 19 was purified by reverse phase HPLC on a MAX-RP column eluting with CH₃CN/H₂O (0.1% TFA) with incrementing amount of CH₃CN from 40 to 80%. LRMS (ESI): (calc) 873.43 (found) 874.30 (MH+).

### Example 20

### Method C

### (8S,11S,14S)-3,18-dihydroxy-11-methyl-14-(methyl[(2R)-1-{3-[(6-decyl-2-naphthoyl)amino]propanoyl azetidin-2-yl]carbonyl}amino)-10,13-dioxo-9,12-diazatricyclo[13.3.1.1^{2,6}]icosa-1(19),2(20),3,5,15,17-hexaene-8-carboxylic acid

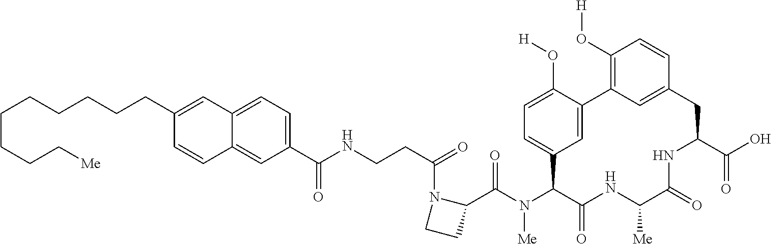 [SMILES: CCCCCCCCCCc1ccc2cc(C(=O)NCCC(=O)N3CC[C@H]3C(=O)N(C)[C@@H]3C(=O)N[C@@H](C)C(=O)N[C@H](C(=O)O)Cc4ccc(O)c(c4)-c4cc3ccc4O)ccc2c1]

EXAMPLE 20 was prepared according to the procedure described in EXAMPLE 3 using 6-decyl-2-naphthoic acid (EXAMPLE 14, Step 2) in Step 1 and (2S)-1-(tert-butoxycarbonyl)azetidine-2-carboxylic acid in Step 3. The BOC protective group was remove as described in Step 6. EXAMPLE 20 was purified by reverse phase HPLC on a MAX-RP column eluting with CH₃CN/H₂O (0.1% TFA) with incrementing amount of CH₃CN from 40 to 80%. LRMS (EST): (calc) 861.43 (found) 862.30 (MH⁺).

### Examples 21-67

EXAMPLES 21-67 were prepared using methods analogous to those described in the preceding Schemes and Examples.

| EXAM- | | LRMS |
|---|---|---|
| PLE | | (ESI) |
| 21 | 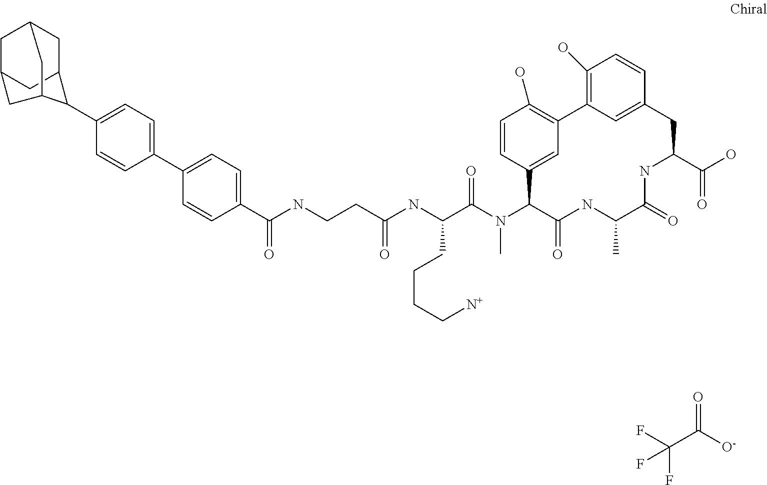 [SMILES: C[C@@H]1NC(=O)[C@@H](N(C)C(=O)[C@H](CCCC[NH3+])NC(=O)CCNC(=O)c2ccc(-c3ccc(C4C5CC6CC(C5)CC4C6)cc3)cc2)c2ccc(O)c(c2)-c2cc(ccc2O)C[C@@H](C(=O)O)NC1=O ; O=C([O-])C(F)(F)F] | 1041.144 |
| 22 | 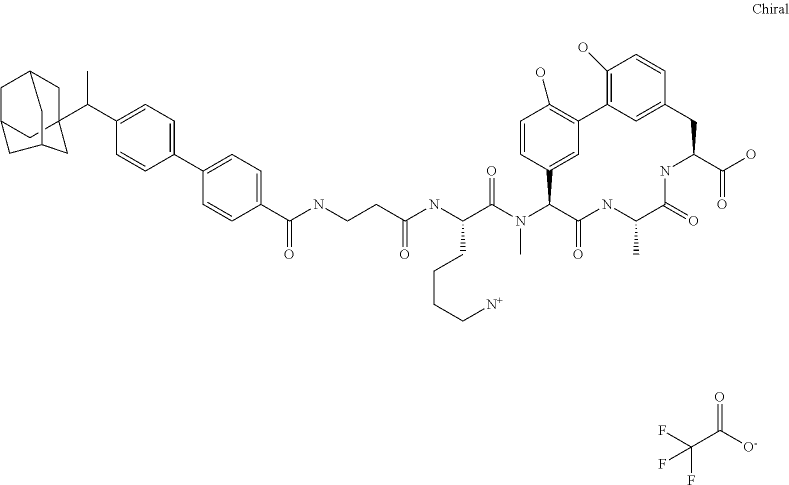 [SMILES: CC(c1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1)C12CC3CC(CC(C3)C1)C2 ; O=C([O-])C(F)(F)F] | 1069.198 |
| 23 | 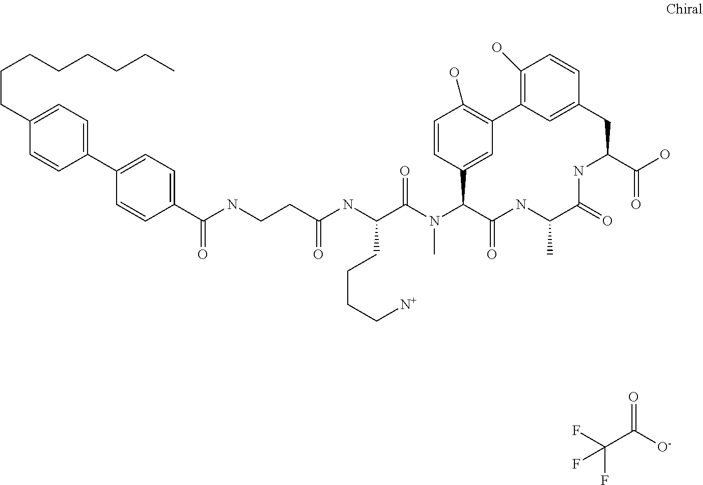 [SMILES: CCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 1019.138 |
| 24 | 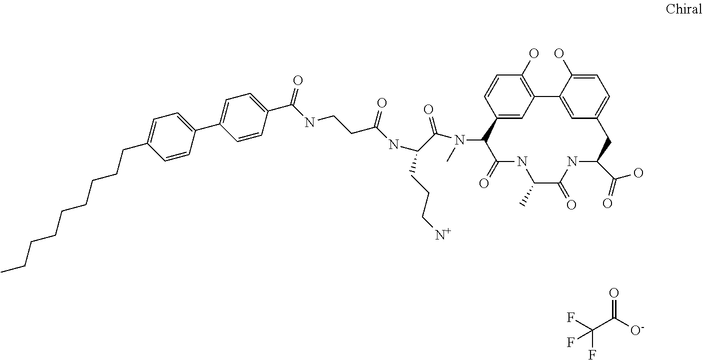 | 1019.138 |
| 25 | 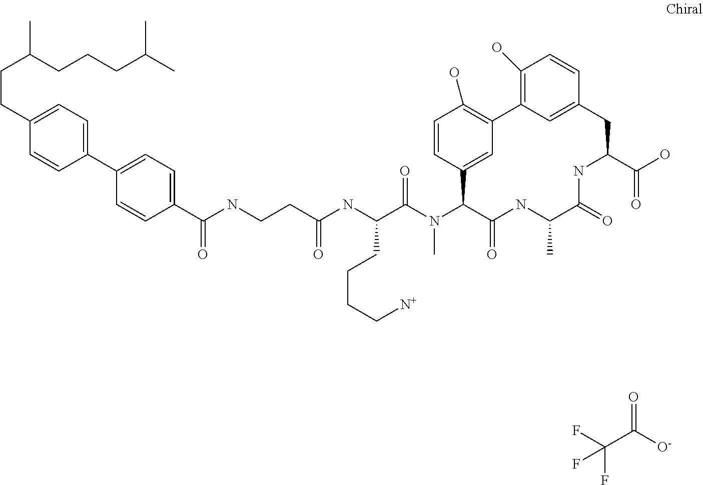 [SMILES: CC(C)CCCC(C)CCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 1047.192 |
| 26 | 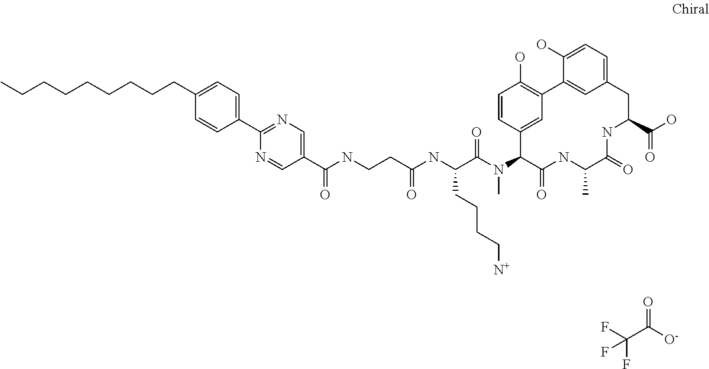 [SMILES: CCCCCCCCCc1ccc(-c2ncc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cn2)cc1 ; O=C([O-])C(F)(F)F] | 1035.14 |
| 27 | 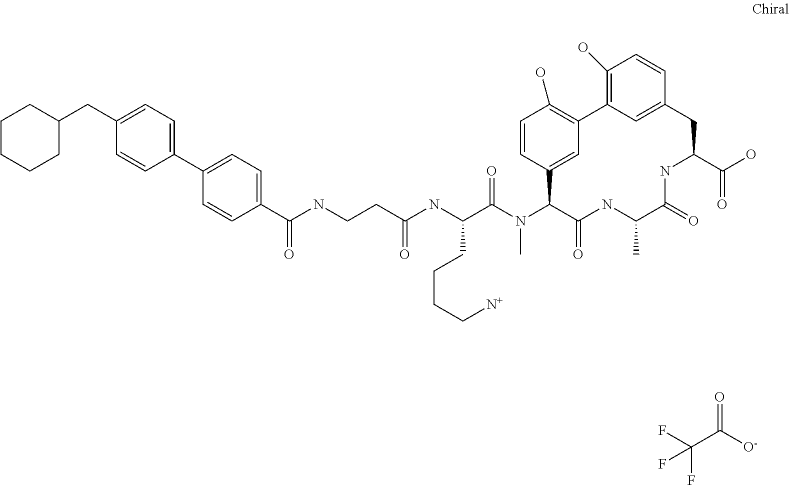 [SMILES: C[C@@H]1NC(=O)[C@@H](N(C)C(=O)[C@H](CCCC[NH3+])NC(=O)CCNC(=O)c2ccc(-c3ccc(CC4CCCCC4)cc3)cc2)c2ccc(O)c(c2)-c2cc(ccc2O)C[C@@H](C(=O)O)NC1=O ; O=C([O-])C(F)(F)F] | 1003.094 |
| 28 | 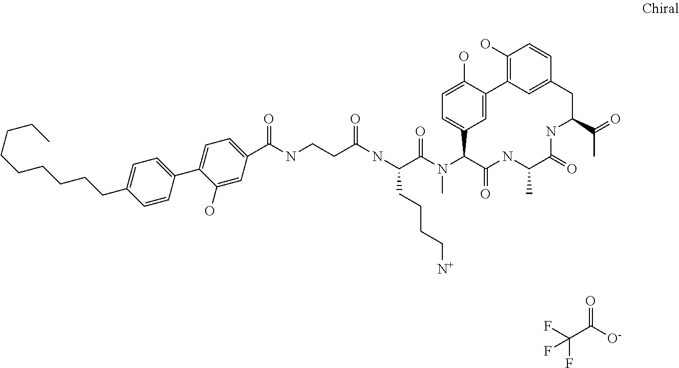 [SMILES: CCCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(C)=O)Cc4ccc(O)c(c4)-c4cc3ccc4O)cc2O)cc1 ; O=C([O-])C(F)(F)F] | 1049.164 |
| 29 | 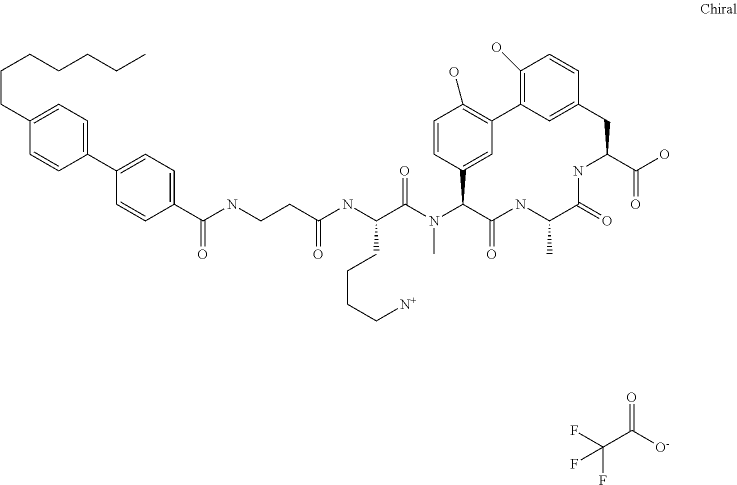 [SMILES: CCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 1005.11 |
| 30 | 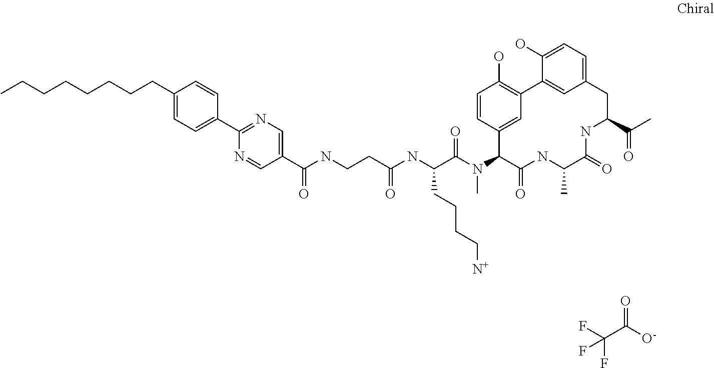 [SMILES: CCCCCCCCc1ccc(-c2ncc(C(=O)NCCC(=O)N[C@@H](CCCC[NH3+])C(=O)N(C)[C@@H]3C(=O)N[C@@H](C)C(=O)N[C@H](C(C)=O)Cc4ccc(O)c(c4)-c4cc3ccc4O)cn2)cc1 ; O=C([O-])C(F)(F)F] | 1021.113 |
| 31 | 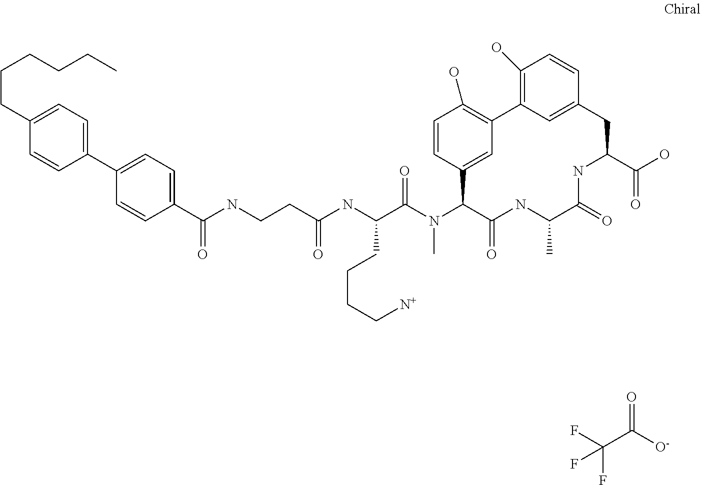 [SMILES: CCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 991.083 |
| 32 | 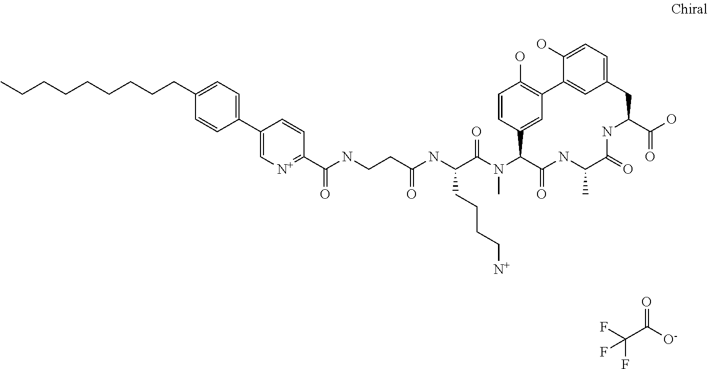 [SMILES: CCCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)[nH+]c2)cc1 ; O=C([O-])C(F)(F)F] | 1148.176 |
| 33 | 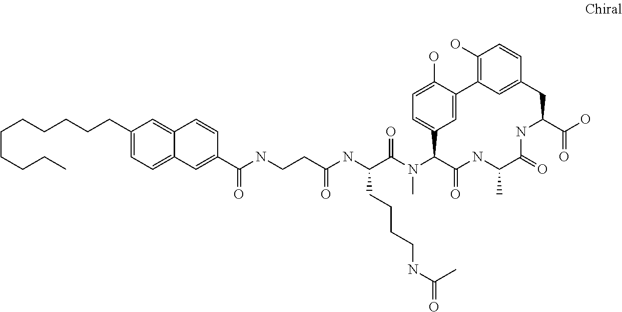 [SMILES: CCCCCCCCCCc1ccc2cc(C(=O)NCCC(=O)N[C@@H](CCCCNC(C)=O)C(=O)N(C)[C@@H]3C(=O)N[C@@H](C)C(=O)N[C@H](C(=O)O)Cc4ccc(O)c(c4)-c4cc3ccc4O)ccc2c1] | 949.167 |
| 34 | 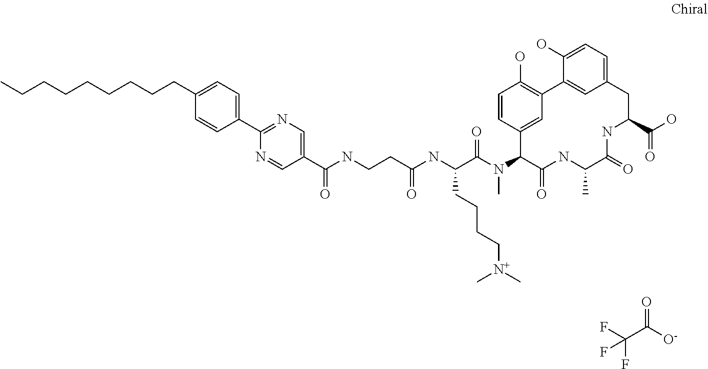 [SMILES: CCCCCCCCCc1ccc(-c2ncc(C(=O)NCCC(=O)N[C@@H](CCCC[NH+](C)C)C(=O)N(C)[C@@H]3C(=O)N[C@@H](C)C(=O)N[C@H](C(=O)O)Cc4ccc(O)c(c4)-c4cc3ccc4O)cn2)cc1 ; O=C([O-])C(F)(F)F] | 1063.194 |
| 35 | 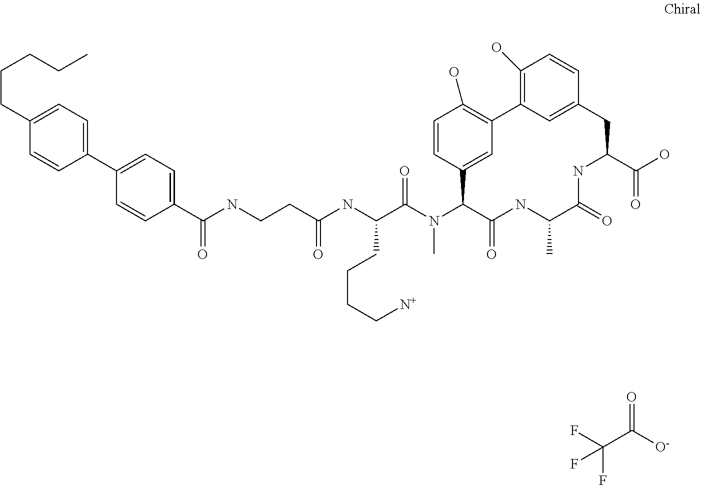 [SMILES: CCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 977.056 |
| 36 | 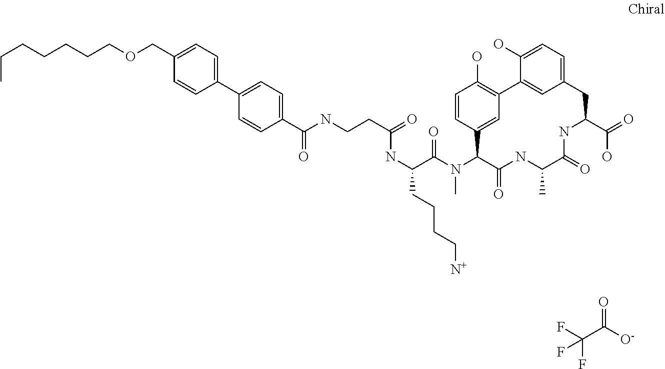 [SMILES: CCCCCCCOCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 1035.137 |
| 37 | 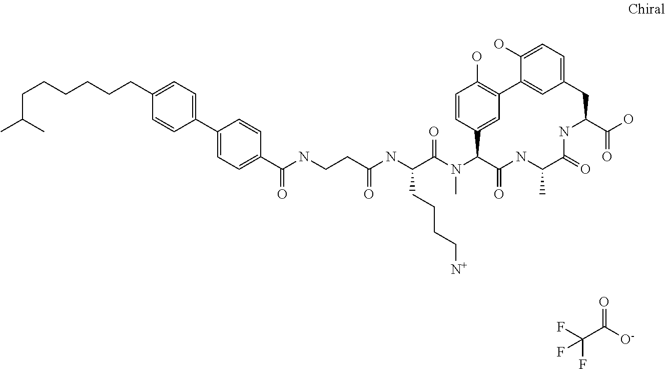 [SMILES: CC(C)CCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 1033.165 |
| 38 | 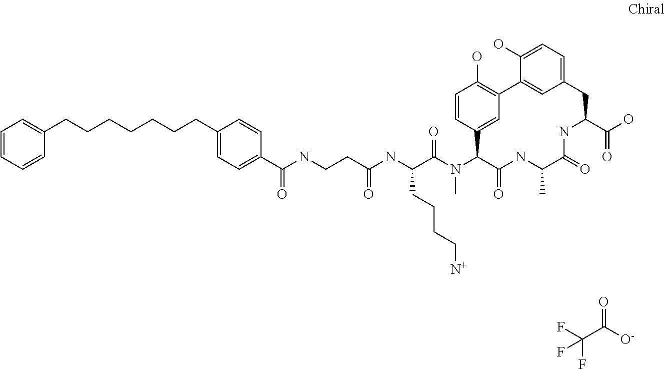 [SMILES: C[C@@H]1NC(=O)[C@@H](N(C)C(=O)[C@H](CCCC[NH3+])NC(=O)CCNC(=O)c2ccc(CCCCCCCc3ccccc3)cc2)c2ccc(O)c(c2)-c2cc(ccc2O)C[C@@H](C(=O)O)NC1=O ; O=C([O-])C(F)(F)F] | 1005.11 |
| 39 | 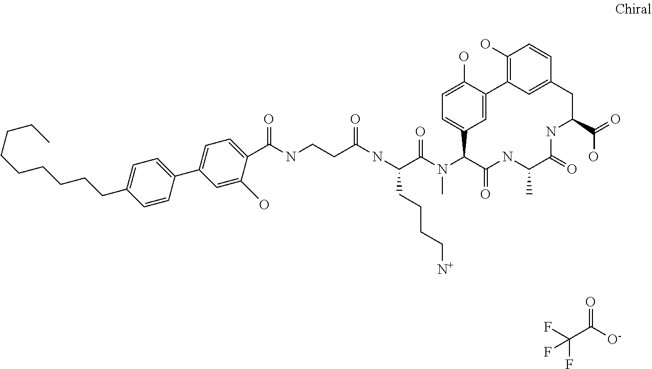 [SMILES: CCCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)c(O)c2)cc1 ; O=C([O-])C(F)(F)F] | 1049.164 |
| 40 | 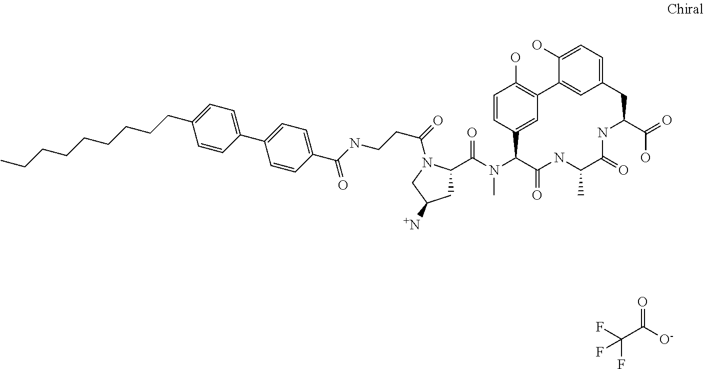 [SMILES: CCCCCCCCCc1ccc(-c2ccc(C(=O)NCCC(=O)N3C[C@H]([NH3+])C[C@H]3C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 1017.122 |
| 41 | 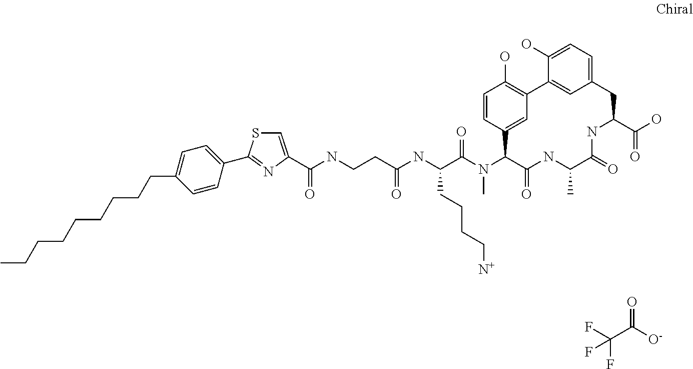 [SMILES: CCCCCCCCCc1ccc(-c2nc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cs2)cc1 ; O=C([O-])C(F)(F)F] | 1040.178 |
| 42 | 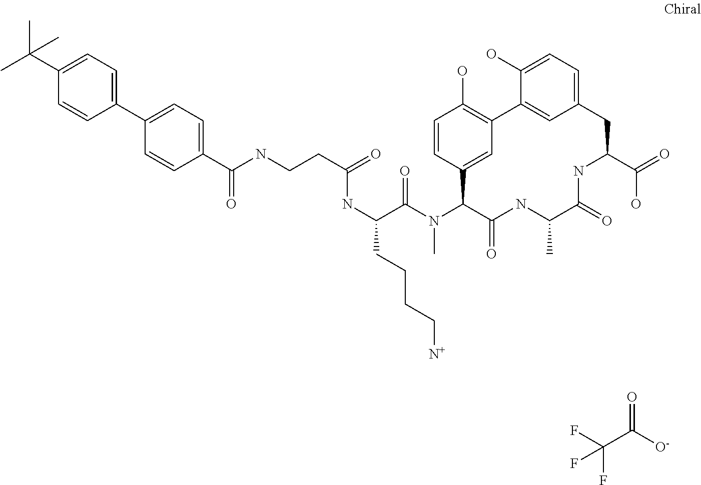 [SMILES: C[C@@H]1NC(=O)[C@@H](N(C)C(=O)[C@H](CCCC[NH3+])NC(=O)CCNC(=O)c2ccc(-c3ccc(C(C)(C)C)cc3)cc2)c2ccc(O)c(c2)-c2cc(ccc2O)C[C@@H](C(=O)O)NC1=O ; O=C([O-])C(F)(F)F] | 963.029 |
| 43 | 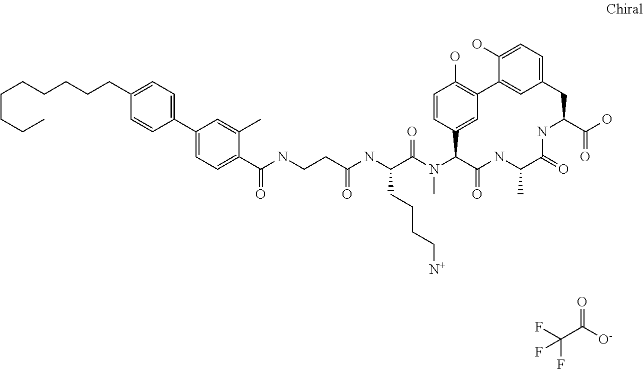 [SMILES: CCCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)c(C)c2)cc1 ; O=C([O-])C(F)(F)F] | 1047.192 |
| 44 | 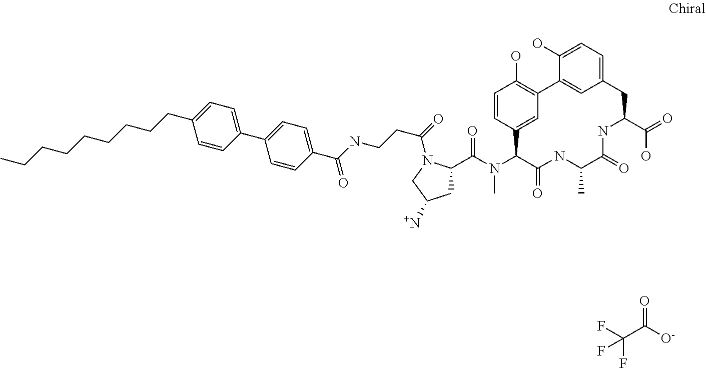 [SMILES: CCCCCCCCCc1ccc(-c2ccc(C(=O)NCCC(=O)N3C[C@@H]([NH3+])C[C@H]3C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 1017.122 |
| 45 | 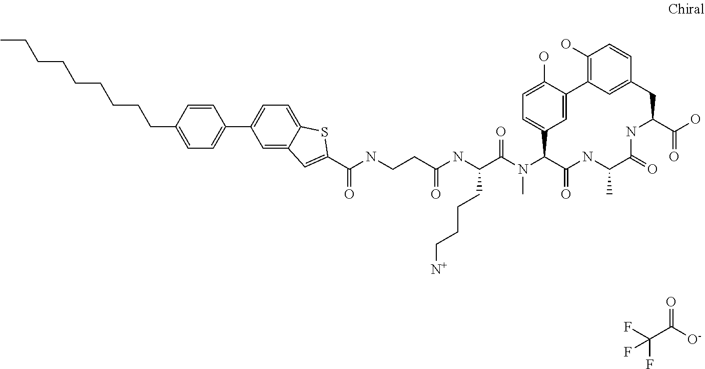 [SMILES: CCCCCCCCCc1ccc(-c2ccc3sc(C(=O)NCCC(=O)N[C@@H](CCCC[NH3+])C(=O)N(C)[C@@H]4C(=O)N[C@@H](C)C(=O)N[C@H](C(=O)O)Cc5ccc(O)c(c5)-c5cc4ccc5O)cc3c2)cc1 ; O=C([O-])C(F)(F)F] | 1089.251 |
| 46 | 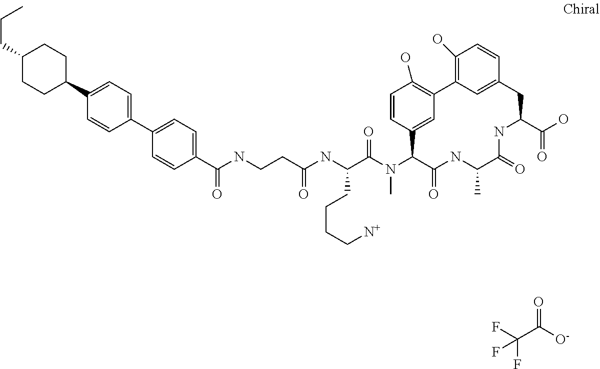 [SMILES: CCC[C@H]1CC[C@H](c2ccc(-c3ccc(C(=O)NCCC(=O)N[C@@H](CCCC[NH3+])C(=O)N(C)[C@@H]4C(=O)N[C@@H](C)C(=O)N[C@H](C(=O)O)Cc5ccc(O)c(c5)-c5cc4ccc5O)cc3)cc2)CC1 ; O=C([O-])C(F)(F)F] | 1031.149 |
| 47 | 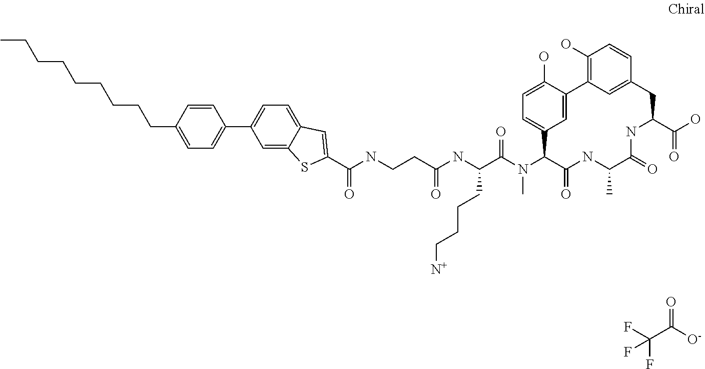 [SMILES: CCCCCCCCCc1ccc(-c2ccc3cc(C(=O)NCCC(=O)N[C@@H](CCCC[NH3+])C(=O)N(C)[C@@H]4C(=O)N[C@@H](C)C(=O)N[C@H](C(=O)O)Cc5ccc(O)c(c5)-c5cc4ccc5O)sc3c2)cc1 ; O=C([O-])C(F)(F)F] | 1089.251 |
| 48 | 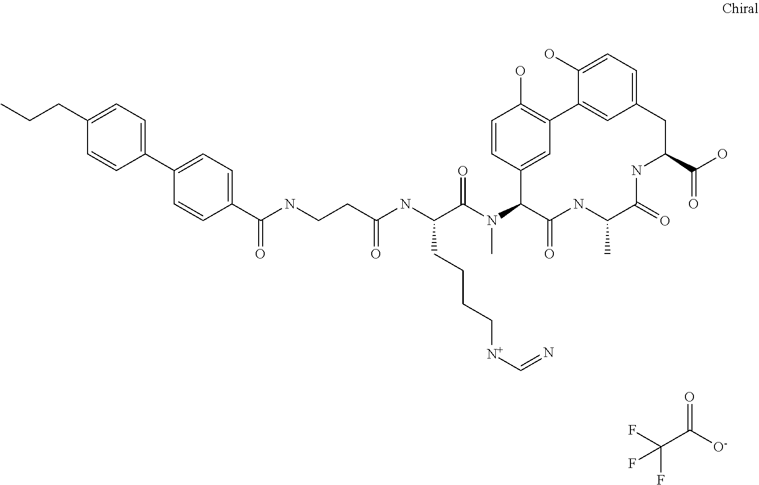 [SMILES: CCCc1ccc(-c2ccc(C(=O)NCCC(=O)N[C@@H](CCCC[NH2+]C=N)C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 976.028 |
| 49 | 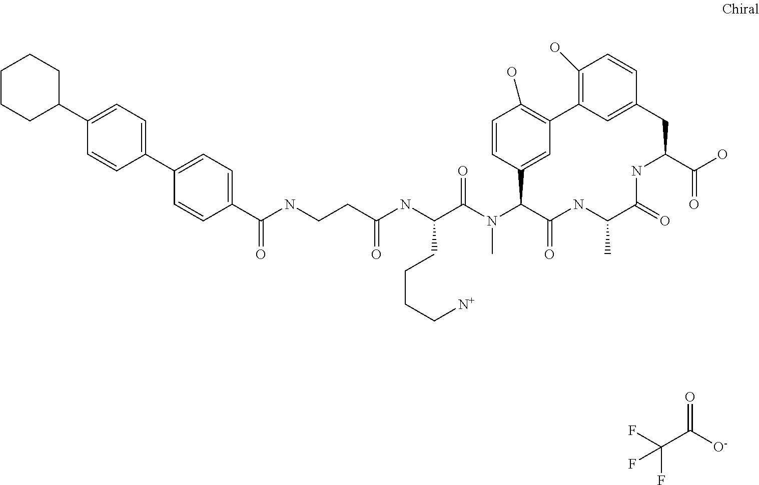 [SMILES: C[C@@H]1NC(=O)[C@@H](N(C)C(=O)[C@H](CCCC[NH3+])NC(=O)CCNC(=O)c2ccc(-c3ccc(C4CCCCC4)cc3)cc2)c2ccc(O)c(c2)-c2cc(ccc2O)C[C@@H](C(=O)O)NC1=O ; O=C([O-])C(F)(F)F] | 989.067 |
| 50 | 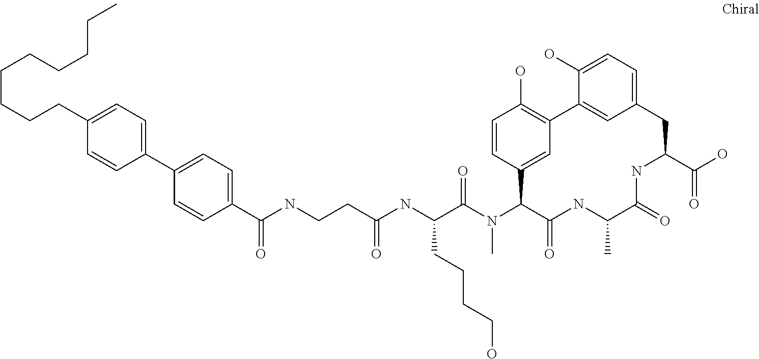 [SMILES: CCCCCCCCCc1ccc(-c2ccc(C(=O)NCCC(=O)N[C@@H](CCCCO)C(=O)N(C)[C@@H]3C(=O)N[C@@H](C)C(=O)N[C@H](C(=O)O)Cc4ccc(O)c(c4)-c4cc3ccc4O)cc2)cc1] | 920.125 |
| 51 | 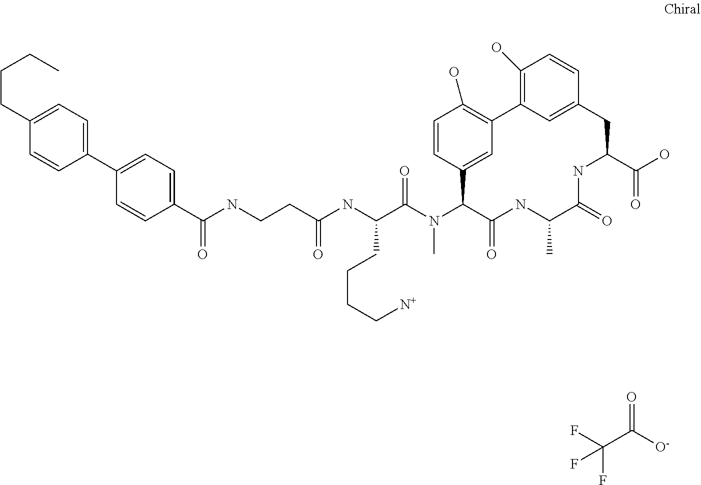 [SMILES: CCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 963.029 |
| 52 | 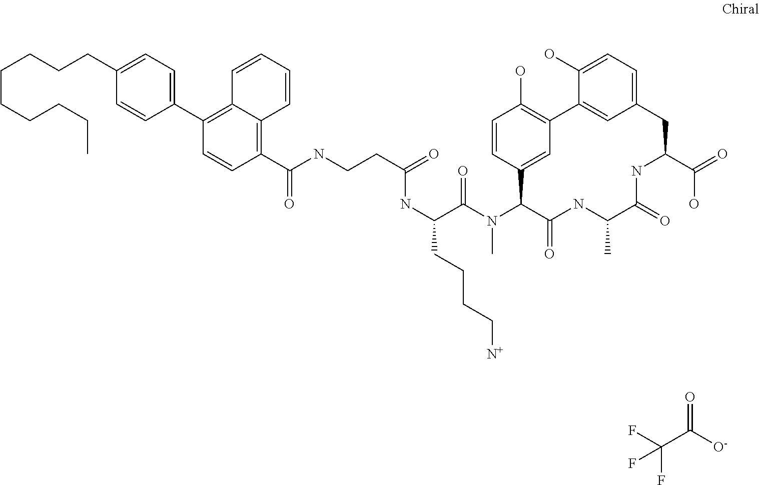 [SMILES: CCCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)c3ccccc23)cc1 ; O=C([O-])C(F)(F)F] | 1083.225 |
| 53 | 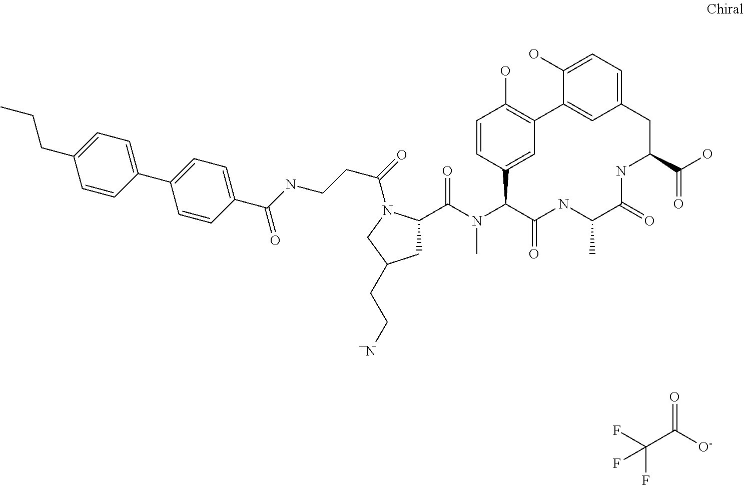 | 961.013 |
| 54 | 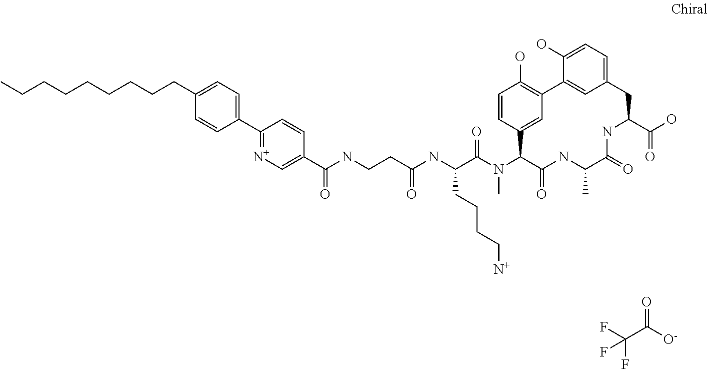 [SMILES: CCCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)c[nH+]2)cc1 ; O=C([O-])C(F)(F)F] | 1148.176 |
| 55 | 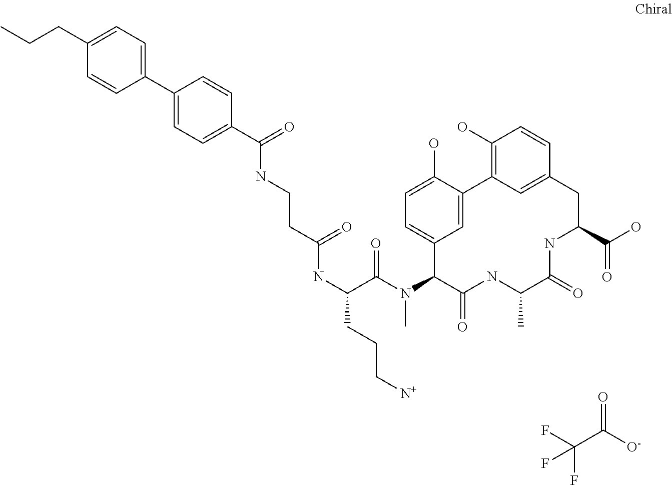 [SMILES: CCCc1ccc(-c2ccc(C(=O)NCCC(=O)N[C@@H](CCC[NH3+])C(=O)N(C)[C@@H]3C(=O)N[C@@H](C)C(=O)N[C@H](C(=O)O)Cc4ccc(O)c(c4)-c4cc3ccc4O)cc2)cc1 ; O=C([O-])C(F)(F)F] | 934.975 |
| 56 | 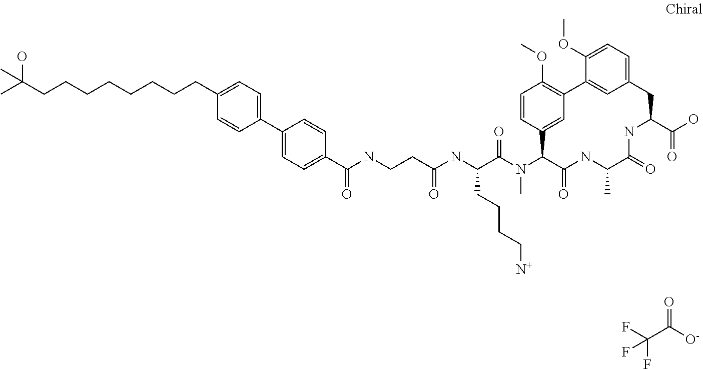 [SMILES: COc1ccc2cc1-c1cc(ccc1OC)[C@H](N(C)C(=O)[C@H](CCCC[NH3+])NC(=O)CCNC(=O)c1ccc(-c3ccc(CCCCCCCCC(C)(C)O)cc3)cc1)C(=O)N[C@@H](C)C(=O)N[C@H](C(=O)O)C2 ; O=C([O-])C(F)(F)F] | 1105.272 |
| 57 | 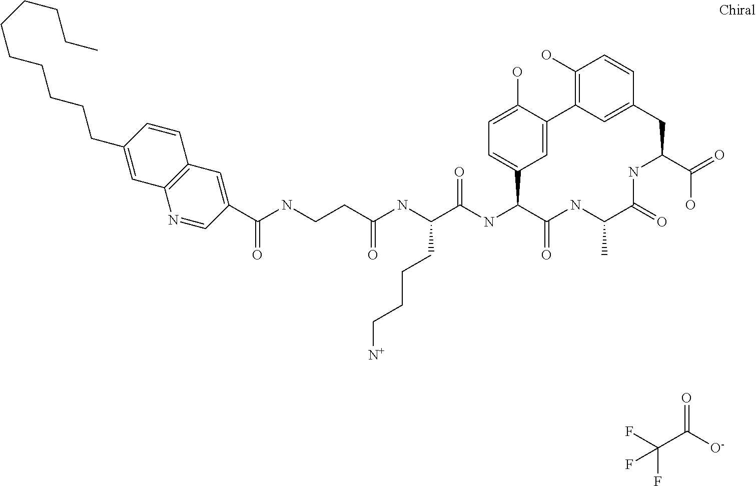 [SMILES: CCCCCCCCCCc1ccc2cc(C(=O)NCCC(=O)N[C@@H](CCCC[NH3+])C(=O)N[C@@H]3C(=O)N[C@@H](C)C(=O)N[C@H](C(=O)O)Cc4ccc(O)c(c4)-c4cc3ccc4O)cnc2c1 ; O=C([O-])C(F)(F)F] | 1022.141 |
| 58 | 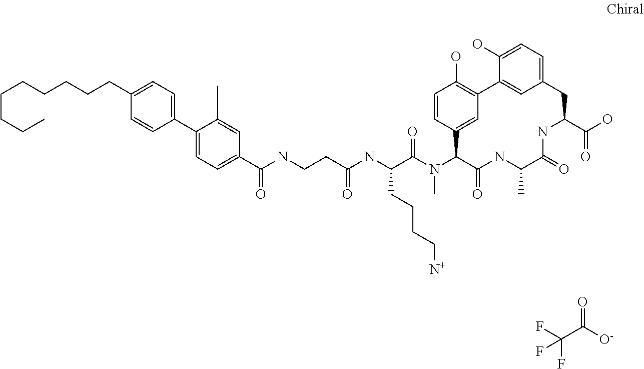 [SMILES: CCCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cc2C)cc1 ; O=C([O-])C(F)(F)F] | 1047.192 |
| 59 | 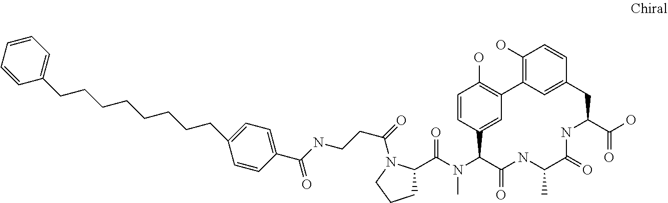 [SMILES: C[C@@H]1NC(=O)[C@@H](N(C)C(=O)[C@@H]2CCCN2C(=O)CCNC(=O)c2ccc(CCCCCCCCc3ccccc3)cc2)c2ccc(O)c(c2)-c2cc(ccc2O)C[C@@H](C(=O)O)NC1=O] | 874.056 |
| 60 | 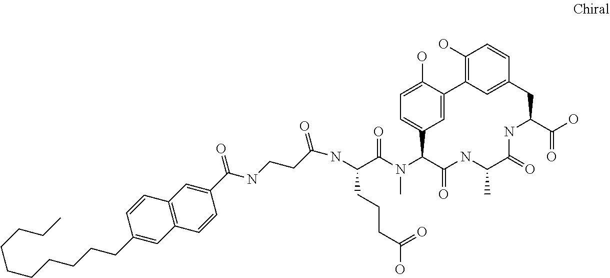 [SMILES: CCCCCCCCCCc1ccc2cc(C(=O)NCCC(=O)N[C@@H](CCCC(=O)O)C(=O)N(C)[C@@H]3C(=O)N[C@@H](C)C(=O)N[C@H](C(=O)O)Cc4ccc(O)c(c4)-c4cc3ccc4O)ccc2c1] | 922.098 |
| 61 | 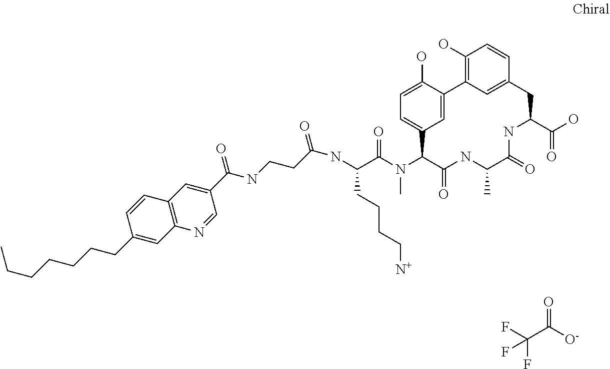 [SMILES: CCCCCCCc1ccc2cc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)cnc2c1 ; O=C([O-])C(F)(F)F] | 980.06 |
| 62 | 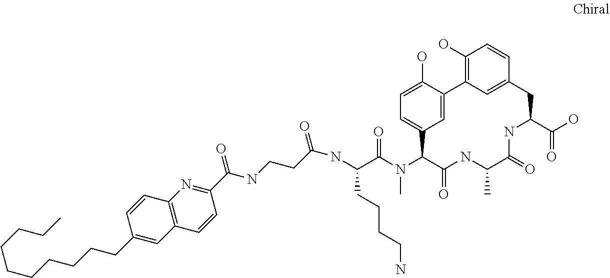 [SMILES: CCCCCCCCCCc1ccc2nc(C(=O)NCCC(=O)N[C@@H](CCCCN)C(=O)N(C)[C@@H]3C(=O)N[C@@H](C)C(=O)N[C@H](C(=O)O)Cc4ccc(O)c(c4)-c4cc3ccc4O)ccc2c1] | 908.117 |
| 63 | 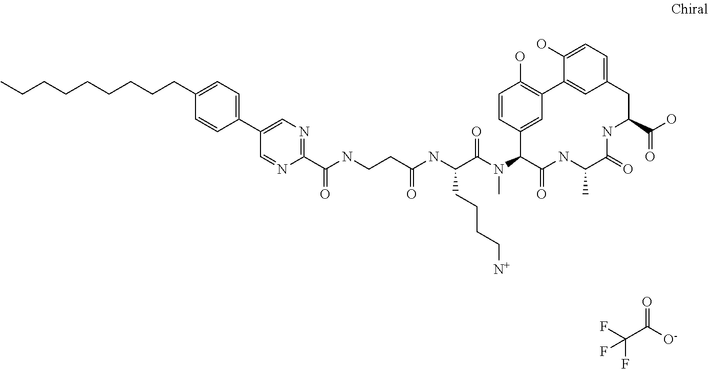 [SMILES: CCCCCCCCCc1ccc(-c2cnc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)nc2)cc1 ; O=C([O-])C(F)(F)F] | 1035.14 |
| 64 | 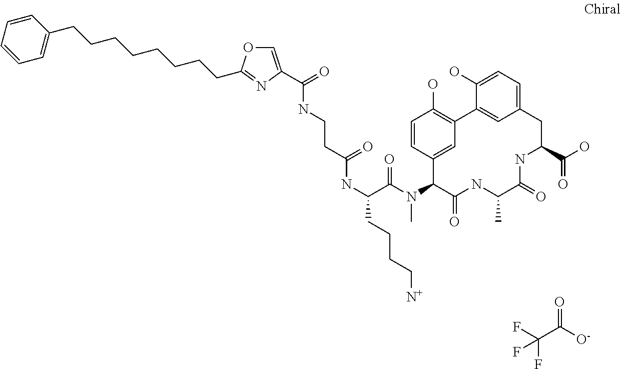 [SMILES: C[C@@H]1NC(=O)[C@@H](N(C)C(=O)[C@H](CCCC[NH3+])NC(=O)CCNC(=O)c2coc(CCCCCCCCc3ccccc3)n2)c2ccc(O)c(c2)-c2cc(ccc2O)C[C@@H](C(=O)O)NC1=O ; O=C([O-])C(F)(F)F] | 1010.086 |
| 65 | 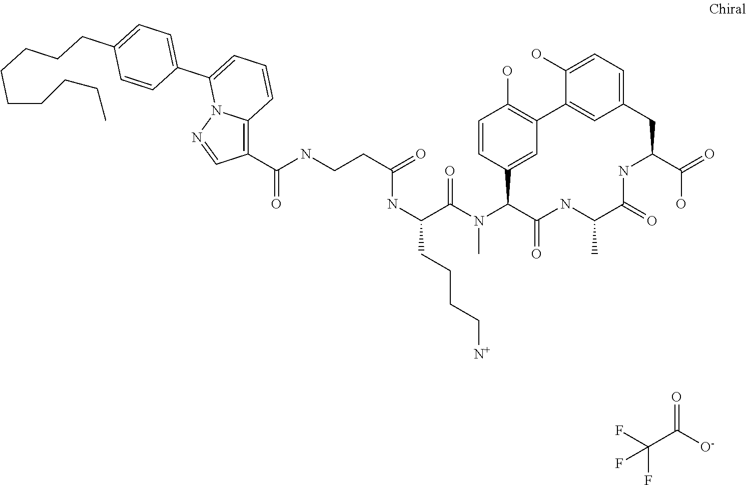 [SMILES: CCCCCCCCCc1ccc(-c2cccc3c(C(=O)NCCC(=O)N[C@@H](CCCC[NH3+])C(=O)N(C)[C@@H]4C(=O)N[C@@H](C)C(=O)N[C@H](C(=O)O)Cc5ccc(O)c(c5)-c5cc4ccc5O)cnn23)cc1 ; O=C([O-])C(F)(F)F] | 1073.189 |
| 66 | 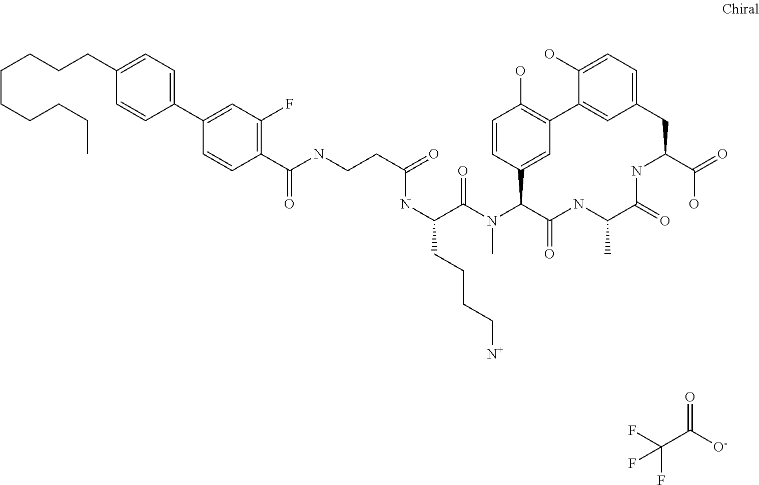 [SMILES: CCCCCCCCCc1ccc(-c2ccc(C(=O)NCCC(=O)N[C@@H](CCCC[NH3+])C(=O)N(C)[C@@H]3C(=O)N[C@@H](C)C(=O)N[C@H](C(=O)O)Cc4ccc(O)c(c4)-c4cc3ccc4O)c(F)c2)cc1 ; O=C([O-])C(F)(F)F] | 1051.155 |
| 67 | 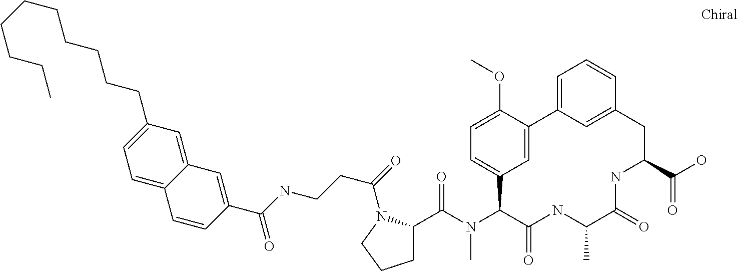 [SMILES: CCCCCCCCCCc1ccc2ccc(C(=O)NCCC(=O)N3CCC[C@H]3C(=O)N(C)[C@@H]3C(=O)N[C@@H](C)C(=O)N[C@H](C(=O)O)Cc4cccc(c4)-c4cc3ccc4OC)cc2c1] | 874.099 |

### Example 68

### Inhibition of SpsB Enzyme Activity

Signal peptidase enzyme activity was measured in a fluorescence-based assay using a bacterial membrane fraction as a source of SpsB (See Bruton et al., 2003, Eur J Med Chem 38:351-356). The SpsB substrate is a synthetic lipopeptide, decanoyl-K(DABCYL)-TPTAKA↓ASKKD-D(EDANS)—NH₂ (prepared by JPT Peptide Technologies, Berlin, Germany). Assays were performed with 20 μM peptide substrate (Km≈5 μM) and reactions were initiated with enzyme addition (final protein concentration of 0.6 mg/mL). SpsB mediated cleavage of the peptide substrate was detected as an increase in fluorescence (excitation 340 nm/emission 460 nm). Results are in Table 1.

### Determination of Minimum and Synergistic Inhibitory Concentration

MB 5393 (COL), a methicillin-resistant S. aureus strain, was inoculated into Trypticase Soy Broth (TSB, BBL) and grown in a humidified incubator for 18 hours with rotary shaking at 220 rpm and kept on ice until used.

A quantity of imipenem (Merck Chemical Collection) was weighed out and dissolved into sterile 10 mM MOPS buffer at pH 7. This solution was diluted to 1600 μg/mL, 200 μg/mL, and 25 μg/mL and filter-sterilized through a 0.45 μm filter. From these solutions, seven 1:2 serial dilutions were prepared using sterile 10 mM MOPS buffer at pH 7. The final concentration ranges tested were 128 to 2 μg/mL, 16 to 0.25 m/mL, and 2 to 0.03 μg/mL. The solutions were kept refrigerated until used.

Test compounds (EXAMPLES 1-67) were prepared in sterile water or DMSO at a concentration of 3.2 mg/mL. From this solution, 11 serial 1:2 dilutions were prepared using sterile water. The final concentration range tested was 64 to 0.0313 μg/mL. The solutions were kept refrigerated until used.

Two-fold serial dilutions of test compounds were tested in the presence and absence of subinhibitory concentrations of imipenem (4 μg/mL) in 96-well U bottom microliter dishes in microbiological growth media. The plates were inoculated with bacterial cells to a final concentration of ˜5×10⁵ CFU/mL. Test plates were incubated stationery at 37° C. for 22-24 hours.

After 22 hours of incubation, the Minimum inhibitory concentrations (MICs) were determined, defined as the lowest concentration of antibiotic that prevented all visible growth in the absence of imipenem. Synergistic inhibitory concentrations (SICs) were determined, defined as the lowest concentration of antibiotic that prevented all visible growth in the presence of imipenen. The results are in Table 1.

TABLE 1
Enzymatic and antimicrobial activities of the compounds
described in EXAMPLES 1 to 67

| | ¹SpsB | ²MIC (μg/mL) | ³SIC (μg/mL) |
|---|---|---|---|
| | A: IC₅₀ <10 nM | D: MIC <1 | G: SIC <0.0625 |
| | B: IC₅₀ 10-50 nM | E: MIC 1-8 | H: SIC 0.062-1 |
| | C: IC₅₀ >50 nM | F: MIC >8 | I: SIC >1 |
| EXAMPLE 1 | B | E | H |
| EXAMPLE 2 | B | E | H |
| EXAMPLE 3 | A | D | H |
| EXAMPLE 4 | C | F | I |
| EXAMPLE 5 | A | D | H |
| EXAMPLE 6 | B | D | H |
| EXAMPLE 7 | B | D | H |
| EXAMPLE 8 | B | E | H |
| EXAMPLE 9 | B | F | H |
| EXAMPLE 10 | A | E | H |
| EXAMPLE 11 | B | F | H |
| EXAMPLE 12 | B | F | H |
| EXAMPLE 13 | C | F | I |
| EXAMPLE 14 | A | D | G |
| EXAMPLE 15 | A | D | G |
| EXAMPLE 16 | A | D | G |
| EXAMPLE 17 | A | D | G |
| EXAMPLE 18 | A | E | H |
| EXAMPLE 19 | A | E | G |
| EXAMPLE 20 | A | E | G |
| EXAMPLE 21 | A | D | G |
| EXAMPLE 22 | A | D | G |
| EXAMPLE 23 | A | D | G |
| EXAMPLE 24 | A | D | G |
| EXAMPLE 25 | A | D | G |
| EXAMPLE 26 | A | D | G |
| EXAMPLE 27 | A | D | G |
| EXAMPLE 28 | A | D | G |
| EXAMPLE 29 | A | D | G |
| EXAMPLE 30 | A | D | G |
| EXAMPLE 31 | A | D | G |
| EXAMPLE 32 | A | D | G |
| EXAMPLE 33 | A | F | G |
| EXAMPLE 34 | A | D | G |
| EXAMPLE 35 | A | D | G |
| EXAMPLE 36 | A | D | G |
| EXAMPLE 37 | A | D | G |
| EXAMPLE 38 | A | D | G |
| EXAMPLE 39 | A | D | G |
| EXAMPLE 40 | A | D | G |
| EXAMPLE 41 | A | D | G |
| EXAMPLE 42 | A | D | H |
| EXAMPLE 43 | A | D | G |
| EXAMPLE 44 | A | D | G |
| EXAMPLE 45 | A | E | G |
| EXAMPLE 46 | A | D | G |
| EXAMPLE 47 | A | E | G |
| EXAMPLE 48 | A | E | H |
| EXAMPLE 49 | A | D | G |
| EXAMPLE 50 | A | F | H |
| EXAMPLE 51 | A | D | G |
| EXAMPLE 52 | A | E | G |
| EXAMPLE 53 | A | D | G |
| EXAMPLE 54 | A | D | G |
| EXAMPLE 55 | A | D | H |
| EXAMPLE 56 | A | E | H |
| EXAMPLE 57 | A | D | G |
| EXAMPLE 58 | A | D | G |
| EXAMPLE 59 | A | E | H |
| EXAMPLE 60 | B | E | G |
| EXAMPLE 61 | B | D | H |
| EXAMPLE 62 | B | D | G |
| EXAMPLE 63 | B | D | G |
| EXAMPLE 64 | B | D | G |
| EXAMPLE 65 | B | E | G |
| EXAMPLE 66 | B | D | G |
| EXAMPLE 67 | B | F | I |

Compounds provided in the Examples generally have IC₅₀ values for SpsB less than 30 nM and MIC values against the MRSA Col strain of less than 8 μg/ml. Moreover, many of the compounds provided in the Examples demonstrate a high degree of synergy with the carbapenem antibiotic, imipenem.

### Example 69

### Checkerboard Studies

The checkerboard method is the technique most frequently used to assess antimicrobial combinations in vitro. See Antibiotics in Laboratory Medicine, Victor Lorian ed., 2005). The checkerboard comprises a two-dimensional dilution scheme: columns in which each well contains the same amount of Drug A being two-fold diluted along the x-axis, and rows in which each well contains the same amount of Drug B being two-fold diluted on the Y-axis. The result is that each well contains a unique combination of the two drugs being tested. Also tested is the antimicrobial activity of each agent singly.

Imipenem was two-fold serially diluted along the Y-axis of 96-well U-bottom microtiter dishes (Fisher Scientific) in microbiological growth media (either brain heart infusion broth or cation-adjusted Mueller-Hinton broth +2% NaCl). The compounds represented in EXAMPLES 1-67 were serially diluted along the X-axis of 96-well U-bottom microtiter dishes in microbiological growth media. The plates were inoculated with bacterial cells (grown in Trypticase Soy Broth at 37° C. for 18 hours with rotary shaking) to a final concentration of 5×10⁵ CFU/mL. Test plates were incubated stationery at 37° C. for 22-24 hours. Minimum Inhibitory Concentrations (MICs) of each agent were defined as the minimum concentration of agent necessary to completely inhibit visible growth.

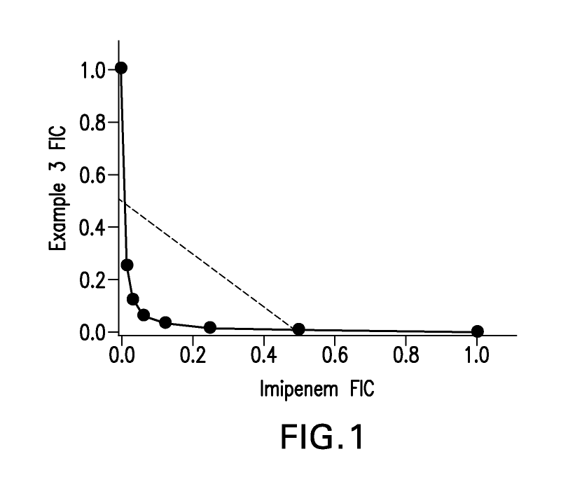

Synergy between SpsB inhibitors and β-lactam antibiotics is illustrated in the isobologram shown in FIG. 1. In this representation, data are expressed as fractional inhibitory concentrations (FIC), defined as the ratio between the MIC of one compound in the presence of the other, over its MIC alone. The FICs are plotted against each other and synergy is observed when at least one point falls below the synergy line which runs from 0.5 on one axis to 0.5 on the other. This line represents the points at which the sum of the FICs equals 0.5, below which significant deviation from simple additivity occurs, taking into account the 2-fold error inherent in the assay. The isobologram in FIG. 1 clearly demonstrate the synergistic effect of combining Imipenem and Example 3 in an in vitro experiment measuring bacterial growth. Addition of increasing amount of Example 3 to Imipenem or inversely addition of increasing amount of Imipenem to Example 3 lowers their respective MICs which is indicative of synergy.

### Example 70

### Combination Studies on Multidrug Resistant Staphylococcus aureus in Mouse Models

### Disseminated and Deep Thigh MRSA Infection Mouse Models

Female mice BALB/c mice (20-25 g) were rendered neutropenic via intraperitoneal injection of cyclophosphamide (Mead Johnson Pharmaceuticals) on Day −4 (250 mg/kg, disseminated) or Days −4/−1 (150/100 mg/kg, deep thigh) prior to experimental infection. Neutropenic mice were infected on Day 0 via intraperitoneal (disseminated) or intramuscular (deep thigh) injection of 0.1 ml containing ˜1-5×10⁴ CFU Staphylococcus aureus (strain B, MRSA COL). Linezolid (Zyvox IV solution, Bell Medical) was employed as an assay control. Fifteen minutes post infection, increasing doses of Example 3 (20, 40 and 80 mg/kg) or vehicle were administered subcutaneously (SC) TID with or without Imipenem/Cilastatin (6/50 mg/kg SC TID, disseminated; 10/50 mg/kg SC TID, deep thigh). Twenty four hours post dosing, mice were euthanized and infected kidneys (disseminated) or thighs (deep thigh) were aseptically removed, placed in 4 ml sterile phosphate buffered saline (Fisher Scientific), and homogenized using a Polytron (Brinkmann Instruments). Homogenates were serially 100-fold diluted in 9.9 ml sterile saline and plated on Mannitol Salt Agar plates. Plates were incubated at 35° C. for 48 hours and colony forming units (CFU) of bacteria remaining per thigh were determined.

### Results

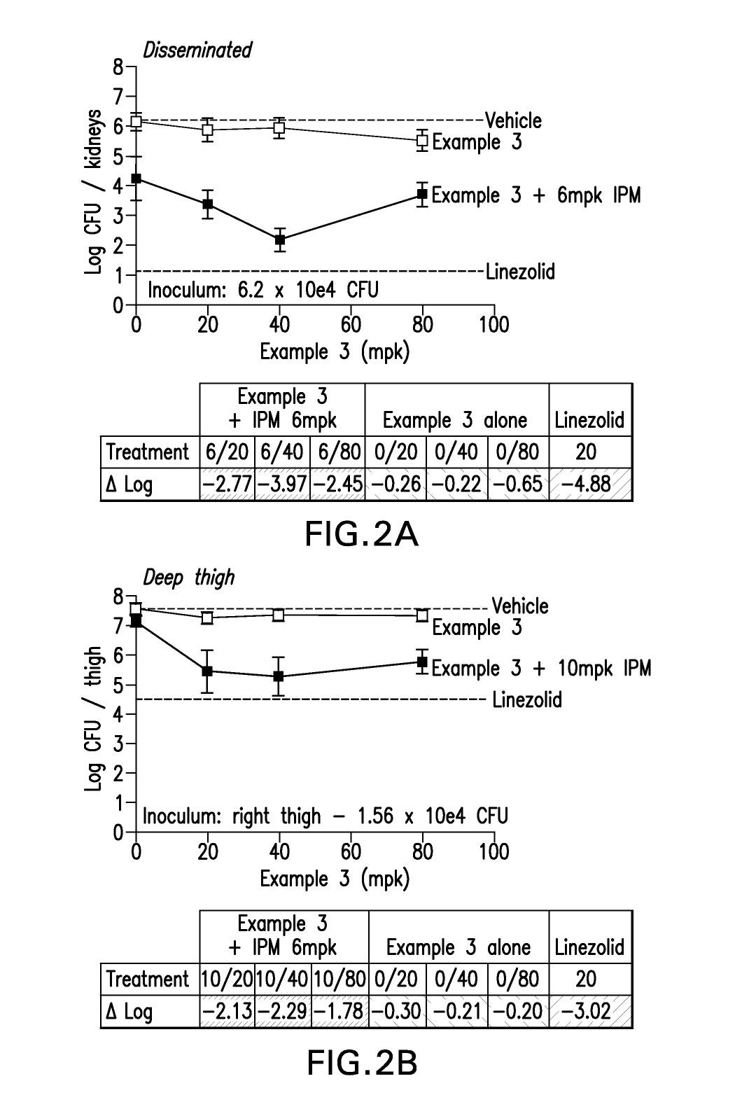

Relative to vehicle-treated, MRSA COL-infected animals in the disseminated (FIG. 2A) or deep thigh (FIG. 2B) model, the administration of increasing doses of Example 3 alone were shown to exert negligible anti-bacterial effects on MRSA COL bacterial burden. Co-administration of these doses of Example 3 with a non-efficacious dose of Imipenem/Cilastatin (6 or 10 mg/kg SC TID in the disseminated or deep thigh models, respectively) resulted in a 2-3 log reduction in bacterial burden, consistent with Example 3-dependent potentiation of Imipenem activity.

1. A compound of formula I: 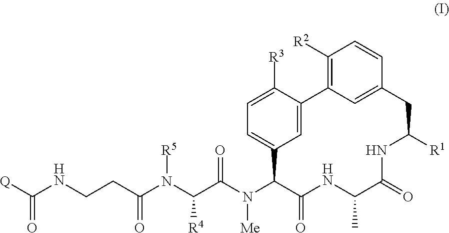
or a pharmaceutically acceptable salt thereof, wherein
R¹ is selected from C(R⁶)O, C(R⁶)₂OR⁶, COOR⁶ or CONR⁷R⁸;
R² and R³ are independently selected from H, halogen, OR⁶, SR⁶, SO₂R⁶, and NR⁷R⁸;
R⁴ and R⁵ are independently selected from hydrogen, C₁ to C₂₁ alkyl, cycloalkyl, alkenyl, cycloalkenyl and aryl, wherein the alkyl, cycloalkyl, alkenyl, cycloalkenyl or aryl is optionally substituted with one or more of C₁-C₄ alkyl, —NR⁷R⁸, guanidine, —OR⁶, OCONR⁷R⁸, COR⁶, CONR⁷R⁸, CN, SOR⁶, SO₂R⁶, SO₂NR⁷R⁸, F, Cl, Br, I or CF₃;
or R⁴ and R⁵ together with the atoms to which they are directly attached form a 4- to 5-membered heterocycle, optionally substituted with one or more of C₁-C₄ alkyl, —NR⁷R⁸, guanidine, —OR⁶, OCONR⁷R⁸, COR⁶, CONR⁷R⁸, CN, SOR⁶, SO₂R⁶, SO₂NR⁷R⁸, F, Cl, Br, I or CF₃;
Q is AryA or HetA;
AryA is an aryl optionally substituted with one or more of AryB, R⁶ optionally substituted with AryB, C₁ to C₂₁ alkyl optionally substituted with AryB, C₁ to C₂₁ alkenyl optionally substituted with AryB, or C₁ to C₂₁ alkynyl optionally substituted with AryB;
HetA is a heteroaryl optionally substituted with one or more of AryB, R⁶ optionally substituted with AryB, C₁ to C₂₁ alkyl optionally substituted with AryB, C₁ to C₂₁ alkenyl optionally substituted with AryB, or C₁ to C₂₁ alkynyl optionally substituted with AryB;
AryB is an aryl optionally substituted with a C₁ to C₂₁ alkyl or phenyl;
R⁶, R⁷, R⁸, are independently selected from H and C₁ to C₆ alkyl, wherein the alkyl is optionally substituted with one or more of —OR⁹, OCONR¹⁰R¹¹, OCOR⁹, COR⁹, CO₂R⁹, CONR¹⁰R¹¹, CN, SOR⁹, SO₂R⁹, SO₂NR¹⁰R¹¹, F, Cl, Br, I or CF₃ and
R⁹, R¹⁰, and R¹¹ are independently selected from H and C₁ to C₄ alkyl.

2. The compound of claim 1 wherein
R¹ is CH₂OH, COOH or CONH₂.

3. The compound of claim 1 wherein
R² and R³ are independently selected from H and OR⁶.

4. The compound of claim 3 wherein
R² and R³ are independently selected from H, OH and OCH₃.

5. The compound of claim 1 wherein
R⁴ and R⁵ are H or C₁ to C₂₁ alkyl, wherein the alkyl is optionally substituted with amine, guanidine or —NR⁷R⁸.

6. The compound of claim 1 wherein R⁴ and R⁵ are H or C₁ to C₂₁ alkyl, wherein the alkyl is optionally substituted with amine, guanidine or —NR⁷R⁸ and wherein R⁷ and R⁸ are independently selected from C₁ to C₆ alkyl.

7. The compound of claim 1 wherein
Q is

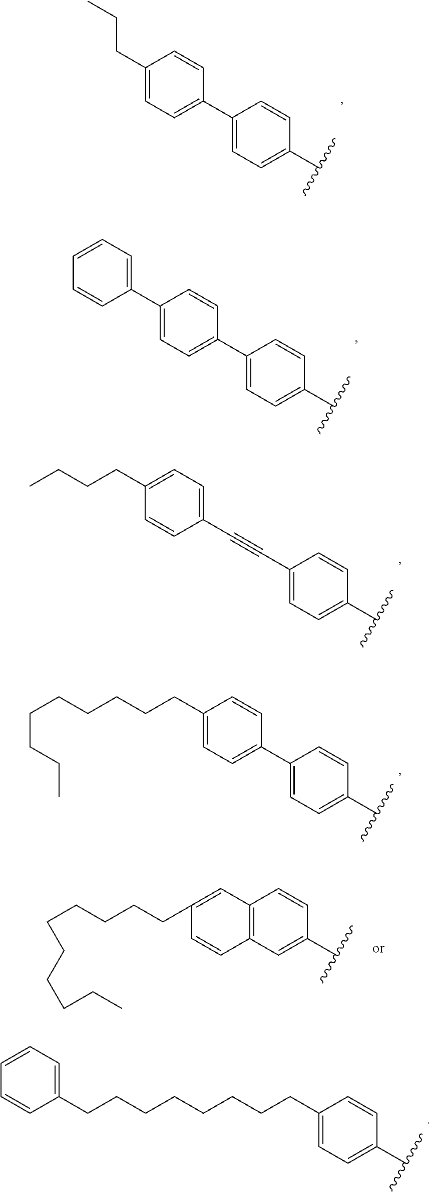 [SMILES: *c1ccc(-c2ccc(CCC)cc2)cc1 ; *c1ccc(-c2ccc(-c3ccccc3)cc2)cc1 ; *c1ccc(C#Cc2ccc(CCCC)cc2)cc1 ; *c1ccc(-c2ccc(CCCCCCCCC)cc2)cc1 ; *c1ccc2cc(CCCCCCCCCC)ccc2c1 ; *c1ccc(CCCCCCCCc2ccccc2)cc1]

8. The compound of claim 1, wherein Q is 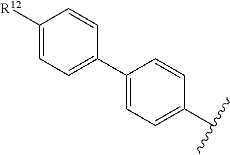
wherein R¹² is a C₁ to C₁₂ alkyl.

9. The compound of claim 1 wherein the compound is selected from the group consisting of:
6-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
5-(R,S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
(5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
(5R)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
(5R,S)-6-[[(7S,10S,13S)-3,18-dihydroxy-13-(hydroxymethyl)-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
6-[[(7S,10S,13S)-13-(aminocarboxyl)-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-({3-[(1,1′:4′,1″-terphenyl-4-ylcarbonyl)amino]propanoyl}amino)hexan-1-aminium;
({(4S)-5-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-oxo-4-[(3-{[(4′-propylbiphenyl-4-yl)carbonyl]amino}propanoyl)amino]pentyl}amino) (imino)methanaminium;
6-[[(7S,10S,13S)-13-carboxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
5-{[3-({4-[(4-butylphenyl)ethynyl]benzoyl}amino)propanoyl]amino}-6-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxohexan-1-aminium;
4-[{2-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-2-oxoethyl}(3-{[(4′-propylbiphenyl-4-yl)carbonyl]amino}propanoyl)amino]butan-1-aminium;
(8S,11S,14S)-3,18-dihydroxy-11-methyl-14-(methyl{[(2S)-1-(3-{[(4′-propylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(8S,11S,14S)-3,18-dimethoxy-11-methyl-14-(methyl{[(2R)-1-(3-{[(4′-propylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(8S,11S,14S)-3,18-dihydroxy-11-methyl-14-(methyl{[(2R)-1-{3-[(6-decyl-2-naphthoyl)amino]propanoyl}pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(5S)-6-[[(7S,10S,13S)-13-carboxy-3,18-dihydroxy-10-methyl-8,11-dioxo-9,12-diazatricyclo[13.3.1.12,6]icosa-1(19),2(20),3,5,15,17-hexaen-7-yl](methyl)amino]-6-oxo-5-[(3-{[4-(8-phenyloctyl)benzoyl]amino}propanoyl)amino]hexan-1-aminium;
(5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-[(3-{[(4′-nonylbiphenyl-4-yl)carbonyl]amino}propanoyl)amino]-6-oxohexan-1-aminium;
(5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-({3-[(6-decyl-2-naphthoyl)amino]propanoyl}amino)-6-oxohexan-1-aminium;
(8S,11S,14S)-3,18-dihydroxy-11-methyl-14-(methyl{[(2)-1-(3-{[(4′-nonylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(8S,11S,14S)-3,18-dihydroxy-11-methyl-14-(methyl{[(2)-1-(3-{[(4′-nonylbiphenyl-4-yl)carbonyl]amino}propanoyl)azetidin-2-yl]carbonyl}amino)-10,13-dioxo-9,12-diazatricyclo[13.3.1.1^{2,6}]icosa-1(19),2(20),3,5,15,17-hexaene-8-carboxylic acid; and
(8S,11S,14S)-3,18-dihydroxy-11-methyl-14-(methyl{[(2R)-1-{3-[(6-decyl-2-naphthoyl)amino]propanoyl}azetidin-2-yl]carbonyl}amino)-10,13-dioxo-9,12-diazatricyclo[13.3.1.1^{2,6}]icosa-1(19),2(20),3,5,15,17-hexaene-8-carboxylic acid.

10. A pharmaceutical composition comprising an effective amount of a compound of claim 1 and a pharmaceutically acceptable carrier.

11. The pharmaceutical composition of claim 10 further comprising an effective amount of a β-lactam antibiotic.

12. The pharmaceutical composition of claim 11, wherein the β-lactam antibiotic is a carbapenem, cephalosporin, monolactam or penicillin.

13. The pharmaceutical composition of claim 12, wherein the β-lactam antibiotic is a carbapenem antibiotic.

14. The pharmaceutical composition of claim 13, wherein the carbapenem antibiotic is imipenem or ertapenem.

15. (canceled)

16. (canceled)

17. (canceled)

18. A method of treating a bacterial infection in a mammalian patient in need of such treatment comprising administering to said patient a compound of claim 1 in combination with a pharmaceutically acceptable β-lactam antibiotic in an amount which is effective for treating a bacterial infection.

19. The method of claim 18 wherein said bacterial infection is methicillin-resistant S. aureus or methicillin-resistant S. epidermis.

20. The method of claim 18, wherein the β-lactam antibiotic is a carbapenem, cephalosporin, monolactam or penicillin.

21. The method of claim 20, wherein the β-lactam antibiotic is a carbapenem antibiotic.

22. The method of claim 21, wherein the carbapenem antibiotic is imipenem or ertapenem.

23. (canceled)

24. (canceled)

25. (canceled)

26. The method of claim 18, wherein the compound of claim 1 and β-lactam antibiotic are administered sequentially.

27. The method of claim 26, wherein the compound of claim 1 and β-lactam antibiotic are administered within one hour of each other.

28. The method of claim 18 wherein the compound of claim 1 and β-lactam antibiotic are administered concurrently.

29. The method of claim 28 wherein the compound of claim 1 and β-lactam antibiotic are administered in the same formulation.

30. The method of claim 18 wherein the compound is selected from the group consisting of:
6-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
5-(R,S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
(5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
(5R)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
(5R,S)-6-[[(7S,10S,13S)-3,18-dihydroxy-13-(hydroxymethyl)-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
6-[[(7S,10S,13S)-13-(aminocarboxyl)-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-({3-[(1,1′:4′,1″-terphenyl-4-ylcarbonyl)amino]propanoyl}amino)hexan-1-aminium;
({(4S)-5-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-oxo-4-[(3-{[(4′-propylbiphenyl-4-yl)carbonyl]amino}propanoyl)amino]pentyl}amino) (imino)methanaminium;
6-[[(7S,10S,13S)-13-carboxy-10-methyl-8,11-dioxo-9,12-diazatricyclo[13.3.1.1^{2,6}]icosa-1(19),2(20),3,5,15,17-hexaen-7-yl](methyl)amino]-6-oxo-5-[(3-{[(4′-propylbiphenyl-4-yl)carbonyl]amino}propanoyl)amino]hexan-1-aminium;
5-{[3-({4-[(4-butylphenyl)ethynyl]benzoyl}amino)propanoyl]amino}-6-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-6-oxohexan-1-aminium;
4-[{2-[[(7S,10S,13S)-13-carboxy-3,18-dimethoxy-10-methyl-8,11-dioxo-9,12-diazatricyclo[13.3.1.1^{2,6}]icosa-1(19),2(20),3,5,15,17-hexaen-7-yl](methyl)amino]-2-oxoethyl}(3-{[(4′-propylbiphenyl-4-yl)carbonyl]amino}propanoyl)amino]butan-1-aminium;
(8S,11S,14S)-3,18-dihydroxy-11-methyl-14-(methyl{[(2S)-1-(3-{[(4′-propylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(8S,11S,14S)-3,18-dimethoxy-11-methyl-14-(methyl{[(2R)-1-(3-{[(4′-propylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(8S,11S,14S)-3,18-dihydroxy-11-methyl-14-(methyl{[(2R)-1-{3-[(6-decyl-2-naphthoyl)amino]propanoyl}pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(5S)-6-[[(7S,10S,13S)-13-carboxy-3,18-dihydroxy-10-methyl-8,11-dioxo-9,12-diazatricyclo[13.3.1.12,6]icosa-1(19),2(20),3,5,15,17-hexaen-7-yl](methyl)amino]-6-oxo-5-[(3-{[4-(8-phenyloctyl)benzoyl]amino}propanoyl)amino]hexan-1-aminium;
(5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-[(3-{[(4′-nonylbiphenyl-4-yl)carbonyl]amino}propanoyl)amino]-6-oxohexan-1-aminium;
(5S)-6-[[(7S,10S,13S)-13-carboxy-3,18-dihydroxy-10-methyl-8,11-dioxo-9,12-diazatricyclo[13.3.1.1^{2,6}]icosa-1(19),2(20),3,5,15,17-hexaen-7-yl](methyl)amino]-5-({3-[(6-decyl-2-naphthoyl)amino]propanoyl}amino)-6-oxohexan-1-aminium;
(8S,11S,14S)-3,18-dihydroxy-11-methyl-14-(methyl{[(2)-1-(3-{[(4′-nonylbiphenyl-4-yl)carbonyl]amino}propanoyl)pyrrolidin-2-yl]carbonyl}amino)-10,13-dioxo-9,12-diazatricyclo[13.3.1.1^{2,6}]icosa-1(19),2(20),3,5,15,17-hexaene-8-carboxylic acid;
(8S,11S,14S)-3,18-dihydroxy-11-methyl-14-(methyl{[(2)-1-(3-{[(4′-nonylbiphenyl-4-yl)carbonyl]amino}propanoyl)azetidin-2-yl]carbonyl}amino)-10,13-dioxo-9,12-diazatricyclo[13.3.1.1^{2,6}]icosa-1(19),2(20),3,5,15,17-hexaene-8-carboxylic acid; and
(8S,11S,14S)-3,18-dihydroxy-11-methyl-14-(methyl{[(2R)-1-{3-[(6-decyl-2-naphthoyl)amino]propanoyl}azetidin-2-yl]carbonyl}amino)-10,13-dioxo-9,12-diazatricyclo[13.3.1.1^{2,6}]icosa-1(19),2(20),3,5,15,17-hexaene-8-carboxylic acid.

31. (canceled)
